(19) United States
(12) Patent Application Publication
(10) Pub. No.: US 2024/0159775 A1
(43) Pub. Date: May 16, 2024

(54) WASTEWATER-BASED EPIDEMIOLOGY METHODS FOR ASSESSING POPULATION HEALTH

(71) Applicants: Rolf HALDEN, Phoenix, AZ (US); Sangeet ADHIKARI, Emeryville, CA (US); Erin DRIVER, Tempe, AZ (US); Rahul KUMAR, Patna (IN); Jacob ZEVITZ, Tempe, AZ (US); Devin A. BOWES, Tempe, AZ (US); Vivek AMIN, Phoenix, AZ (US)

(72) Inventors: Rolf HALDEN, Phoenix, AZ (US); Sangeet ADHIKARI, Emeryville, CA (US); Erin DRIVER, Tempe, AZ (US); Rahul KUMAR, Patna (IN); Jacob ZEVITZ, Tempe, AZ (US); Devin A. BOWES, Tempe, AZ (US); Vivek AMIN, Phoenix, AZ (US)

(73) Assignee: Arizona Board of Regents on Behalf of Arizona State University, Scottsdale, AZ (US)

(21) Appl. No.: 18/496,832

(22) Filed: Oct. 27, 2023

Related U.S. Application Data
(60) Provisional application No. 63/381,279, filed on Oct. 27, 2022.
(60) Provisional application No. 63/382,480, filed on Nov. 4, 2022.
(60) Provisional application No. 63/383,202, filed on Nov. 10, 2022.
(60) Provisional application No. 63/480,090, filed on Jan. 16, 2023.

(51) Int. Cl. G01N 33/68; G01N 33/53; G01N 33/573; G01N 33/574

(52) U.S. Cl. CPC G01N 33/6893; G01N 33/5308; G01N 33/573; G01N 33/57488; G01N 33/6848; G01N 2333/471; G01N 2333/8139; G01N 2333/912; G01N 2800/32

(57) ABSTRACT

Disclosed herein are methods of assessing a population's health using wastewater samples. For example, the methods assess exposure to pathogens, plasticizers, volatile organic compounds, surfactant, hazardous chemical, nicotine, and/or other environmental toxins. In some aspects, the methods determine the concentration of disease biomarkers, chemical biomarker, certain human hormones, certain genes, pharmaceuticals, illicit drugs, personal care product, pathogens, and/or endogenous mental health markers. In some aspects, the methods assess population health at a neighborhood level. The methods may also be used to assess population health at a larger regional level, for example at a state level or a national level.

### CROSS REFERENCE TO RELATED APPLICATIONS

This application claims the benefit of U.S. Provisional Application No. 63/382,480, entitled “METHODS OF ASSESSING POPULATION-WIDE EXPOSURE TO PLASTICIZERS AND PLASTIC MONOMERS,” which was filed Nov. 4, 2022, U.S. Provisional Application No. 63/381,279, entitled “URINARY PROTEIN BIOMARKERS RELATED TO CARDIOVASCULAR DISEASE AND CANCER AND USES THEREOF,” which was filed Oct. 27, 2022, U.S. Provisional Application No. 63/383,202, entitled “WASTEWATER-BASED EPIDEMIOLOGY METHODS OF TRACKING HEALTH AND PROSPERITY IN A POPULATION,” which was filed Nov. 10, 2022, and U.S. Provisional Application No. 63/480,090, entitled “WASTEWATER-BASED EPIDEMIOLOGY METHODS OF MEASURING EXPOSURE TO VOLATILE ORGANIC COMPOUNDS,” which was filed Jan. 16, 2023, the entire disclosure of each of which are hereby incorporated by reference.

### FIELD OF THE INVENTION

The invention relates to wastewater-based methods of assessing a population's health, including but not limited to assessing human exposure to plasticizers and plastic monomers, incidences of cardiovascular disease and case, and metrics for measuring social, economic, and environmental development milestones.

### BACKGROUND OF THE INVENTION

Since the industrial revolution, human civilization's impact on Earth has become increasingly more lasting. Part of this impact has results in subsequent effects of human health in view of increased exposures to toxic chemicals and metal (by amount or time). The best way to study and understand the effect of a human population's effect on its surrounding environment and the resulting effect of the environmental change on a human is through conducting population-wide studies. However, population studies are greatly limited when the analysis requires direct measurements. The difficulty is increased when these assessed of influence and relationship are often described qualitatively.

Since its invention in 1907, plastics have had several applications and have provided numerous societal benefits. However, these materials have negatively impacted human health and the environment due to the leaching of harmful endogenous chemical additives and monomers over time. Plasticizers are additives introduced into plastics to improve flexibility and processibility. Phthalate diesters have been used as plasticizers for decades. They are used as additives in numerous consumer products, including pharmaceuticals and personal care products (PPCPs), adhesives, food packaging, medical devices, and furnishings. Phthalates are categorized into low molecular phthalates (LMWPs) or high molecular weight phthalates (HMWPs) based on their alkyl chain length. Dimethyl phthalates and diethyl phthalates are examples of LMWPs, whereas di(2-ethylhexyl) phthalates and di-n-octyl phthalates are examples of HMWPs. LMWPs are more commonly found in cosmetics and other personal care products and are detected more often in human urine and environmental matrices. Importantly, most phthalates are potential endocrine disruptors that can interfere with human endogenous hormones, affecting normal growth and development and human well-being. Human exposure to phthalates mainly occurs through dietary sources, inhalation, and dermal contact. Phthalates have been ubiquitously measured in all environmental matrices, including air, water, soil, sludge, and dust. Phthalate diesters are quickly metabolized, owing to their short biological half-lives, to phthalate monoesters or other oxidative metabolites when humans are exposed to them. Phthalate urinary metabolites have been reported as an excellent biomarker of phthalate exposure.

Bisphenol A (BPA) is one of the oldest known endocrine disruptors and is used as a monomer to make polycarbonate (PC) plastics with added benefits of being lightweight, transparent, durable, and resistant to heat and chemicals. In addition, BPA is also used in the production of epoxy resins. BPA is now regulated in several countries due to its significant health impact. Several episodes of BPA toxicities have prompted industries to substitute BPA with other alternatives structurally similar to BPA, including BPF (Bisphenol F), BPS (Bisphenol S), and BPAF (Bisphenol AF). These chemicals with two hydroxyphenyl functional groups are termed bisphenol analogs. Bisphenol F and Bisphenol S are frequently detected bisphenol analogs in the environmental compartments.

Polyethylene terephthalate (PET) and PC are widely used plastics worldwide. Terephthalic acid is the monomer of PET and has been reported to leach from PET bottles. A recent study has reported frequent detection of PET and PC in sewage sludge collected from several wastewater treatment plants across the US. Occupational exposure to terephthalic acid has been reported to have no impact on normal human organ functionality in a study. However, more studies are needed to evaluate the toxicity of terephthalic acid at longer contact times.

Human exposure to plasticizers and plastic monomers is commonly assessed by measuring their concentrations in body fluids, including blood, urine, semen, and milk. However, the exposure assessment by individual specimen collection is invasive, time-consuming, and expensive. Accordingly, these methods cannot be used to evaluate community exposure to plasticizers and plastic monomers, especially at a near real-time scale with a significantly lower cost. Thus, there is a need for developing a method to study a community's exposure to plasticizers and plastic monomers that does not involve collecting specimen from individuals.

Globally, population-wide exposure to air pollution is a profound threat to public health and economic progress. Exposure to air pollution is one of the most significant environmental threats to public health and has been linked to millions of premature deaths annually worldwide. Almost 99% of the global population is exposed to levels of air pollutants that exceed World Health Organization guidelines. Indoor and outdoor exposure to air pollutants causes approximately seven million premature deaths every year. Poorer communities and communities of color are disproportionally impacted over by air pollution compared to other groups in the US, with children, the elderly, and those with preexisting conditions being the most vulnerable. Exposures to air pollution are associated with an increased risk of asthma, respiratory infections, cardiovascular disease, various forms of cancer, and death.

Volatile organic compounds (VOCs) a class of air pollutants consisting of more than a thousand chemicals generated in both indoor and outdoor environments, including vehicle emissions, various industrial processes, cooking, wood burning, building materials, cleaning supplies, and other household products. In the US, the Environmental Protection Agency (EPA) regulates outdoor air emissions of VOCs, targeting those compounds that photochemically react to produce tropospheric ozone, a potent greenhouse gas. Within indoor air, VOC exposures are indirectly mitigated through the regulation of various consumer household products. Unfortunately, this list is not inclusive, only limited to photochemically reactive compounds.

Although air pollution is monitored by measuring ambient particulate matter loadings, it is challenging to estimate indoor and outdoor exposure of individuals to air pollutants. Human exposure to VOCs can be monitored through a variety of mechanisms, including traditional routes of assessment, the collection of an individual's blood, urine, breath, or sweat, and less invasive, newer techniques, including low-cost air quality sensors (LCAQS) carried by the study participant, or the use of wearable silicone bracelets that act as passive samplers. In all cases, the studies require the active participation of individuals, the reliance on those individuals to provide a sample (invasive), to carry or wear the sampling device, and the assumption that the chemicals moving into the sampler are at a similar rate of exposure to those being inhaled by the individual. Additionally, there is an associated capital cost of individual LCAQS (US$2000-US$5000), and many are non-specific to the type of exposure (total volatile organic compound, TVOC). Although the cost of silicon bracelets is low, the subsequent VOC analysis is relatively high (US$1000/bracelet). Thus, novel methods need to be developed for estimating an individual's exposure to air pollutants, such as VOCs, that do not require collecting individual-specific information.

For several decades, cardiovascular disease (CVD) has remained as the leading cause of death worldwide, with cancer consistently ranked within the top ten. CVD and cancer are collectively responsible for tens of millions of global deaths each year. This is projected to intensify as the COVID-19 pandemic has caused delays in individualized diagnostics or exacerbated prevalence due to post-infection complications.

Both CVD and cancer are classified as noncommunicable chronic diseases as their etiology cannot be readily linked to an acute infectious agent but are rather influenced by a multitude of genetic, environmental, and/or lifestyle factors and behaviors, with clinical symptoms lasting for extended periods of time. If left untreated, the presence of a persistent chronic disease may increase the risk for co-morbidities or other severe complications to arise. While both cancer and CVD have a wide range of sub-classifications, their associated risk factors often overlap, such as diet, access to healthcare, or environmental exposures, which has been shown to disproportionately impact certain populations. In the United States, these diseases have made a tremendous impact on human life, with historical yearly death estimates reported at approximately 647,000 for CVD and nearly 600,000 for cancer. More recent reports from 2020 indicate these death rates are increasing, likely in part due to the COVID-19 global pandemic causing significant delays in individualized preventative or diagnostic measures, as well as a rise in post-infection complications. Thus, there is a need for additional and alternative strategies for obtaining rapid and comprehensive information on these complex fatal diseases.

The journey to establishing a diagnosis for CVD and cancer typically require intense and invasive procedures at the individual-level; oftentimes performed by multiple specialists and can include several rounds of biological specimen analyses (i.e., blood, urine, stool), comprehensive imaging, and physical exertion tests. These diagnostic procedures cannot only become time-consuming and expensive, with annual healthcare costs estimated at approximately $500 billion per year (USD) for these two diseases combined, but it can also cause a great deal of physical, mental, and emotional strain on the patient and the patient's support system. Multiple nationwide surveillance programs exist in the U.S. in order to understand population-level trends in risk factor behaviors, exposures, disease incidence, co-morbidities, and mortalities, amongst others, however, these broad-scale systems rely heavily on self-reported survey data that may be susceptible to recall bias. While these various data collection methods are informative, they may either limit insight to only the individuals who have access to healthcare and are afforded the flexibility to undergo multiple diagnostic procedures, or contextual and important information is lost due to low-resolution and time-delayed data collection from widescale national surveys. Ultimately, these limitations to conventional methods for chronic disease data acquisition may necessitate supplementary measurements in order to provide a comprehensive population-level health assessment that encompasses near real-time, contextually-relevant, non-invasive, and inclusive data.

With the rapid urbanization and evidence of climate change, the member countries of the United Nations (UN) have unanimously agreed on agendas for attaining sustainable social, economic, and environmental development milestones. With a target date of 2030, these agendas comprise 17 different sustainable development goals (SDGs). A common criticism, however, of the UN agenda is the difficulty in tracking these goals, which are framed as qualitative parameters. While quantifiable features can be assigned that could be used for tracking progress towards a particular SDG, the goals necessitate an evaluation within a population. As such, methods that provide population-level assessments without collecting data from individuals are needed.

### SUMMARY OF THE INVENTION

Disclosed herein are methods of assessing a population's health through studying wastewater sample from the population. The methods comprise determining the concentration of at least one analyte in the wastewater sample, wherein the at least one analyte is selected from the group consisting of: a hunger hormone, a stress hormone, cardiovascular disease biomarker, pulmonary disease biomarker, cancer biomarker, illicit drug, personal care product, surfactant, hazardous chemical, disease resistant pathogen, an antimicrobial resistance gene, a psychotropic drug, an alcohol-related chemical biomarker, a metabolite of volatile organic compounds (VOCs), an environmental toxin, a nicotine-related metabolite, a plastic component, and endogenous mental health marker. The concentration of the aforementioned analytes can assess the population's exposure to plasticizers and plastic monomers, assess the population's exposure to VOCs, estimate the prevalence of cardiovascular disease (CVD) and cancer within the population, and track health and prosperity parameters in a population, such as the UN's SDGs. In some implementations, the concentration of the at least one analyte is determined using at least one method selected from the group consisting of: liquid chromatography (LC), gas chromatography (LC), mass spectrometry (MS), flame ionization detection (FID), and electron capture detection (ECD). In a particular embodiment, the concentration of the at least one analyte is determined using LC and MS or determined using GC and MS.

In some aspects, the methods further comprise normalizing the concentration of the at least one analyte in the wastewater sample with at least one normalization agent. In some implementations, the wastewater sample is frozen upon collection and thawed before determining the concentration of the at least one analyte. In particular implementations, the concentration of the at least one analyte in the wastewater sample is determined within two weeks of collecting the wastewater sample. Thus, in some embodiments, the thawed wastewater sample is provided for determining the analyte concentration within two weeks of collecting and freezing the wastewater sample.

For tracking changes in the population, the method of assessing a population's health comprises providing a first wastewater sample from the population and providing a second wastewater sample from the population, wherein the second wastewater sample is collected from a same location as the first wastewater sample and is collected at least two days after collecting the wastewater fluid sample. The method further comprises detecting the concentration of at least one analyte in the first wastewater sample and in the second wastewater sample and identifying a change in the population's health upon detection that the concentration of the at least one biomarker is changed between the first wastewater sample and the second wastewater sample.

In one aspect, disclosed herein are methods of assessing human exposure to plasticizers and plastic monomers at a population level, for example at a neighborhood level, at a state level, or at a national level. The methods involve collecting wastewater at a neighborhood level and determining the concentration of plasticizer metabolites in urine. In some aspects, the methods assess exposure to phthalates, bisphenols (BPs), and terephthalic acid (TPA) by measuring urinary metabolites in community wastewater. The analytes assessed are selected from bisphenol A (BPA), BPA monosulfate, Bisphenol S, monobenzyl phthalate (MBzP), mono (2-ethyl-5-hydroxyhexyl) phthalate (MEOHP), mono-2-ethylhexyl phthalate (MEHP), monoethyl phthalate (MEP), monobutyl phthalate (MMP), mono-n-octyl phthalate (MnOP), TPA, monobutyl phthalate (MBP), monoisobutyl phthalate (MiBP), mono-(2-ethyl-5-oxohexyl) phthalate (MEOHP), or any combination thereof. In some implementations, the methods comprise determining the concentration of at least MEHP. In certain implementations, the collected wastewater sample is frozen upon collection and then thawed at sample processing. In some aspects, the concentration of the analyte is determined within two weeks or within one week of sample collection.

In another aspects, disclosed herein are urinary protein biomarkers for diagnosing cardiovascular disease and cancer. Accordingly, also disclosed are methods of diagnosing or monitoring cardiovascular disease and cancer in a subject as well as methods of monitoring or assessing the prevalence of cardiovascular disease, cancer, or both in a population, ranging in scale from a single dwelling, a hospital, an entire city, a nation, a continent, and the world.

In some aspects, a method of assessing the prevalence of cardiovascular disease (CVD) and/or cancer in a population is described. In one embodiment, the method comprises providing a fluid sample comprising urine from the population; detecting the concentration of at least one biomarker selected from cystatin C and microtubule assisted serine/threonine kinase 4 (MAST4) in the fluid sample; and identifying the population as containing a significant portion of subjects with CVD and/or cancer upon detection of at least one biomarker at a concentration of at least 0.1 ng/L/10,000 population. In some implementations, the method further comprises detecting the concentration of cardiac troponin I (cTnI), α-fetoprotein, and/or normetanephrine; and identifying the population as containing a significant portion of subjects with CVD and/or cancer upon detection of cTnI, α-fetoprotein, or normetanephrine at a concentration of at least 0.1 ng/L/10,000 population.

In another embodiment, the method comprises providing a fluid sample comprising urine from the population; detecting the concentration of at least one biomarker selected from the group consisting of: cTnI, cystatin C, α-fetoprotein, normetanephrine, and microtubule assisted serine/threonine kinase 4 (MAST4), in the fluid sample; normalizing the concentration of the at least one biomarker with a normalization agent (for example, creatinine and coprostanol); and identifying the population as containing a significant portion of subjects with CVD and/or cancer upon detection of at least one biomarker at a concentration of at least 0.1 ng/L/10,000 population.

In yet another embodiment, the method of assessing the prevalence of CVD and/or cancer in a population comprises providing a first fluid sample from the population; detecting the concentration of at least one biomarker selected from cystatin C and MAST4 in the first fluid sample; providing a second fluid sample from the population, wherein the second fluid sample is collected from a same location as the first fluid sample and is collected at least two days after collecting the first fluid sample; detecting the concentration of at least one biomarker in the second fluid sample, and identifying the population as having increase incidence of CVD and/or cancer upon detection that the concentration of the at least one biomarker is higher in the second fluid sample than the first fluid sample. In some implementations, the method further comprises the concentration of at least one other biomarker selected from cTnI, α-fetoprotein, and/or normetanephrine in the first fluid sample; and detecting the concentration of the at least one other biomarker in the second fluid sample. The population is identified as having increase incidence of CVD and/or cancer upon detection that the concentration of the at least one biomarker and the concentration of the at least one other biomarker is higher in the second fluid sample than the first fluid sample.

In still another embodiment, the method of assessing the prevalence of CVD and/or cancer in a population comprises providing a first fluid sample from the population; detecting the concentration of at least one biomarker selected from the group consisting of: cTnI, cystatin C, α-fetoprotein, normetanephrine, and microtubule assisted serine/threonine kinase 4 (MAST4), in the fluid sample; normalizing the concentration of the at least one biomarker in the first fluid sample with a normalization agent (for example, creatinine and coprostanol); providing a second fluid sample from the population, wherein the second fluid sample is collected from a same location as the first fluid sample and is collected at least two days after collecting the first fluid sample; detecting the concentration of at least one biomarker in the second fluid sample; normalizing the concentration of the at least one biomarker in the second fluid sample with the normalization agent; and identifying the population as having increase incidence of CVD and/or cancer upon detection that the concentration of the at least one biomarker is higher in the second fluid sample than the first fluid sample.

Also described is a method of assessing the prevalence of CVD in a population. The method comprises providing a fluid sample comprising urine from the population; detecting the concentration of cystatin C in the fluid sample; and identifying the population as containing a significant portion of subjects with CVD upon detection of at least one biomarker at a concentration of at least 1.2×10³ ng/L/10,000 population. In some implementations, the method further comprises detecting the concentration of cTnI; and identifying the population as containing a significant portion of subjects with CVD upon detection of cTnI at a concentration of at least 0.2 ng/L/10,000 population. In some aspects, the method also comprises normalizing the concentration of cystatin C and/or cTnI in the fluid sample with a normalization agent (for example, creatinine and coprostanol).

In another embodiment, the method of assessing the prevalence of CVD in a population comprises providing a first fluid sample from the population; detecting the concentration of cystatin C, in the first fluid sample; providing a second fluid sample from the population, wherein the second fluid sample is collected from a same location as the first fluid sample and is collected at least two days after collecting the first fluid sample; detecting the concentration of cystatin C in the second fluid sample, and identifying the population as having an increase incidence of CVD upon detection that the concentration of cystatin C is higher in the second fluid sample than the first fluid sample. In some implementations, the method further comprises detecting the concentration of cTnI in the first fluid sample; and detecting the concentration of cTnI in the second fluid sample. The population is identified as having an increase incidence of CVD and/or cancer upon detection that the concentration of cystatin C and cTnI is higher in the second fluid sample than the first fluid sample. In some aspects, the method also comprises normalizing the concentration of cystatin C and/or cTnI in the fluid samples with a normalization agent (for example, creatinine and coprostanol).

A method of assessing the prevalence of cancer (for example, breast cancer) in a population is further described. The method comprises providing a fluid sample comprising urine from the population; detecting the concentration of MAST4 in the fluid sample; and identifying the population as containing a significant portion of subjects with cancer upon detection of at least one biomarker at a concentration of at least 0.1 ng/L/10,000 population. In some implementations, the method further comprises detecting the concentration of normetanephrine and/or α-fetoprotein; and identifying the population as containing a significant portion of subjects with cancer upon detection of normetanephrine at a concentration of at least 6.89×10² ng/L/1,000 population and/or α-fetoprotein at a concentration of at least 0.1 ng/L/10,000 population. In some aspects, the method also comprises normalizing the concentration of MAST4, normetanephrine, and/or α-fetoprotein in the fluid sample with a normalization agent (for example, creatinine and coprostanol).

In another embodiment, the method of assessing the prevalence of cancer in a population comprises providing a first fluid sample from the population; detecting the concentration of MAST4 in the first fluid sample; providing a second fluid sample from the population, wherein the second fluid sample is collected from a same location as the first fluid sample and is collected at least two days after collecting the first fluid sample; detecting the concentration MAST4 in the second fluid sample, and identifying the population as having an increase incidence of cancer upon detection that the concentration of MAST4 is higher in the second fluid sample than the first fluid sample. The method may further comprise detecting the concentration of at least one other biomarker selected from α-fetoprotein and normetanephrine in the first fluid sample; and detecting the concentration of the at least one other biomarker in the second fluid sample. The population is identified as having an increase incidence of cancer upon detection that the concentration of MAST4 and the concentration of the at least one other biomarker is higher in the second fluid sample than the first fluid sample. In some aspects, the method also comprises normalizing the concentration of MAST4, normetanephrine, and/or α-fetoprotein in the fluid samples with a normalization agent (for example, creatinine and coprostanol).

In certain embodiments of the aforementioned methods, the concentration of the at least one other biomarker is detected using a combination of liquid chromatography and mass spectrometry. In particular implementations, the concentration of the at least one biomarker is detected using liquid chromatography and tandem mass spectrometry.

Further disclosed herein are methods of tracking health and prosperity parameters in a population, ranging in scale from a single dwelling, a hospital, an entire city, a nation, a continent, and the world. In some aspects, the health and prosperity parameters are sustainable development goals (SDGs) established by UN members. In particular embodiments, the health and prosperity parameters are selected from the group consisting of: prevalence of undernourishment; tuberculosis incidence; malaria incidence; mortality rate attributed to cardiovascular disease, cancer, diabetes, or chronic respiratory disease; suicide mortality rate; coverage of interventions (pharmacological, psychosocial and rehabilitation and aftercare services) for substance use disorders; limit the uses of the alcohol per capita consumption; mortality rate attributed to household and ambient air pollution; mortality rate attributed to unsafe water, unsafe sanitation and lack of hygiene; mortality rate attributed to unintentional poisoning; limit the use of the tobacco and tobacco-related products; percentage of the vaccination people in national program; percentage of bloodstream infections due to selected antimicrobial-resistant organisms; access to basic sanitation facility with soap and water; improve the quality of the domestic and industrial wastewater flow; percentage of material footprint, material footprint per capita, and material footprint per GDP; documentation on (a) hazardous waste generated per capita and (b) proportion of hazardous waste treated by type of treatment; control the plastic debris density; and number of victims of intentional homicide per 100,000 population.

In yet another aspect, a method of measuring exposure to volatile organic compounds (VOCs) in a population is described herein. The method comprises compositing a wastewater from the population to provide a wastewater sample; and detecting the concentration of at least one VOC analyte in the wastewater sample. Also described are a method of tracking exposure to VOCs in a population. The method comprises providing a plurality of wastewater samples from the population over a time period, wherein the plurality of wastewater samples is composited wastewater from the population; and detecting the concentration of at least one VOC analyte in the plurality of wastewater samples. The population ranges in scale from a single dwelling, a hospital, an entire city, a nation, a continent, and the world.

In some implementations, the methods further comprise normalizing the concentration of the at least one VOC analyte in the wastewater sample with at least one normalization agent. In some aspect the at least one normalization agent comprises cotinine, wherein the method comprises normalizing the concentration of the at least one VOC analyte in the wastewater sample for nicotine consumption variability. In some implementation, detecting the concentration of the at least one VOC analyte comprises separating components of the wastewater sample and detecting the separated components of the wastewater sample using a detector. The components of the wastewater sample may be separated using liquid chromatography (LC) or gas chromatography (GC). In some aspects, the detector is selected from the group consisting of: mass spectrometry (MS), flame ionization detection (FID), and electron capture detection (ECD). In certain embodiments, the method comprises spiking the wastewater sample or composited wastewater sample with a mixture of stable isotope-labeled international standards to produce a spiked wastewater sample and diluting the spiked wastewater sample prior to detecting the concentration of at least one VOC analyte using the at least one quantification method.

In some aspects, described methods measures and tracks exposure to at least one VOC selected from the group consisting of: acrolein, acrylamide, acrylonitrile, ethylene oxide/vinyl chloride, anabasine, anatabine, benzene, 1-bromopropane, 1,3-butadiene, carbon disulfide, crotonaldehyde, cyanide, N,N-dimethylformamide, ethylbenzene, nicotine, propylene oxide, styrene, tetrachloroethylene, toluene, trichloroethylene, and xylene. In some aspects, the at least one VOC analyte is selected from the group consisting of: N-acetyl-S-(2-carboxyethyl)-L-cysteine (2CoEMA), N-acetyl-S-(3-hydroxypropyl)-L-cysteine (3HPMA), N-acetyl-S-(2-cyanoethyl)-L-cysteine (2CyEMA), N-acetyl- S-(3,4-dihydroxybutyl)-L-cysteine (34HBMA), N-acetyl-S-(4-hydroxy-2-buten-1-yl)-L-cysteine ((E)-4HBeMA), N-acetyl-S-(3-hydroxypropyl-1-methyl)-L-cysteine (3HMPMA), N-acetyl-S-(N-methylcarbamoyl)-L-cysteine (MCaMA), phenylglyoxylic acid (PhGA), nicotine (NIC), cotinine (COT), 3-hydroxycotinine (3HC), N-acetyl-S-(2-hydroxypropyl)-L-cysteine (2HPMA), N-acetyl-S-(1-phenyl-2-hydroxyethyl)-L-cysteine (2HPhEMA), N-Acetyl-S-(trichlorovinyl)-L-cysteine (122CVMA), N-acetyl-S-(benzyl)-L-cysteine (BzMA), 2-methylhippuric acid (2MHA), 3-methylhippuric acid (3MHA), and 4-methylhippuric acid (4MHA).

### BRIEF DESCRIPTION OF THE DRAWINGS

FIG. 1 depicts, in accordance with certain embodiments, a schematic of how wastewater-based epidemiology methods may be used to assess population exposure to harmful plastic constituents, even at a neighborhood level, via detection of human metabolite of plastic exposure in wastewater.

FIGS. 2A and 2B shows concentrations (FIG. 2A) and population-normalized mass loadings (PNMLs) (FIG. 2B) of phthalates, bisphenols, and terephthalic acid detected in community wastewater (n=35) in a city of the southwestern US state over a week in February 2022.

FIG. 3 shows exemplary chromatograms of analytes detected in community wastewater using isotope dilution tandem mass spectrometry. The respective chromatograms of analytes in a standard solution of 5 μg/l concentration are shown in the inset.

FIGS. 4A-4D illustrate the burden of cardiovascular disease and cancer in the United States. FIG. 4A shows the top 10 deadly diseases in the United States in 2020 shown as number of deaths. FIG. 4B shows the annual estimated economic burden of CVD and cancer in billions of U.S. Dollars (CDC, 2022b). FIG. 4C shows the number of new cancer cases per year for breast (striped pink), liver (solid red), and prostate (dotted blue) cancer (ACS, 2022a). FIG. 4D shows the number of heart attacks per year showing first-time attacks (dotted yellow) and two or more (recurring) attacks (striped grey) (CDC, 2022a).

FIG. 5 depicts, in accordance with certain embodiments, a schematic demonstrating the current individualized diagnostic model for chronic disease, shown with this study's proposition to integrate wastewater-based epidemiology as a population-level health assessment where continuous and passive screening for biomarkers indicative of disease at population-scale could serve to inform targeted interventions that can lead to early detection and potential remission of disease and the associated healthcare and societal burden.

FIGS. 6A and 6B are horizontal bar plots showing the number of countries represented from each source of data (FIG. 6A) and available data on a number of countries for each socioeconomic and health indicator used in this study (FIG. 6B).

FIGS. 7A-7H depict visual representation of the coverage of the number of municipal wastewater treatment plants (number, A), people served by centralized wastewater treatment plants (percentage, FIG. 7B), GDP per capita (US dollars per person, FIG. 7C), life expectancy at birth (years, FIG. 7D), DALYs—attributed to diarrheal diseases (years per thousand, FIG. 7D), mortality due to unsafe water, sanitation and hygiene (number per million, FIG. 7F), least basic sanitation services (percentage, FIG. 7G), and mortality rate due to unintentional poisoning (number per million, FIG. 7H).

FIGS. 8A-8D are correlation heat map of the percentage of people connected to wastewater treatment with: GDP per capita, life expectancy at birth, DALYs—due to diarrheal diseases, mortality due to unsafe water, sanitation, and hygiene, people with least basic sanitation, and mortality due to unintentional poisoning in various brackets of nations (High-income countries (FIG. 8A), Upper-middle income count (FIG. 8B), Low-middle income countries (FIG. 8C), and Low-income countries (FIG. 8D)).

FIGS. 9A and 9B respectively show concentrations and mass loadings of target chemicals indicative of VOCs exposure in raw wastewater (n=8) collected from the two US communities during May 2021 (Y-axes in log scale).

FIGS. 10A-10D compare of concentrations (FIGS. 10A and 10B), and population normalized mass loadings (FIGS. 10C and 10D) of VOC metabolites in raw wastewater from two US communities (Y-axes in log scale).

FIG. 11 depicts, in accordance with certain embodiments, cotinine normalized mass loads of VOCs per 1000 people in raw wastewater. Community 1 is in closer proximity to manufacturing facilities than Community 2.

FIG. 12 depicts, in accordance with certain embodiments, exemplary chromatograms of analytes detected in community wastewater using isotope dilution tandem mass spectrometry. The respective chromatograms of analytes in a standard solution of 0.75 μg L-1 concentration are shown in the inset.

FIG. 13 depicts, in accordance with certain embodiments, an exemplary schematic of wastewater-based epidemiology methods may be used to estimate community exposure to air pollution.

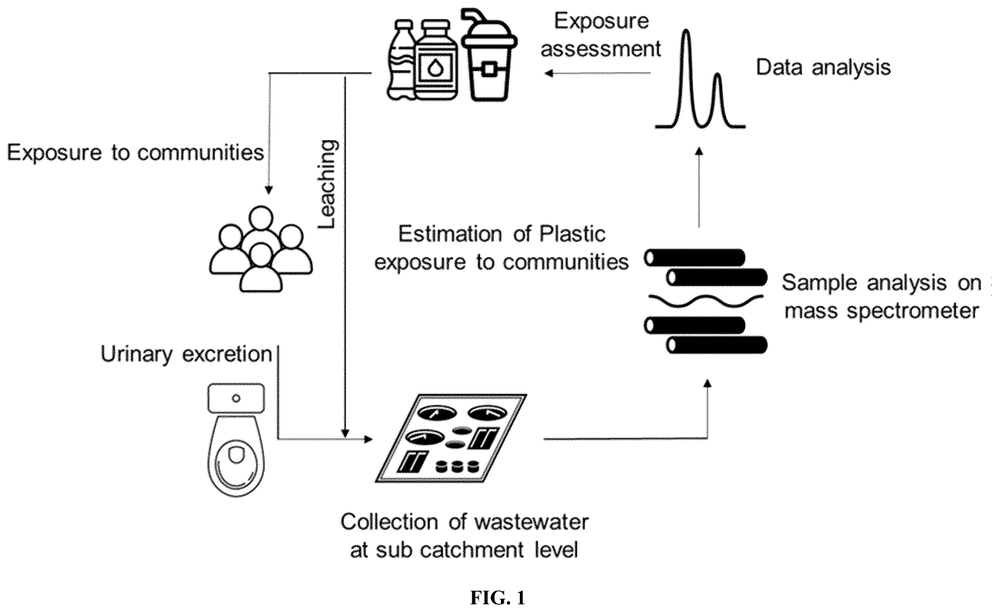

### DESCRIPTION OF THE INVENTION

Detailed aspects and applications of the invention are described below in the drawings and detailed description of the invention. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth to provide a thorough understanding of the various aspects of the invention. It will be understood, however, by those skilled in the relevant arts, that the present invention may be practiced without these specific details. It should be noted that there are many different and alternative configurations, devices, and technologies to which the disclosed inventions may be applied. The full scope of the inventions is not limited to the examples that are described below.

The singular forms “a,” “an,” and “the” include plural referents unless the context clearly dictates otherwise. Thus, for example, reference to “a step” includes reference to one or more of such steps.

As used herein, the term “wastewater sample” refers to a liquid sample collected from a wastewater treatment plant (for example, wastewater treatment plant influent or primary sludge) as well as liquid sample collected from toilets of a household or building, including from a sewer collection system or sewer infrastructure. In some aspects, a wastewater sample contains human waste, which includes urine, stool, sweat, saliva, sputum, and/or blood. Accordingly, in some aspects, the term “wastewater sample” includes sewage sludge.

Disclose herein is a method of assessing a population's health using wastewater-based epidemiology methods. In one aspect, the method assesses human exposure to plasticizers and plastic monomers at a population level. In another aspect, the method assesses a population's risk for and prevalence of cardiovascular disease and/or cancer. In yet another aspect, the method measures or tracks a population's exposure to volatile organic compounds (VOCs). In still another aspect, the method tracking health and prosperity at a population level, which can be used to track progress on the United Nation's Sustainable Development Goals (SDGs). The population may be the global population or a country's population or be on a smaller scale, such as a city's population, a neighborhood's population, or a building's population.

Wastewater-based epidemiology (WBE) has been successfully implemented for a wide variety of applications, such as monitoring licit and illicit substances (i.e., alcohol, pharmaceuticals, opioids) and, more recently, severe acute respiratory syndrome coronavirus 2 (SARS-CoV-2) during the COVID-19 pandemic. WBE is a comprehensive, cost-effective, and rapid technique that can provide essential qualitative or quantitative information on residents' health and behavior within a given wastewater catchment area via the detection of urinary and fecal markers in composited municipal sewage.

Due to its capability of monitoring community-wide trends of human activity, behavior, and exposure, WBE has been identified as an efficient and cost-effective method for obtaining population-level human health information, while still preserving individual privacy. Indeed, WBE has been demonstrated in the past to successfully function as an early warning system that can detect community-level human health biomarkers and preclude clinical manifestations of such, including poliovirus and hepatitis A and B, that was then later proved to be true once again during the COVID-19 pandemic. There is one report to date that performed a suspect screening approach to determine feasibility for measuring select anti-cancer therapeutic agents such as inositol, megestrol, and viridiflorine, using a WBE approach, suggesting a path forward for future investigation and indicating the field has already begun to respond to the detriment of these deadly diseases. Several other studies have mentioned the use and potential benefits of WBE for monitoring chronic diseases at population-scale. Thus, the methodology not only lends itself as a viable threat-monitoring tool, but also can be applied as a near real-time indicator of intervention efficacy that is inclusive of all community members in order to acquire community-level health information.

The currently practiced routine tracking of SARS-CoV-2 and illicit drugs in wastewater by contemporary WBE was preceded by pioneering work on typhoid fever in Ireland and England in the 1920s and poliomyelitic viruses in the US in 1939. Today, WBE is applied much more broadly for assessing the usage of pharmaceuticals and personal care products, illicit drugs, tobacco, and alcohol intake, as well as for determining exposure to and excretion of a spectrum of infectious disease agents, antibiotic resistance genes, industrial chemicals, endogenous hormones, and biomarkers of nutritional status. To date, few reports exist that perform experimental investigation on endogenous human biomarkers, such as examining community stress through the hormones' cortisone and cortisol, offering a great deal of opportunity for potential future investigation and experimentation.

The method described herein comprises collecting wastewater at a neighborhood level and determining the concentration of a targeted analyte from urine, such as plasticizer metabolites (also referred to herein as “plastic components”), disease biomarkers, health condition biomarkers, or other environmental factors. In some implementations, the method comprises determining the concentration of at least one analyte in the wastewater sample, wherein the at least one analyte is selected from the group consisting of: a hunger hormone, a stress hormone, cardiovascular disease biomarker, pulmonary disease biomarker, cancer biomarker, illicit drug, personal care product, surfactant, hazardous chemical, disease resistant pathogen, an antimicrobial resistance gene, a psychotropic drug, an alcohol-related chemical biomarker, a metabolite of VOCs, a nicotine-related metabolite, a plastic component, and endogenous mental health marker.

The hunger hormone may be ghrelin. The stress hormone may be selected from cortisone and cortisol. The pulmonary disease biomarker may be desmosine. The illicit drug may be selected from fentanyl, heroin, and cocaine. The personal care product may be selected from parabens, triclosan, and microbeads. The surfactant may be selected from benzalkonium chloride and benzethonium chloride among other commonly used surfactant. The hazardous chemical may be selected from arsenic, benzene, lead, and mercury and may be an environmental toxin. The psychotropic drug may be selected from alprazolam, tricyclic antidepressants, Z-drugs, and NPS. The alcohol-related chemical marker may be ethyl sulfate. The nicotine-related metabolite may be selected from cotinine and nornicotine.

The cardiovascular disease biomarker includes endogenous and exogeneous biomarkers related to cardiovascular diseases (such proteinuria, collagen fragments) as well as a biomarker selected from cystatin C and cardiac troponin I (cTnI).

The cancer biomarker may be selected from psoriasin, lipocalin-2 nucleoside, microtubule assisted serine/threonine kinase 4 (MAST4), normetanephrine, and α-fetoprotein.

The metabolite of VOCs is a metabolite of a VOC selected from the group consisting of: acrolein, acrylamide, acrylonitrile, ethylene oxide/vinyl chloride, anabasine, anatabine, benzene, 1-bromopropane, 1,3-butadiene, carbon disulfide, crotonaldehyde, cyanide, N,N-dimethylformamide, ethylbenzene, nicotine, propylene oxide, styrene, tetrachloroethylene, toluene, trichloroethylene, and xylene or is listed in Table 13. In some implementations, the at least one VOC metabolite is selected from the group consisting of: N-acetyl-S-(2-carboxyethyl)-L-cysteine (2CoEMA), N-acetyl-S-(3-hydroxypropyl)-L-cysteine (3HPMA), N-acetyl-S-(2-cyanoethyl)-L-cysteine (2CyEMA), N-acetyl-S-(3,4-dihydroxybutyl)-L-cysteine (34HBMA), N-acetyl-S-(4-hydroxy-2-buten-1-yl)-L-cysteine ((E)-4HBeMA), N-acetyl-S-(3-hydroxypropyl-1-methyl)-L-cysteine (3HMPMA), N-acetyl-S-(N-methylcarbamoyl)-L-cysteine (MCaMA), phenylglyoxylic acid (PhGA), nicotine (NIC), cotinine (COT), 3-hydroxycotinine (3HC), N-acetyl-S-(2-hy droxypropyl)-L-cysteine (2HPMA), N-acetyl-S-(1-phenyl-2-hydroxyethyl)-L-cysteine (2HPhEMA), N-Acetyl-S-(trichlorovinyl)-L-cysteine (122CVMA), N-acetyl-S-(benzyl)-L-cysteine (BzMA), 2-methylhippuric acid (2MHA), 3-methylhippuric acid (3MHA), and 4-methylhippuric acid (4MHA).

The plastic component may be plasticizer or a plastic monomer. In some aspects, the plastic component is selected from the group consisting of bisphenol A (BPA), BPA monosulfate, bisphenol S, monobenzyl phthalate (MBzP), mono (2-ethyl-5-hydroxyhexyl) phthalate (MEHHP), mono-2-ethylhexyl phthalate (MEHP), monoethyl phthalate (MEP), monobutyl phthalate (MMP), mono-n-octyl phthalate (MnOP), TPA, monobutyl phthalate (MBP), monoisobutyl phthalate (MiBP), and mono-(2- ethyl-5-oxohexyl) phthalate (MEOHP).

In addition to identifying potentially viable candidates for detection in wastewater, there are other considerations that are pertinent for downstream analysis and data interpretation as it applies in this context of endogenous and human-excreted biomarkers. When performing WBE both traditionally and in its more current state, untreated wastewater samples consisting of composited excreted contributions (i.e., urine, blood, feces, sputum) from human populations are collected at a wastewater treatment plant (WWTP) (city-level), from within the sewer collection system (neighborhood-level), or in a smaller community setting, such as a university campus (near-source or building-level). Sample collection from within the sewer infrastructure or at the building-level would likely serve to enhance detectability of human-excreted endogenous compounds given their potential vulnerability to in-sewer degradation. Regardless, wastewater is a composited fluid containing various amounts of human waste. Thus, in order to relate the detection of a marker to a person or number of people in a population, it would be advantageous to normalize the signal from a biomarker to the number of people from which it originates. Normalizing to other compounds that are associated with certain diseases or health state, such as pharmaceuticals typically prescribed for treatment of the disease or condition as well as risk factors for contributing to the disease or condition (for example, smoking, alcohol use, or poor dietary behaviors) further crystallize downstream data interpretation. Thus, in certain implementations, the described method further comprises normalizing the concentration of the at least one analyte in the fluid sample with a normalization agent. In some aspects, the normalization agent may be creatinine, coprostanol, pepper mild mottle virus, or cotinine.

Alternative normalization agents may be identified by using peaks of mass spectrometry from wastewater analysis that originate from human waste and that attenuate at the specific rate of a given biomarker of interest. For example, the steps for identifying a suitable normalization agent for a particular analyte within a particular wastewater sample comprises (1) showing presence of the analytical peak in human waste and absence or negligible amounts in rainwater, stormwater and graywater; (2) measuring the decay of a human biomarker in wastewater over time in the lab or in the field; (3) collecting mass spectra from scanning analysis of wastewater to determine human waste specific peaks that accentuate at a rate similar or identical to the human biomarker of interest; (4) optionally using artificial intelligence or machine learning tools to determine the best single or combination of mass spectrometry peaks (panel) for use as a normalization agent; and (5) normalizing the mass of a biomarker detected to the population size from which it originates by following mathematical corrections known by those skilled in the art (for example, those disclosed in Reynolds et al., “Coprostanol as a Population Biomarker for SARS-CoV-2 Wastewater Surveillance Studies”, Water, 2022, 14, 225).

In some implementations, the concentration of the at least one analyte is determined using at least one method selected from the group consisting of: liquid chromatography (LC), gas chromatography (LC), mass spectrometry (MS, including high resolution MS, HRMS), flame ionization detection (FID), and electron capture detection (ECD). In some implementations, the concentration of the at least one biomarker is determined using a combination of GC and another method, for example, GC-MS, GC-FID, or GC-ECD. In particular embodiments, the concentration of the at least one biomarker is determined using a combination of LC and MS, for example, LC-MS or LC-HRMS.

In some embodiments, the fluid sample comprising human waste is collected from a sewage infrastructure, for example, a residential sewershed. In some aspects, the fluid sample collected a sewer infrastructure is composited, for example, to a state or province and the population is the state's or province's population; to a city level and the population is the city's population; to a neighborhood level and the population is a neighborhood; to a city block and the population is the population found inside the city block; to a building level and the population is a building's occupants; or a household level and the population is a household. In certain implementations, the fluid sample collected a sewer infrastructure is composited, for example, to a national, continental, or global level. In some aspects, the fluid sample is frozen after collecting until sample processing, wherein the thawed fluid sample is then processed. In certain implementations, the fluid sample is processed within two weeks, within ten days, within a week, or within five days of collection. In certain implementations where the analyte are certain compounds, the wastewater sample is spiked with a mixture of stable isotype-labeled international standards to produce a spiked wastewater sample, and the method further comprises diluting the spiked wastewater sample prior to detecting the concentration of chemical analyte using at least one quantification method described in the previous paragraph.

As many WBE studies are commonly performed on a longitudinal basis, with sample collection occurring at a regular cadence, this method can ultimately serve to complement the traditional medical model of ‘grab sampling’ individual patients, as these time-discrete samples only reflect the health status at one point in time from a single person. Thus, a great benefit to WBE is the ability to exhibit trends over time of all individuals within a given community, not just those who are able to seek regular health screenings by a physician, offering unique insights that would then prompt relevant public health interventions when necessary; ultimately adopting a diagnostic approach for public health. In such implementations, the method of assessing a population's heath comprises providing a first wastewater sample from the population and providing a second wastewater sample from the population, wherein the second fluid sample is collected from a same location as the first wastewater sample and is collected at least two days after collecting the first wastewater sample. The method further comprises detecting the concentration of at least one analyte in the first wastewater sample and the second wastewater sample, wherein the at least one analyte is selected from the group consisting of: a hunger hormone, a stress hormone, cardiovascular disease biomarker, pulmonary disease biomarker, cancer biomarker, illicit drug, personal care product, surfactant, hazardous chemical, disease resistant pathogen, an antimicrobial resistance gene, a psychotropic drug, an alcohol-related chemical biomarker, a metabolite of volatile organic compounds (VOCs), an environmental toxin, a nicotine-related metabolite, a plastic component, and endogenous mental health marker; and detecting the concentration of at least one analyte in the second fluid sample. The method then comprises comparing the concentrations of the at least one analyte in the wastewater samples and identifying a change in the population's health upon detection that the concentration of the at least one biomarker is changed between the wastewater fluid sample and the second wastewater sample.

Also described herein are a method for identifying analytes for wastewater-based assessments of a population's health. The method comprises evaluating scientific literature reporting correlations between an analyte and biological state (for example, a disease or condition or exposure to an environmental toxin). The evaluation utilizes the Bradford Hill criteria to infer causal relationships through epidemiological analysis. Exemplary evaluation of scientific literature is described in Example IX (subsection a) and Example XIII. In some aspects, the candidate analytes for wastewater analysis identified from evaluating scientific literature are then evaluated to confirm feasibility of detection in wastewater samples and that the concentrations changes in the analytes could also be detected.

### Plasticizers and Plastic Monomers

The ubiquity of plastics in the laboratory environment and in analytical instrumentations and supplies create a challenge in laboratory assessment of plasticizer exposure. The plastic-made sampling containers may also serve as a source of elevated background levels of phthalates. However, minimal interference from the bottle itself would be expected if collected samples were immediately brought to the lab and quickly processed, to limit the wastewater storage time in the plastic container. It has been documented in the literature that the leaching of plastic chemical constituents depends on several factors, including the period of storage of liquid inside the plastic container. However, as shown in the Examples, urinary biomarkers of human exposure to plastic monomers and additives could be detected in community wastewater at the neighborhood level, especially in the United States.

The method of assessing human exposure to plasticizers and plastic monomers at a population level described herein comprises providing a fluid sample comprising human waste from the population and determining the concentration of at least one analyte selected from the group consisting of: bisphenol A (BPA), BPA monosulfate, bisphenol S, monobenzyl phthalate (MBzP), mono (2-ethyl-5-hydroxyhexyl) phthalate (MEHHP), mono-2-ethylhexyl phthalate (MEHP), monoethyl phthalate (MEP), monobutyl phthalate (MMP), mono-n-octyl phthalate (MnOP), TPA, monobutyl phthalate (MBP), monoisobutyl phthalate (MiBP), and mono-(2- ethyl-5-oxohexyl) phthalate (MEOHP) in the fluid sample. In some aspects, the at least one analyte is MEHP.

In one aspect, the method of assessing human exposure to plasticizers and plastic monomers at a population level comprises collecting wastewater at a neighborhood level and determining the concentration of plasticizer metabolites in urine. In some aspects, the methods assess exposure to phthalates, bisphenols (BPs), and terephthalic acid (TPA) by measuring urinary metabolites in community wastewater.

In certain implementations, the method of assessing human exposure to plasticizers and plastic monomers at a population level comprises further comprises normalizing the concentration of the at least one analyte in the fluid sample with a normalization agent. The normalization agent may be creatinine and/or coprostanol.

In some aspects, the method of assessing human exposure to plasticizer and plastic monomers in a population comprises collecting a fluid sample comprising human waste from the population; freezing the collected fluid sample; and determining the concentration of at least one plasticizer or plastic monomers analyte in the fluid sample within two weeks of collecting the fluid sample. The collected fluid sample is thawed to determine the concentration of the at least one plasticizer or plastic monomers analyte.

### Cardiovascular Disease and Cancer

Given the degree of morbidity, mortality, and healthcare burden experienced within the United States, the identification of biomarkers for wastewater-based monitoring of cardiovascular disease (CVD) and cancer is useful. While biomarker discovery and medical detection of diseases such as CVD and cancer are continuously expanding, prevalence rates in the United States and globally continue to exhaust the efforts of medical and public health professionals. Promptness in disease detection and monitoring of disease prevalence are key for improving global health. Compared to clinical investigation, WBE is often viewed as a rapid and cost-effective source of human health information that can fully operate without interrupting the day-to-day activities of the community.

WBE can be used in concert to offer a comprehensive view of the disease burden in populations on a more inclusive spectrum; those that are disease inflicted yet asymptomatic, and those without access to healthcare facilities or insurance. These benefits of being a rapid, inclusive, and non-invasive methodology for acquiring community-level and actionable data were highlighted early on during the COVID-19 pandemic when clinical surveillance was lacking, and viral spread continued at an exponential pace. FIG. 5 proposes the incorporation of WBE alongside individualized diagnostics. More research investigating long-term impacts of COVID-19 support strong connections between post-infection and increased risk of incident cardiovascular events, such as stroke (hazard ratio (HR) 1.52; 95% CI 1.43, 1.62), heart failure (hazard ratio (HR) 1.72; 95% CI 1.65, 1.80), and dysrhythmias (hazard ratio (HR) 1.69; 95% CI 1.64, 1.75). Thus, it is not only timely but imperative to begin investigating the use of WBE to understand these trends in CVD-related events.

As shown in the Examples, monitoring cardiac troponin I (cTnI), cystatin C, α-fetoprotein, normetanephrine, and (MAST4) using WBE can provide a cost-effective, non-invasive, and inclusive method of collecting substantial amounts of population-level data regarding the occurrence of CVD and/or cancer all while preserving community anonymity. Elevated levels of α-fetoprotein can indicate either prostate or liver cancer, while cystatin C has also been shown to increase in patients with renal failure from atherosclerosis. While these proposed biomarkers would indicate CVD and cancer prevalence overall, it would be beneficial to identify specific types or subsets of biomarkers for downstream data analysis at the population scale. Compounding wastewater-derived data of endogenous markers with measurements of related pharmaceuticals or relevant external data sets could transform the overall disease prevalence relationship to the scale of a particular population scale, such as a neighborhood level or city level.

Thus, normalizing the disease-related biomarkers to other compounds that are associated with these diseases (pharmaceutical treatments or analytes related to the disease's risk factor) crystallizes downstream data interpretation. For cancer, this could also be applied through measurements of chemotherapy or other therapeutic intervention drugs as 75% of the procedures are outpatient, thus the human excreted markers would likely be contributed to the local wastewater collection system. For cardiovascular diseases, this could be pharmaceuticals typically prescribed for treatment of these diseases and risk factors such as smoking, alcohol use, or poor dietary behaviors.

For the identified biomarkers that have been shown to peak during the first-morning urine, such as elevated cTnI that can indicate an upcoming cardiovascular event, this could also inform new strategical approaches for time-targeted wastewater sample collection in order to enhance probability of detecting a signal while also providing near real-time, actionable data. Further, instrumentation commonly used for chemical analysis by WBE to understand community-level metabolomics has largely involved liquid chromatography-tandem mass spectrometry (LC-MS/MS), while biological analyses has leveraged genomic analyses including quantitative PCR (qPCR, RT-qPCR) or sequencing (shotgun, next generation, etc.) in response to the COVID-19 pandemic. A new frontier for WBE is the recruitment of proteomics in order to continue to advance the field and inform on other unique aspects of human health, behavior, exposure, and activity. Few studies have begun exploring this novel avenue which will serve as a foundation to inform future work; including the detection of the herein proposed CVD and cancer biomarkers that include a wide range of low to high molecular weight proteins (Table 7).

While WBE has been identified here to potentially represent a powerful tool in the tracking and monitoring of global deadly diseases, there are certain limitations that should be considered. For example, endogenous biomarkers are commonly excreted in much lower concentrations, rendering detection and confident quantification more challenging. Mixing of urine with stool also injects uncertainty as to the absolute quantity of biomarkers present and makes the detection of urinary biomarkers more difficult due to increased matrix effects of fecal matter. Furthermore, biomarkers may be fairly stable in urine but much more susceptible to degradation or transformation within the sewerage system, due to the presence of fecal microbiomes and active biofilms covering the inner linings of sewer pipes. Attempting to circumvent this issue by collecting samples closer to the point of contribution may enhance detection abilities, but simultaneously may raise ethical concerns due to the possibility of potential identification and stigmatization of affected sub-populations or even individuals.

For methods of diagnosing cardiovascular disease and/or cancer using a fluid sample containing urine or for assessing the prevalence of CVD and/or cancer in a population, the method comprises providing a fluid sample comprising urine from the population (for example, a wastewater sample) and detecting the concentration of at least one biomarker selected from the group consisting of: cardiac troponin I (cTnI), cystatin C, normetanephrine, α-fetoprotein, and microtubule assisted serine/threonine kinase 4 (MAST4), in the fluid sample. The method further comprises identifying the population as containing a significant portion of subjects with CVD and/or cancer based on the concentration of the at least one biomarker. In some aspects, the population contains a significant portion of subjects with CVD and/or cancer when the concentration of the at least one biomarker is at least 1.0×10⁻¹ ng/L/10,000 population. In some aspects, the concentration of the at least one biomarker is at least 2.0×10⁻¹ ng/L/10,000 population, at least 1.2×10³ ng/L/10,000 population, or at least 6.9×10³ ng/L/10,000 population.

In some aspects of the method of assessing the prevalence of CVD and/or cancer in a population, the method comprises providing a first fluid sample comprising urine from the population, providing a second fluid sample comprising urine from the population, and comparing the change in the concentration of at least one at least one biomarker selected from the group consisting of: cTnI, cystatin C, normetanephrine, α-fetoprotein, and MAST4 between the first fluid sample and the second fluid sample. In some aspects, the first fluid sample and the second fluid sample are collected from a same source and the second fluid sample is collected at least two days after the first fluid sample. The method further comprises identifying the population has increased incidence of CVD and/or cancer when the concentration of the at least one at least one biomarker is higher in the second fluid sample. In some aspects, the concentration of the at least one at least one biomarker is higher in the second fluid sample is at least 1.0×10⁻¹ ng/L/10,000 population, at least 2.0×10⁻¹ ng/L/10,000 population, at least 1.2×10³ ng/L/10,000 population, or at least 6.9×10³ ng/L/10,000 population.

The described method may also be used to diagnose a subject as having CVD and/or cancer. In such implementation, the fluid sample may be, for example, a urine sample, and the subject is diagnosed as having CVD and/or cancer when the concentration of the at least one biomarker is at least 1.0×10⁻¹ ng/L. In some implementations, the fluid sample may be a wastewater sample from the subject.

### United Nations Sustainable Development Goals

With the rapid urbanization and evidence of climate change, the member countries of the United Nations (UN) have unanimously agreed on agendas for attaining sustainable social, economic, and environmental development milestones. With a target date of 2030, these agendas comprise 17 different sustainable development goals (SDGs). A common criticism, however, of the UN agenda is the difficulty in tracking these goals, which are framed as qualitative parameters.

As shown in the Examples, there are analytes in wastewater that can shed light on health and prosperity status within a population, which can be used to track progress on the UN's SDGs. WBE is an inexpensive and practical tool for monitoring dietary intake and dietary deficiencies in populations around the world and for the study of endogenous biomarkers, such as stress hormones that may provide insights into human wellbeing and quality of life. Thus, among the 17 different sustainable development goals listed by the UN 2030 agenda, more than half of these may be monitored by using WBE monitoring at centralized treatment infrastructure. Table 10 lists the biomarkers for track health and prosperity in a population, organize by the corresponding SDG.

The 9 different SDGs identified include: SDG#2 which aims to “end hunger, achieve food security and improved nutrition and promote sustainable agriculture”, SDG #3 to “ensure healthy lives and promote well-being for at all ages”, SDG #6 to “ensure availability and sustainable management of water and sanitation for all”, SDG #8 to “promote sustained, inclusive and sustainable economic growth, full and productive employment and decent work for all”, SDG #9 to “build resilient infrastructure, promote inclusive sustainable industrialization and foster innovation”, SDG #11 to “make cities and human settlements inclusive, safe, resilient and sustainable”, SDG #12 “ensure sustainable consumption and production patterns”, SDG #14 “conserve and sustainably use the oceans, seas and marine resources for sustainable development”, and SDG #16 “promote peaceful and inclusive societies for sustainable development, provide access to justice for all and build effective, accountable and inclusive institutions at all levels”.

The reviewed literature (from years 2005 to 2021) identified 25 different classes of endogenous and exogeneous biomarkers as shown in Table 10, that can be used in tracking progress towards the attainment of the SDGs. These biomarkers include hunger hormones (e.g., ghrelin), stress hormones (e.g., cortisone, cortisol), endogenous and exogeneous biomarkers related to cardiovascular diseases (e.g., proteinuria, collagen fragments), pulmonary diseases (e.g., desmosine), cancer (e.g., psoriasin, lipocalin-2 nucleosides, etc.), different classes of illicit drugs (e.g., fentanyl, heroin, cocaine etc.), personal care products (e.g., parabens, triclosan, microbeads) and surfactants (e.g., benzalkonium chloride, benzethonium chloride etc.), hazardous chemicals (e.g., arsenic, benzene, lead, mercury), drug resistant pathogens, antimicrobial resistant genes and psychotropic drugs (e.g., alprazolam, tricyclic antidepressants, Z-drugs, NPS etc.). All of these biosignatures may provide value for tracking progress in attaining the UN SDGs.

TABLE 10
Summary of sustainable development goal agendas and the corresponding
indicators for tracking using wastewater-based epidemiology.

| SDGs 2030 | | |
|---|---|---|
| agendas | Goal attributes | Progress tracking using WBE |
| 2.1.1 | Prevalence of undernourishment | Endogenous biomarkers of starvation (For |
| | | e.g., hunger hormones) |
| 3.3.2 | Tuberculosis incidence | Quantification of Mycobacterium |
| | | tuberculosis |
| 3.3.3 | Malaria incidence | Quantification of Plasmodium parasites |
| 3.4.1 | Mortality rate attributed to | Endogenous and Exogeneous biomarkers |
| | cardiovascular disease, cancer, | related to heart diseases, pulmonary |
| | diabetes, or chronic respiratory | difficulties, and cancer-causing peptides |
| | disease | |
| 3.4.2 | Suicide mortality rate | Stress metabolites and psychotropic drugs |
| | | as proxies of vulnerabilities |
| 3.5.1 | Coverage of treatment | Study of consumption of illicit and |
| | interventions (pharmacological, | prescribed psychotropic drugs |
| | psychosocial and rehabilitation | |
| | and aftercare services) for | |
| | substance use disorders | |
| 3.5.2 | Limit the uses of the alcohol per | Alcohol related chemical biomarkers (E.g., |
| | capita consumption | Ethyl Sulfate) |
| 3.9.1 | Mortality rate attributed to | Metabolites of volatile organic compounds |
| | household and ambient air | as vulnerabilities |
| | pollution | |
| 3.9.2 | Mortality rate attributed to unsafe | Tracking susceptibility by quantifying |
| | water, unsafe sanitation, and lack | pathogens and related prescribed drugs and |
| | of hygiene (exposure to unsafe | biomarkers |
| | Water, Sanitation and Hygiene | |
| | for All (WASH) services) | |
| 3.9.3 | Mortality rate attributed to | Environmental toxins (For e.g., Arsenic) |
| | unintentional poisoning | |
| 3.a.1 | Limit the use of the tobacco and | Nicotine-related metabolites (for e.g., |
| | tobacco-related products | cotinine, nomnicotine etc.) |
| 3.b.1 | Percentage of the vaccination | Measuring vulnerabilities of unvaccinated |
| | people in national program | population through biomarker assessment |
| 3.d.2 | Percentage of bloodstream | Drug resistant pathogens and antimicrobial |
| | infections due to selected | resistant genes |
| | antimicrobial-resistant organisms | |
| 6.2.1 | Access to basic sanitation facility | Assessment of personal care products (For |
| | with soap and water | e.g., Surfactants) s |
| 6.3.1 | Improve the quality of the | Biological Oxygen Demand, Nitrogenous |
| | domestic and industrial | Oxygen Demand etc. |
| | wastewater flow | |
| 8.4.1 | Percentage of material footprint, | Quantification of certain materials |
| | material footprint per capita, and | |
| | material footprint per GDP | |
| 9.5.1 | Research and development | Implementation of WBE demands |
| | expenditure as a proportion of | investment in the research laboratories and |
| | GDP | manpower. |
| 9.5.2 | Researchers per million | Different applications of WBE increase the |
| | inhabitants | inflow of researchers from |
| | | transdisciplinary field. |
| 9.a.1 | Total official international | Research related to WBE expands the |
| | support | research dimensions and ultimately brings |
| | | support from international collaboration. |
| 11.6.1 | Improve the management of | Data on type of wastewater treatment |
| | municipal solid waste collected | system |
| | and managed in controlled | |
| | facilities out of total municipal | |
| | waste generated, by cities | |
| 12.4.2 | Documentation on (a) Hazardous | Quantification of chemical suites of |
| | waste generated per capita; and | hazardous materials |
| | (b) proportion of hazardous waste | |
| | treated, by type of treatment | |
| 14.1.1 | Control the plastic debris density | Identification and relative quantification of |
| | | plastics |
| 16.1.1 | Number of victims of intentional | Quantification of endogenous mental |
| | homicide per 100,000 population, | health markers as proxies of vulnerabilities |
| | by sex and age | |

Thus, in certain embodiments, the method of tracking health and prosperity in a population comprises compositing a wastewater from the population to provide a wastewater sample; and detecting the concentration of at least one analyte in the wastewater sample, wherein the at least one analyte is selected from the group consisting of: a hunger hormone, a stress hormone, cardiovascular disease biomarker, pulmonary disease biomarker, cancer biomarker, illicit drug, personal care product, surfactant, hazardous chemical, disease resistant pathogen, an antimicrobial resistance gene, and a psychotropic drug. For example, the at least one analyte is selected from the group consisting of: a hunger hormone, a stress hormone, a pathogen cardiovascular disease biomarker, pulmonary disease biomarker, cancer biomarker, illicit drug, personal care product, surfactant, hazardous chemical, disease resistant pathogen, an antimicrobial resistance gene, and a psychotropic drug. In some implementations, the wastewater is collected and/or composited from a sewage infrastructure, for example, a residential sewershed. In some aspects, the wastewater is composited to a state or province and the population is the state's or province's population; to a city level and the population is the city's population; to a neighborhood level and the population is a neighborhood; to a city block and the population is the population found inside the city block; to a building level and the population is a building's occupants; or a household level and the population is a household. In certain implementations, the fluid sample collected a sewer infrastructure is composited, for example, to a national, continental, or global level.

As wastewater is a composited fluid containing various amounts of human waste. In order to relate the detection of a marker to a person or number of people in a population, it would be advantageous to normalize the signal from a biomarker to the number of people from which it originates. Accordingly, in some implementations, the method further comprises normalizing the concentration of the at least one analyte in the wastewater sample with a normalization agent (for example, creatinine, and coprostanol, and pepper mild mottle virus). Alternative normalization agents may be identified by using peaks of mass spectrometry from wastewater analysis that originate from human waste and that attenuate at the specific rate of a given biomarker of interest.

In particular embodiments, the concentration of the at least one biomarker is determined using a combination of LC and tandem MS. Tracking the change in the concentration of the at least one analyte provides a quantitative record of changes in the health and prosperity level in a population. For example, where the concentration of the hunger hormone, stress hormone, cardiovascular disease biomarker, pulmonary disease biomarker, cancer biomarker, illicit drug, hazardous chemical, disease resistant pathogen, antimicrobial resistance gene, or a psychotropic drug in wastewater sample decreases over time, then the health and prosperity level of the population is increasing. Thus, the population is making progress towards the SDGs. In other aspects, where the concentration of the personal care product or surfactant in the wastewater sample increases over time, then the health and prosperity level of the population is increasing. Thus, the population is making progress towards the SDGs.

In certain aspects, the method of tracking health and prosperity in a population comprises providing a plurality of wastewater samples from the population over a period of time; and detecting the concentration of a hunger hormone, a stress hormone, cardiovascular disease biomarker, pulmonary disease biomarker, cancer biomarker, illicit drug, personal care product, surfactant, hazardous chemical, disease resistant pathogen, antimicrobial resistance gene, and a psychotropic drug in the plurality of wastewater samples. Where the concentration of the hunger hormone, stress hormone, cardiovascular disease biomarker, pulmonary disease biomarker, cancer biomarker, illicit drug, hazardous chemical, disease resistant pathogen, antimicrobial resistance gene, and a psychotropic drug in the plurality of wastewater samples decreases over the period of time, then the health and prosperity level of the population is increasing during the period of time. Where the concentration of the personal care product and surfactant in the plurality of wastewater samples increases over the period of time, then the health and prosperity level of the population is increasing during the period of time. Thus, the population is making progress towards the SDGs. In some aspects, the plurality of wastewater samples is composited wastewater from the population. The population may be at a household level, a building level, neighborhood level, a city level, a national level, or a continental level.

In some implementations, the method of tracking health and prosperity in a population further comprises determining the quantity of Mycobacterium tuberculosis and/or Plasmodium in the wastewater sample or plurality of wastewater samples and/or determining the concentration of an alcohol-related chemical biomarker (for example, ethyl sulfate), a metabolite of volatile organic compounds, an environmental toxin (for example, arsenic), a nicotine-related metabolite (for example, cotinine or nornicotine), a plastic components, and/or endogenous mental health markers. Decreasing quantity of M. tuberculosis and/or Plasmodium or decreasing concentration of the alcohol-related chemical biomarker, the metabolite of volatile organic compounds, the environmental toxin, the nicotine-related metabolite, or the plastic components is representative of increasing health and prosperity in the population.

The hunger hormone analyte may be ghrelin. The stress hormone analyte may be cortisone or cortisol. The biomarkers related to cardiovascular disease, pulmonary disease, and cancer may be endogenous or exogeneous. In some embodiments, the cardiovascular disease biomarker is proteinuria or collagen fragments. In some embodiments, the pulmonary disease biomarker may be desmosine. In some embodiments, cancer biomarker is psoriasin or lipocalin-2 nucleosides. The illicit drugs may be fentanyl, heroin, or cocaine. The personal care product may be parabens, triclosan, or microbeads. The surfactants may be benzalkonium chloride or benzethonium chloride. The hazardous chemicals may be arsenic, benzene, lead, or mercury. The psychotropic drugs may be alprazolam, tricyclic antidepressants, Z-drugs, and NPS.

### Volatile Organic Compounds

WBE provides the opportunity to monitor urinary metabolites of VOC exposure at the community level, bypassing the cost and invasiveness associated with individual specimen collection. A recent study indicated the cost of WBE was <1% of the cost of conventional data collection methods. Previous studies have shown various VOC metabolites excreted in the urine are amendable to mass spectrometry methods with measured concentrations ranging from <1 ng mL⁻¹ to <10,000 ng mL⁻¹ (Li et al., 2021). As shown in the Examples, WBE is a cost-effective, rapid, and practical tool for assessing air pollution exposures at the community level that do not rely on any knowledge about indoor air quality, ambient air quality, and time of residents spent outdoors.

Thus, in certain embodiments, the method of measuring a population's exposure to VOCs comprises compositing a wastewater from the population to provide a wastewater sample; and detecting the concentration of at least one analyte in the wastewater sample, wherein the at least one VOC analyte is a metabolite of a VOC. In some aspects, the at least one VOC analyte is a metabolite of at least one VOC selected from the group consisting of: acrolein, acrylamide, acrylonitrile, ethylene oxide/vinyl chloride, anabasine, anatabine, benzene, 1-bromopropane, 1,3-butadiene, carbon disulfide, crotonaldehyde, cyanide, N,N-dimethylformamide, ethylbenzene, nicotine, propylene oxide, styrene, tetrachloroethylene, toluene, trichloroethylene, and xylene. In some implementations, the at least one VOC metabolite is selected from a VOC metabolite listed in Table 13. In other aspects, implementations, the at least one VOC analyte is a metabolite of at least one VOC selected from the group consisting of: acrolein, acrylonitrile, 1,3-butadiene, crotonaldehyde, N,N-dimethylformamide, ethylbenzene, nicotine, propylene oxide, styrene, tetrachloroethylene, toluene, and xylene. In some implementations, the at least one VOC metabolite is selected from the group consisting of: N-acetyl-S-(2-carboxyethyl)-L-cysteine (2CoEMA), N-acetyl-S-(3-hydroxypropyl)-L-cysteine (3HPMA), N-acetyl-S-(2-cyanoethyl)-L-cysteine (2CyEMA), N-acetyl-S-(3,4-dihydroxybutyl)-L-cysteine (34HBMA), N-acetyl-S-(4-hydroxy-2-buten-1-yl)-L-cysteine ((E)-4HBeMA), N-acetyl-S-(3-hydroxypropyl-1-methyl)-L-cysteine (3HMPMA), N-acetyl-S-(N-methylcarbamoyl)-L-cysteine (MCaMA), phenylglyoxylic acid (PhGA), nicotine (NIC), cotinine (COT), 3-hydroxycotinine (3HC), N-acetyl-S-(2-hydroxypropyl)-L-cysteine (2HPMA), N-acetyl-S-(1-phenyl-2-hydroxyethyl)-L-cysteine (2HPhEMA), N-Acetyl-S-(trichlorovinyl)-L-cysteine (122C VMA), N-acetyl-S-(benzyl)-L-cysteine (BzMA), 2-methylhippuric acid (2MHA), 3-methylhippuric acid (3MHA), and 4-methylhippuric acid (4MHA).

In some implementations, the method further comprises normalizing the concentration of the at least one VOC analyte in the wastewater sample with a normalization agent (for example, creatinine, coprostanol, and pepper mild mottle virus). Alternative normalization agents may be identified by using peaks of mass spectrometry from wastewater analysis that originate from human waste and that attenuate at the specific rate of a given biomarker of interest.

In some implementations, the method for measuring a population's exposure to VOCs further comprises normalizing the concentration of the at least one VOC analyte in the wastewater sample for nicotine consumption variability. In such implementations, the concentration of the at least one VOC analyte in the wastewater sample is normalized with cotinine, which is a metabolite of nicotine that is detectable in urine and in wastewater samples. In certain embodiments, the concentration of the at least one VOC analyte is determined using a combination of LC and tandem MS. Tracking the change in the concentration of the at least one analyte provides a quantitative record of the population's exposure to VOCs.

In some implementation, detecting the concentration of the at least one VOC analyte comprises separating components of the wastewater sample and detecting the separated components of the wastewater sample using a detector. The components of the wastewater sample may be separated using liquid chromatography (LC) or gas chromatography (GC). In some aspects, the detector is selected from the group consisting of: mass spectrometry (MS), flame ionization detection (FID), and electron capture detection (ECD). In certain embodiments, the method comprises spiking the wastewater sample or composited wastewater sample with a mixture of stable isotope-labeled international standards to produce a spiked wastewater sample and diluting the spiked wastewater sample prior to detecting the concentration of at least one VOC analyte using the at least one quantification method. Thus, in a particular embodiment, the method comprises providing a sample from the population; separating components of the wastewater sample using LC or GC; detecting the separated components of the wastewater sample using a detector, wherein the detector is selected from the group consisting of: MS, FID, and electron ECD; spiking the wastewater sample with a mixture of stable isotype-labeled international standards to produce a spiked wastewater sample; diluting the spiked wastewater sample; and detecting the concentration of the metabolite of VOCs in the wastewater sample from the diluted spiked wastewater sample.

In certain aspects, the method of tracking a population's exposure to VOCs comprises providing a plurality of wastewater samples from the population over a time period; and detecting the concentration of at least one VOC analyte in the plurality of wastewater samples. In some aspects, the plurality of wastewater sample is composited wastewater from the population. The population may be at a household level, a building level, neighborhood level, a city level, a national level, or a continental level. The method of tracking a population's exposure to VOCs further comprises normalizing the concentration of the at least one VOC analyte in the wastewater sample with at least one normalization agent. In some aspects, the at least one normalization agent comprises cotinine, so the concentration of the at least one VOC analyte in the wastewater sample is normalized for nicotine consumption variability. In certain implementations, the method further comprises spiking the composited wastewater sample with a mixture of stable isotype-labeled international standards to produce a spiked wastewater sample; and diluting the spiked wastewater sample prior to detecting the concentration of at least one VOC analyte using the at least one quantification method.

### EXAMPLES

The invention is further illustrated by the following examples that should not be construed as limiting. The contents of all references, patents, and published patent applications cited throughout this application, as well as the Figures, are incorporated herein by reference in their entirety for all purposes.

### I. Study Locations and Wastewater Sampling for Assessing Plasticizer and Plastic Monomers Exposure

Raw 24-h composite community wastewater samples (n=35) were collected over a week from 5 sampling locations (A1, A2, A3, A4, A5) of a city located in Arizona on the 8-14 Feb. 2022. A1, A2, and A3 are primarily residential sewersheds with a population ranging from roughly 77,000 to 390,000, whereas A4 and A5 are predominantly commercial or industrial with a population ranging from 9000 to 17,000. A4 has no residential population but several commercial industries, including aerospace and aviation, medical device manufacturing, sustainable technology development, automotive maintenance, and repair. Some companies also provide construction, vinyl painting, flooring, and recycling services. A5 is a small catchment with restaurants, medical laboratories, and corporate offices.

The sampling details, including the number of sampling locations, flow data, and catchment population, are shown in Table 1. Wastewater flow and population assessments were provided by the municipality. The population for each sewershed was determined by combining 2010 census data and city-level employment data. The census data informs on the number of residents living in the study area, and the employment data assessments the number of people working there. Both data were required for this study because some of the sampling locations had few to no residents. The employment data was available through the Maricopa Association of Governments. Samples were collected in polyethylene (PE) bottles and transported to the lab at Arizona State University on wet ice. Samples were frozen, and sample processing began with a week of receipt.

TABLE 1
Wastewater sampling details

| Sampling | Sampling | Average community | Catchment |
|---|---|---|---|
| location | sublocations | wastewater flow (L/d) | population |
| A city in | A1 | 54,366,000 | 277,000 |
| the Southwestern | A2 | 17,836,000 | 77,000 |
| State of the US | A3 | 56,405,000 | 392,000 |
| | A4 | 6,484,000 | 17,000 |
| | A5 | 403,000 | 9,000 |

### II. Chemicals and Reagents for Assessing Plasticizer and Plastic Monomers Exposure

LC-MS (liquid chromatography-mass spectrometry) grade methanol, acetonitrile, isopropanol, and water were purchased from Fisher Scientific (Thermo Fisher Scientific, Waltham, MA); LC-MS grade acetic acid was purchased from Fluka Chemical Corp (Milwaukee, WI). High purity (>97%) reference standard and isotope-labeled standard solutions of the target compounds including bisphenol A (BPA), BPA-d8, bisphenol AF (BPAF), BPAF-¹³C₁₂, bisphenol S (BPS), BPS [symbol] ¹³C₁₂, di(2-ethylhexyl) phthalate (DEHP), DEHP-¹³C₄, dimethyl phthalate (DMP), DMP-d4, monobutyl phthalate (MBP), MBP-¹³C₄, monoisobutyl phthalate (MiBP), MiBP-¹³C₄, monobenzyl phthalate (MBzP), MBzP-¹³C₄, monoethyl phthalate (MEP), MEP-¹³G, mono-2-ethylhexyl phthalate (MEHP), MEHP-¹³C₄, mono (2-ethyl-5-hydroxyhexyl) phthalate (MEHHP), MEHHP-¹³C₄, mono-(2-ethyl-5-oxohexyl) phthalate (MEOHP), MEOHP-¹³C₄, mono-n-octyl phthalate (MnOP), MnOP-¹³C₄ were obtained from Cambridge Isotope Laboratories (CIL). Terephthalic acid (TPA) was purchased from Sigma Aldrich (Milwaukee, WI), BPA monosulfate sodium salt, and BPA monosulfate sodium salt d6 were purchased from Toronto Research Chemicals (Canada). A list of 32 reference standards and their respective stable isotope-labeled internal standards (SIL-IS) is given in Table 2. Stock and working solutions of individual analytes were prepared in methanol and were stored in amber glass vials with polytetrafluoroethylene (PTFE) septa at −20° C.

TABLE 2
List of analytes included in the study with their respective labeled standards.

| Analytes | Labeled standards | Structure |
|---|---|---|
| BPA | BPA d8 | 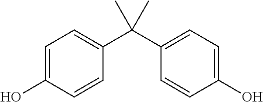 [SMILES: CC(C)(c1ccc(O)cc1)c1ccc(O)cc1] |
| BPA monosulfate | BPA monosulfate d6 | 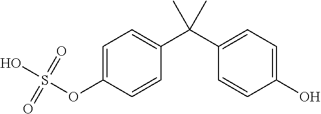 [SMILES: CC(C)(c1ccc(O)cc1)c1ccc(OS(=O)(=O)O)cc1] |
| | | bisphenol A monosulfate |
| Bisphenol AF | BPAF-¹³C₁₂ | 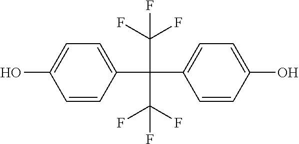 [SMILES: Oc1ccc(C(c2ccc(O)cc2)(C(F)(F)F)C(F)(F)F)cc1] |
| Bisphenol S | BPS-¹³C₁₂ | 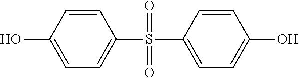 [SMILES: O=S(=O)(c1ccc(O)cc1)c1ccc(O)cc1] |
| DEHP | DEHP-¹³C₄ | 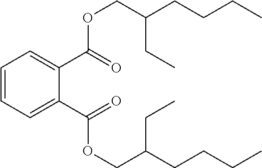 [SMILES: CCCCC(CC)COC(=O)c1ccccc1C(=O)OCC(CC)CCCC] |
| DMP | DMP-d4 | 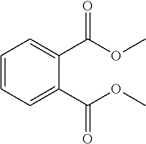 |
| MBP | MBP-¹³C₄ | 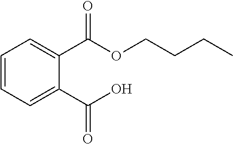 [SMILES: CCCCOC(=O)c1ccccc1C(=O)O] |
| MiBP | MiBP-¹³C₄ | 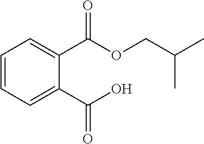 [SMILES: CC(C)COC(=O)c1ccccc1C(=O)O] |
| MBzP | MBzP-¹³C₄ | 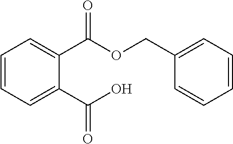 [SMILES: O=C(O)c1ccccc1C(=O)OCc1ccccc1] |
| MEP | MEP-¹³C₄ | 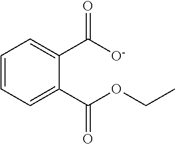 |
| MEHP | MEHP-¹³C₄ | 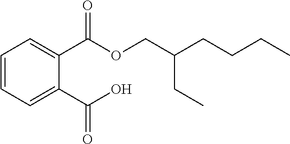 [SMILES: CCCCC(CC)COC(=O)c1ccccc1C(=O)O] |
| MEHHP | MEHHP-¹³C₄ | 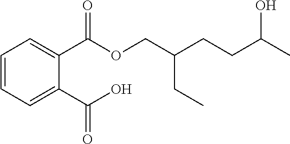 [SMILES: CCC(CCC(C)O)COC(=O)c1ccccc1C(=O)O] |
| MEOHP | MEOHP-¹³C₄ | 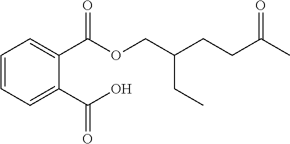 [SMILES: CCC(CCC(C)=O)COC(=O)c1ccccc1C(=O)O] |
| MMP | MMP-¹³C₄ | 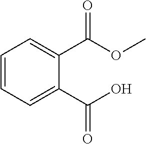 |
| MnOP | MnOP-¹³C₄ | 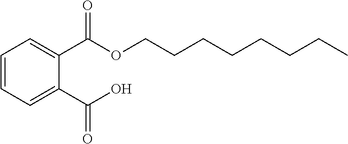 [SMILES: CCCCCCCCOC(=O)c1ccccc1C(=O)O] |
| TPA | TPA-d4 | 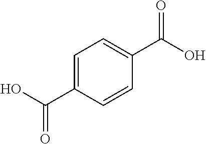 [SMILES: O=C(O)c1ccc(C(=O)O)cc1] |

### III. Sample Processing for Assessing Plasticizer and Plastic Monomers Exposure

Duplicate 100 mL aliquots of raw wastewater sample were acidified to pH 2 using 37% HCl and spiked with a mixture of labeled standards at a concentration of 0.5 ng/mL and extracted using a Dionex™ AutoTrace™ 280 Solid-Phase Extraction (SPE) Instrument (Thermo Scientific, Waltham, MA) with Hydrophilic-Lipophilic Balance (HLB) cartridges (6 cc, 150 mg, 30 μm particle size) manufactured by Waters (Milford, MA). Before extraction, the autotrace was purged with isopropanol to limit potential background contamination of phthalates. SPE cartridges were conditioned with 5 mL of methanol and 5 mL of acidified LC-MS grade water (pH 2) before sample loading. Following sample loading, cartridges were rinsed with 5 mL of pH 2 LC-MS grade water and dried under a stream of nitrogen for 10 min. Target analytes were extracted with methanol containing 2% ammonium hydroxide to a final volume of 1 mL using drip-wise elution of cartridges. Finally, 50 μL of the final extract was dried overnight in a fume hood at ambient temperature and reconstituted in a 20:80 (v/v) methanol and water mixture for analysis. Analyses were performed using triple quadrupole liquid chromatography-tandem mass spectrometry (LC-MS/MS). The enzymatic treatment of wastewater samples was not performed since glucuronide-conjugated phthalate metabolites and bisphenols are quickly hydrolyzed by β-glucuronidase enzymes produced by fecal bacteria present in wastewater.

### IV. Instrumentation for Assessing Plasticizer and Plastic Monomers Exposure

Wastewater extracts were analyzed on a Shimadzu LCMS 8060 with an electrospray ionization (ESI) interface coupled to Shimadzu Nexera™ X2 ultra-HPLC (Shimadzu Scientific, Kyoto, Japan). The chromatographic separation of analytes was completed on a short 5.0×2.1 mm, UHPLC Raptor™ biphenylEXP guard column cartridge (Restek, USA) housed within an EXP® Direct Connect Holder at a flow rate of 0.5 mL/min, similar to a high throughput methodology adopted by (Ng et al., “High-throughput multi-residue quantification of contaminants of emerging concern in wastewaters enabled using direct injection liquid chromatography-tandem mass spectrometry” J. Hazard. Mater., 398 (2020), Article 122933). The binary gradient elution for the analysis of all analytes except for BPA was performed with mobile phases consisting of 0.1% (v/v) acetic acid in LC-MS grade water (A) and 0.1% (v/v) acetic acid in methanol (B). For BPA, mobile phases consisted of ultrapure water (A) and methanol (B). The optimized gradient of 6.5 min for all studied analytes except BPA and BPA-d8 started at 20% mobile phase B for 1 min, followed by a linear increase from 20% to 90% until 3.0 min; held at 90% B for a further 2.0 min, and returned to the initial gradient of 20% B at 5.01 min. The re-equilibration time was 1.5 min. For BPA and BPA-d8, the gradient started at 5% mobile phase B for 1 min, followed by a linear increase from 5% to 65% until 3.0 min; ramped to 100% at 3.01 min, held at 100% B for a further 2.0 min and returned to the initial gradient of 5% B at 5 min. The re-equilibration time was 1.5 min. The injection volume was 5 μl for all analytes except BPA and BPA-d8, which was 10 μl.

High purity argon was used as collision-induced dissociation (CID) gas. Nitrogen and dry air were generated using Proton Onsite DualGas D 251 M LCMS gas generator (Proton Onsite, USA). The ESI was operated in both positive and negative ionization modes. The source parameters were set as follows: Nebulizing gas flow=3 L/min, Heating gas flow=10 L/min, Drying gas flow=10 L/min, Interface temperature=300° C., Desolvation temperature=526° C., Desolvation line temperature=250° C., Heat block temperature=400° C. The MS parameters for target analytes and labeled standards are provided as Table 3.

The raw data were acquired by LabSolutions™ (version 5.93, Shimadzu) and processed using LabSolutions Insight (version 3.2, Shimadzu, Kyoto, Japan). The method development and multiple reaction monitoring (MRM) optimization of each analyte was performed by the flow injection method without an analytical column.

TABLE 3
MS Parameters

| | | | | Retention | | | | |
|---|---|---|---|---|---|---|---|---|
| | | MRM transition | MRM transition | time | Q1 Pre | | Q3 Pre | |
| Analyte | Type | (Quantifier ion) | (Qualifier ion) | (min) | Bias (V) | CE (V) | Bias (V) | ESI |
| BPA | Target | 227.10 > 133.30 | 227.10 > 212.30 | 2.3 | 26, 26 | 24, 17 | 14, 22 | Negative |
| BPA-d8 | ISTD | 235.3 > 137.3 | 235.3 > 220.40 | 2.3 | 22, 27 | 23, 20 | 26, 23 | Negative |
| BPA monosulfate | Target | 307.10 > 227.30 | 307.10 > 212.30 | 2.8 | 20, 36 | 25, 30 | 24, 22 | Negative |
| BPA monosulfate d6 | ISTD | 313.20 > 233.30 | 313.20 > 215.20 | 2.8 | 23, 22 | 24, 32 | 25, 23 | Negative |
| BPAF | Target | 335.10 > 265.20 | 335.10 > 197.30 | 3.0 | 21, 15 | 22, 38 | 28, 38 | Negative |
| BPAF-¹³C₁₂ | ISTD | 347.20 > 277.30 | 347.20 > 208.30 | 3.0 | 28, 16 | 22, 38 | 30, 22 | Negative |
| BPS | Target | 249.20 > 108.20 | 249.20 > 92.10 | 2.3 | 21, 12 | 26, 36 | 21, 17 | Negative |
| BPS-¹³C₁₂ | ISTD | 261.20 > 114.20 | 261.20 > 98.20 | 2.3 | 17, 13 | 28, 36 | 21, 19 | Negative |
| DEHP | Target | 391.00 > 149.30 | 391.00 > 167.30 | 3.5 | −34, −32 | −25, −15 | −30, −34 | Positive |
| DEHP-¹³C₄ | ISTD | 395.10 > 153.30 | 395.10 > 71.40 | 3.5 | −36, −34 | −25, −15 | −30, −12 | Positive |
| DMP | Target | 195.20 > 163.30 | 195.20 > 133.30 | 2.7 | −18, −20 | −11, −23 | −32, −26 | Positive |
| DMP-d4 | ISTD | 199.10 > 81.30 | 199.10 > 166.40 | 2.7 | −21, −16 | −35, −15 | −15, −24 | Positive |
| MBP | Target | 221.20 > 77.10 | 221.20 > 71.10 | 2.9 | 11, 41 | 17, 14 | 30, 28 | Negative |
| MBP-¹³C₄ | ISTD | 225.20 > 79.10 | 225.20 > 180.40 | 2.9 | 26, 26 | 17, 11 | 30, 18 | Negative |
| MBzP | Target | 255.20 > 77.10 | 255.20 > 183.30 | 3.1 | 17, 39 | 25, 10 | 30, 34 | Negative |
| MBzP-¹³C₄ | ISTD | 259.20 > 107.20 | 259.20 > 77.10 | 3.1 | 17, 31 | 15, 25 | 42, 30 | Negative |
| MiBP | Target | 221.20 > 77.10 | 221.20 > 71.20 | 2.9 | 24, 24 | 22, 14 | 30, 28 | Negative |
| MiBP-¹³C₄ | ISTD | 225.20 > 79.10 | 225.20 > 137.20 | 2.9 | 26, 26 | 22, 14 | 32, 26 | Negative |
| MEP | Target | 193.20 > 77.10 | 193.20 > 121.20 | 2.4 | 24, 22 | 15, 15 | 32, 22 | Negative |
| MEP-¹³C₄ | ISTD | 197.20 > 79.10 | 197.20 > 124.20 | 2.4 | 24, 22 | 18, 15 | 32, 22 | Negative |
| MEHP | Target | 277.10 > 127.30 | 277.10 > 77.20 | 3.4 | 30, 18 | 15, 25 | 24, 32 | Negative |
| MEHP-¹³C₄ | ISTD | 281.10 > 79.10 | 281.10 > 137.30 | 3.4 | 18, 24 | 25, 15 | 32, 26 | Negative |
| MEHHP | Target | 291.20 > 121.20 | 291.20 > 143.30 | 3.1 | 34, 20 | 17, 14 | 48, 26 | Negative |
| MEHHP-¹³C₄ | ISTD | 295.30 > 143.30 | 295.30 > 124.20 | 3.1 | 34, 26 | 14, 19 | 26, 22 | Negative |
| MEOHP | Target | 293.20 > 145.30 | 293.20 > 121.20 | 3.0 | 22, 20 | 14, 20 | 26, 22 | Negative |
| MEOHP-¹³C₄ | ISTD | 297.20 > 124.20 | 297.20 > 145.30 | 3.0 | 14, 20 | 19, 14 | 24, 26 | Negative |
| MMP | Target | 179.20 > 77.10 | 179.20 > 107.20 | 2.0 | 21, 16 | 20, 10 | 44, 32 | Negative |
| MMP-¹³C₄ | ISTD | 183.00 > 79.10 | 183.00 > 109.20 | 2.0 | 17, 17 | 20, 10 | 32, 44 | Negative |
| MnOP | Target | 277.20 > 127.30 | 277.20 > 77.10 | 3.4 | 23, 23 | 15, 25 | 50, 31 | Negative |
| MnOP-¹³C₄ | ISTD | 281.20 > 79.10 | 281.20 > 127.30 | 3.4 | 18, 28 | 22, 18 | 32, 24 | Negative |
| TPA | Target | 165.10 > 121.20 | 165.10 > 77.10 | 0.6 | 18, 18 | 15, 20 | 48, 32 | Negative |
| TPA-d4 | ISTD | 169.10 > 125.20 | 169.10 > 81.20 | 0.6 | 16, 17 | 15, 20 | 24, 15 | Negative |

### V. Calculations for the Estimation of Population Normalized Mass Loads and Consumption Assessments

The per capita mass loads and exposure assessments were calculated using equations provided in previous studies. Firstly, the mass load for each compound (mg/day⁻) was calculated by multiplying their respective concentrations (ng/L⁻) in 24-h composite samples with the daily flow rate (L/day⁻) of community wastewater. The daily mass load of each compound was divided by the population assessment of the sewer sub-catchment area to give population normalized mass loads (PNML), expressed in mg/day/⁻1000 people⁻. PNML of each compound is shown in Table 6. The consumption or exposure assessments were calculated by multiplying the PNML with their respective correction factors. The correction factors are the fraction of the molecular weight ratio of the parent compound, the metabolite, and the molar excretion factor. The correction factor of 0.45 was used for calculating BPA consumption assessments using BPA sulfate as the urinary biomarker. The correction factor corresponds to a 3% excretion of BPA sulfate on exposure to BPA.

### VI. Quantification of Analytes and Method Validation

Analytes were quantified using the isotope dilution method, where analyte responses (peak area) were proportional to the responses of corresponding heavy isotope-labeled chemical standards. Each analyte was monitored with two MRM transitions. The most abundant MRM transition was used for quantification, while the second most abundant transition was used for verification. Analytes were identified in wastewater using their retention time and MRM transitions. Reported concentrations were determined based on a minimum 9-point standard curve, with a minimum coefficient of determination of r²=0.99. A 1/x linear regression weighting was used for all analytes to minimize the error due to unequal variance in a wide calibration range, mainly due to the influence of high concentration standards.

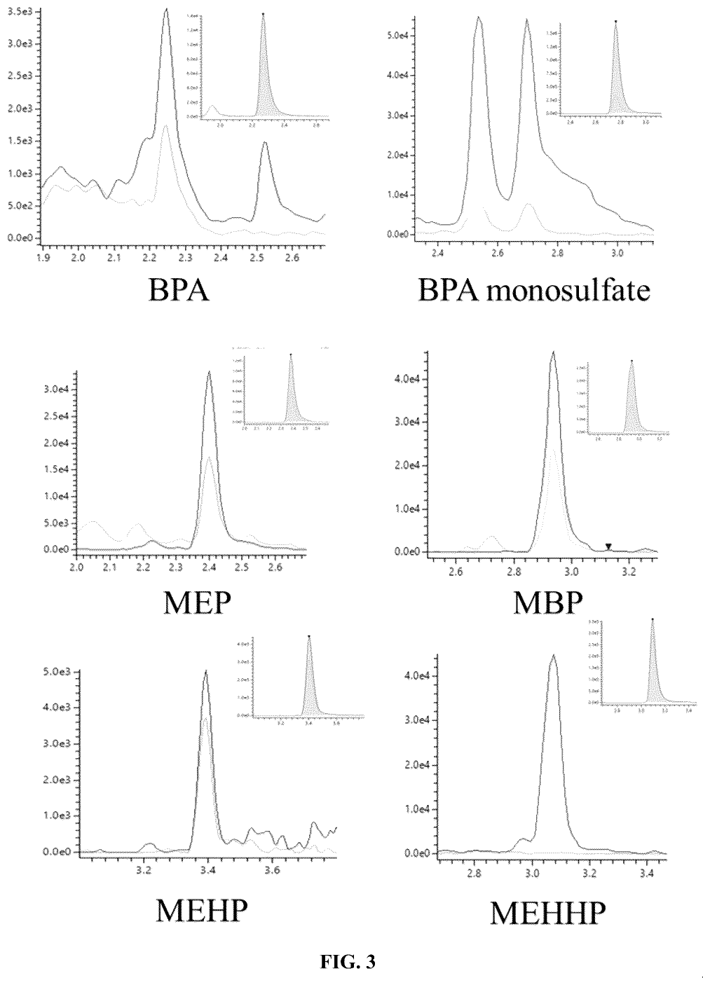

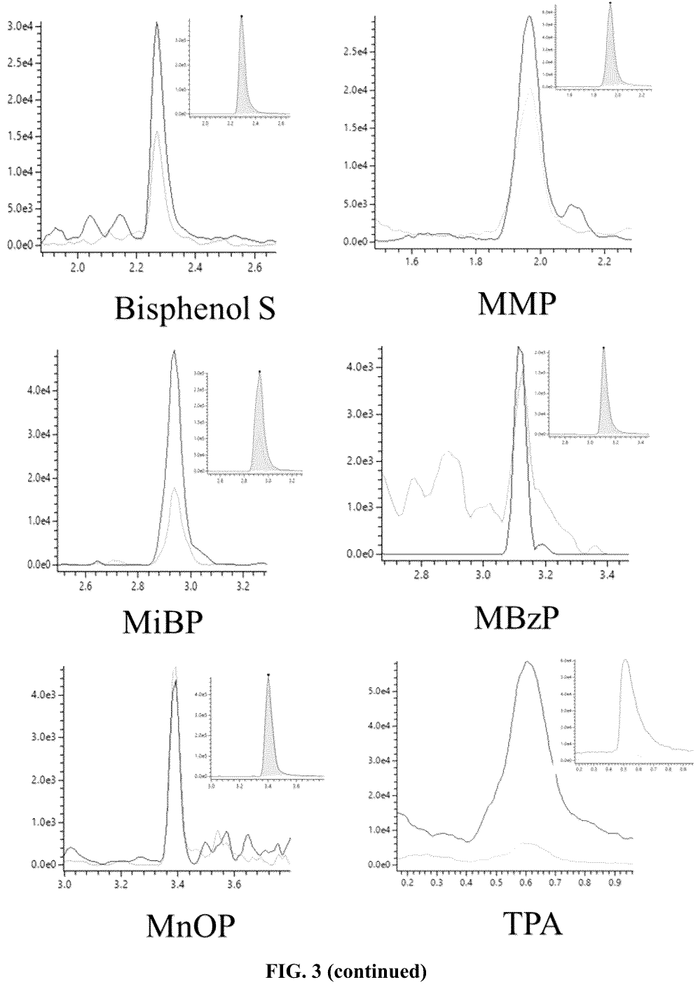

The retention time of analytes in the wastewater matrix was within ±0.2 min of the retention time of the standard compound under the same conditions. One instrument blank was analyzed with each set of 10 wastewater samples. A set of method blanks were processed similarly to samples and analyzed to determine if any background contamination occurred in the analytical procedure. The reported concentrations were calculated by subtracting the average background concentrations from the procedure blanks. The precision of the analysis was measured using relative percentage differences (RPD) between sample duplicates. RPD of duplicates was found within 30% for all samples. The inter-day and intra-day variation in peak areas of all studied analyte standards were <10%. The quantifier to qualifier ion ratio of analytes in target samples was set to be within 30% of the ion ratio of analytes in standards. The chromatograms of analytes detected in raw wastewater are shown in FIG. 3. The method detection limit (MDL) was determined following the EPA guidelines. The analytical MDL ranged from 37 ng/L to 203 ng/L.

Analytes, including DEHP, DMP, MBP, MiBP, MEHP, MMP, and MnOP, were present in blanks. Out of seven analytes detected in procedure blanks, the final concentrations for all except DEHP and DMP were reported by subtracting the background levels in the procedure blank. For DEHP and DMP, it was not feasible to distinguish blank concentrations from the levels present in the wastewater due to high background contaminations. Therefore, this study considered both phthalate parent compounds, DMP and DEHP, non-detect. Stepwise troubleshooting was conducted to identify the primary source of background levels of phthalates. It was hypothesized that the background phthalate levels might be predominantly coming from the LC-MS grade solvents that were used as the mobile phases on the LC-MS/MS; this phenomenon has been reported previously. The presence of phthalate monoesters in blanks has also been reported previously. Phthalate monoesters may be formed due to hydrolysis of phthalate diesters in solvents. The detection of phthalate diesters and monoesters in null injection (without injecting from a vial) indicates that the source of contamination was the mobile phase, the LC flow line, or a combination of the two.

### VII. Concentration of Phthalates, Bisphenols, and Terephthalic Acid in Community Wastewater

Among 16 analytical targets, 10 analytes were detected above the MDL at least once in the community wastewater collected from the southwestern US state. The average concentration of phthalates, bisphenols, terephthalic acid, and their urinary metabolites in raw community wastewater ranged from <MDL to 38 μg/L.

Among bisphenols, bisphenol S was detected with nearly 90% detection frequency with an average concentration of 567±274 ng/L in wastewater. The detection frequency and the average concentration of bisphenol S in this study were higher than in the previous study in the US. BPA was detected only in location A4 at a concentration of 123 ng/L. BPAF was not detected in any sample. A previous study determining levels of bisphenol analogs in the urine of US adults collected between the years 2000 and 2014 also reported the rare detection of BPAF in urine.

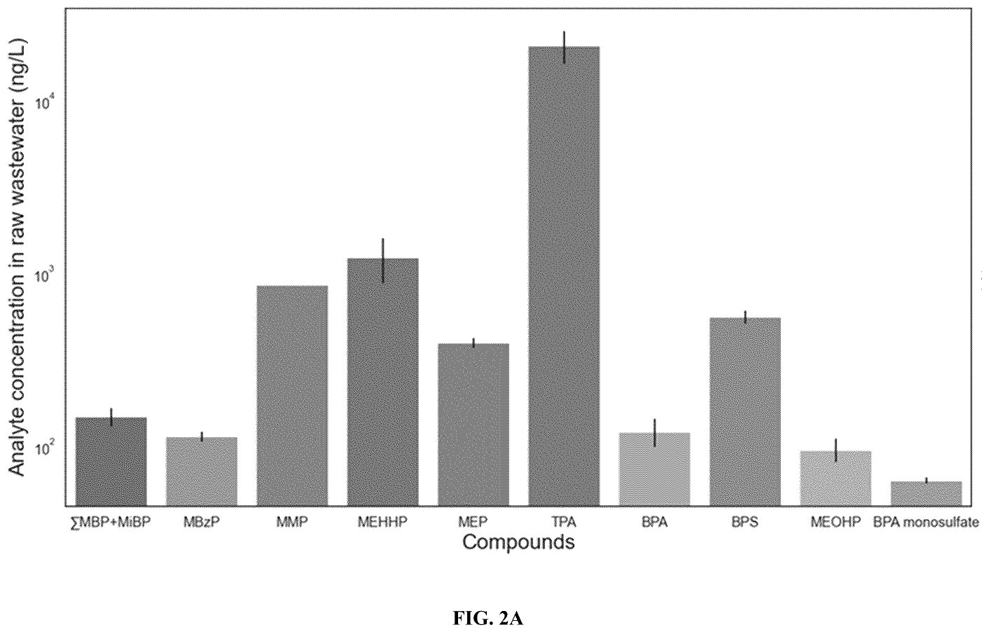

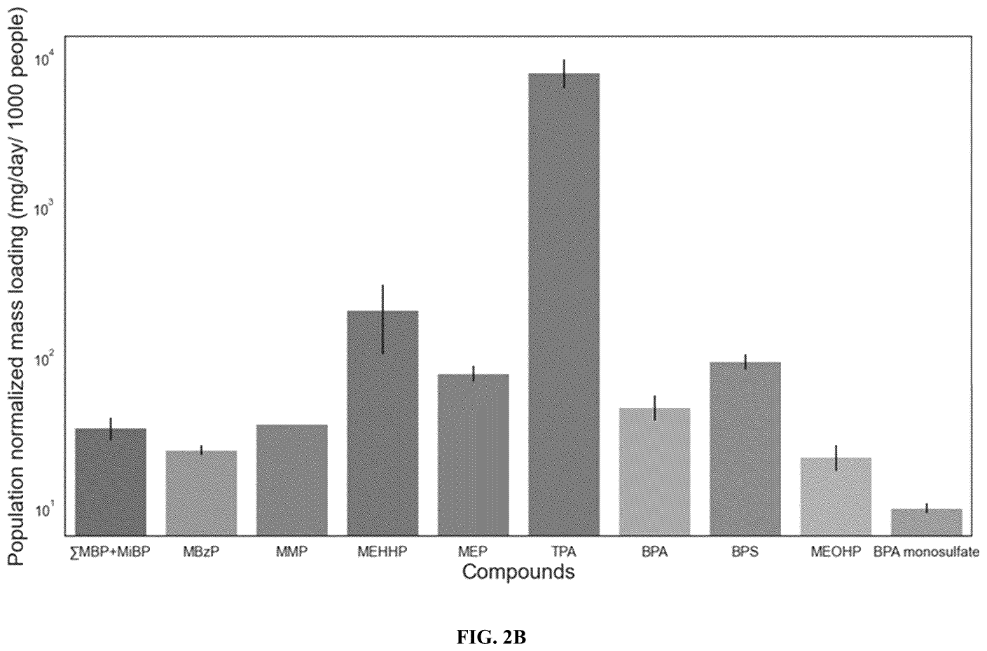

Among phthalate metabolites, monoethyl phthalate (a urinary metabolite of DEP) was detected at the highest frequency with 100% detection at all locations, in agreement with other studies, followed by ΣMBP/MiBP (a urinary metabolite of DBP/DiBP) with almost 70% detection frequency. MEHP, MEHHP, and MEOHP are urinary metabolites of population exposure to DEHP. The wastewater concentration of MEHHP (1250±623 ng/L) was higher than MEOHP (82±21 ng/L) and MEHP (<MDL), similar to the urinary profile of DEHP metabolites. The metabolite of dioctyl phthalate, MnOP, remained undetected in all locations. The average concentration of phthalates, bisphenols, terephthalic acid, and their respective metabolites in community wastewater, along with their frequency of detection and MDLs, are shown in Table 5 and FIG. 2A. The concentration of analytes in individual sewersheds is shown in Table 6.

TABLE 5
Occurrence of analytes in community wastewater
(n = 35) along with their respective MDLs.

| | | Average | | | |
|---|---|---|---|---|---|
| | | measured | | Detection | |
| | | concentration | Range | frequency | MDL |
| Analyte | Parent compound | (ng/L) | (ng/L) | (%) (n = 35) | (ng/L) |
| BPA | Polycarbonate | 123 ± 33 | N.D.-146 | 6 | 58 |
| BPA | BPA | 64 ± 6 | N.D.-74 | 17 | 59 |
| monosulfate | | | | | |
| Bisphenol S | Bisphenol S | 567 ± 274 | N.D.-976 | 89 | 79 |
| MBzP | Butylbenzyl | 116 ± 19 | N.D.-138 | 17 | 90 |
| | phthalate | | | | |
| MEHHP | di (2-ethylhexyl | 1250 ± 623 | N.D.-1952 | 9 | 203 |
| | phthalate) (DEHP) | | | | |
| MEHP | DEHP | <MDL | N.D. | 0 | 63 |
| MEP | Diethyl phthalate | 401 ± 149 | 100-820 | 100 | 88 |
| MMP | Dimethyl phthalate | 868 | N.D.-868 | 3 | 119 |
| MnOP | Dioctyl phthalate | <MDL | N.D. | 0 | 84 |
| TPA | Polyethylene | 20,951 ± 10,179 | N.D.-38,653 | 14 | 37 |
| | terephthalate | | | | |
| ΣMBP, | Dibutyl | 150 ± 52 | N.D.-338 | 71 | 51 |
| MiBP | phthalate/Diisobutyl | | | | |
| | phthate | | | | |
| MEOHP | DEHP | 82 ± 21 | N.D.-179 | 11 | 60 |

N.D. means non-detect

TABLE 6
Concentration (ng/L) of analytes detected in community wastewater collected
from 5 sewer sheds of the city in the Southwestern US state.

| | Locations | | | | |
|---|---|---|---|---|---|
| Analytes | A1 | A2 | A3 | A5 | A5 |
| Bisphenol A | N.D. | N.D. | N.D. | 123 ± 33 | N.D. |
| Bisphenol A monosulfate | <MDL | 60 | 65 ± 7 | N.D. | <MDL |
| Bisphenol S | 722 ± 122 | 656 ± 321 | 696 ± 114 | 102 ± 16 | 542 ± 125 |
| Monobenzyl phthalate | <MDL | 111 ± 17 | 138 | <MDL | <MDL |
| MEHHP | 1952 | 760 | N.D. | <MDL | 1039 |
| MEHP | N.D. | N.D. | N.D. | N.D. | N.D. |
| Monoethyl phthalate | 331 ± 28 | 547 ± 73 | 306 ± 162 | 376 ± 203 | 447 ± 90 |
| Monomethyl phthalate | N.D. | <MDL | N.D. | <MDL | 868 |
| Monooctyl phthalate | N.D. | N.D. | N.D. | <MDL | N.D. |
| Terephthalic acid | N.D. | N.D. | N.D. | 21,000 ± 10,000 | N.D. |
| ΣMBP, MiBP | 90 ± 36 | 271 ± 92 | 93 | 133 ± 76 | 101 ± 31 |
| MEOHP | 72 | 1091 ± 21 | <MDL | <MDL | <MDL |

ND. Non detected;
MDL Method Detection limit (Refer to Table 1 for MDL values)

### VIII. Estimation of PNML and Communities' Exposure to Plasticizers

PNML of plastic monomers and plasticizers in community wastewater were calculated as described. The monomer of PET and TPA were detected only in location A4 at the average PNML of 7860±3820 mg/d/1000 people. The frequent detection of TPA in A4 and non-detects at other locations indicates that people working in A4 are potentially more exposed to TPA than at other sites. Location A4 has primarily commercial activities with a small population of people working in offices. PET bottles are used as packaging material for bottled water, beverages, drinks, canned foods, and emissions from carpets. The occurrence of TPA in the environment may be due to PET degradation in the environment and leaching from PET bottles. TPA is also used for manufacturing polyester fibers and films. Also, it is worth noting that TPA is not metabolized in the human body and is excreted unchanged in the urine.

BPA is metabolized to BPA glucuronide upon human uptake and eliminated through urine. However, sulfated-, hydroxylated-, and carboxylated-BPA are released in human urine as minor metabolites. BPA sulfate was analyzed in this study instead of free BPA to assess community exposure to BPA because BPA sulfate is the unique urinary biomarker of BPA exposure in humans. BPA sulfate has already been utilized in WBE studies as a biomarker for community-wide exposure to BPA. BPA sulfate has been reported as a highly stable biomarker suitable for WBE applications. In contrast, there may be several sources of free BPA in wastewater apart from urinary excretion, including industrial discharge, leaching from packaging plastics and food cans, medical products, and dental sealant. Therefore, BPA sulfate can be considered a more suitable biomarker than free BPA for assessing BPA exposure. BPA sulfate, an indicator of human exposure to BPA, was detected only in predominantly residential locations, including A2 and A3. A5, being primarily commercial sites with a small catchment population (Table 1), had BPA sulfate levels below MDL. BPA sulfate was not detected in location A4. The estimated average exposure to BPA by communities in A2 and A3, measured using its urinary metabolite, BPA sulfate, were 6 mg/d/1000 people and 4 mg/d/1000 people, respectively. The average consumption assessments of BPA in this study were significantly lower than the study done in the UK.

In the case of BPS, BPS sulfate could have been the more accurate and unique biomarker of human exposure to BPS. However, due to the unavailability of the isotope-labeled standard, BPS sulfate was not included in the study. Moreover, there is no data in the literature regarding the stability of BPS sulfate in wastewater. As a result, only mass loadings of BPS were calculated by analyzing free BPS in community wastewater. The average mass loading of BPS in the current study (91±59 mg/d/1000 people) was found to be higher than the previous study done in New York (19 mg/d/1000 people). BPS is a widely used BPA alternative and is not regulated in several countries, including the US. Although initially considered safe, there is a lack of scientific consensus on the risk imposed by BPS. BPS is used as the monomer to manufacture plastics and epoxy in plastic materials of daily use, dental materials, personal care products, and paper products. Dietary sources are the primary source of BPS exposure in the population. The frequent detection of BPS in all sampling locations indicates the ubiquitous use of BPS in plastic products. However, it Is essential to note that the residential sub-catchments with a large population (A1, A2, and A3) had higher mass loadings of BPS, indicating higher exposure than the predominantly commercial sub-catchments with a small population (A4 and A5). In A4, the BPS and BPA concentrations were almost comparable (BPS/BPA˜0.8), indicating a gradual increase in BPS use in consumer products.

A recent study reported rapid transformations of phthalate diesters to phthalate monoesters and degradation of phthalate metabolites in laboratory sewer reactors and suggested that these metabolites may not be appropriate for WBE applications. However, other studies successfully employed the WBE approach to assess phthalate exposure to communities. Nevertheless, the consumption or exposure of most phthalates in communities were not assessed due to the lack of scientific consensus on the suitability of phthalate monoester biomarkers as WBE biomarkers.

The PNML of DEHP urinary metabolite, MEHHP was estimated as 391 mg/d/1000 people in the location A1, 181 mg/d/1000 people in the location A2, and 47 mg/d/1000 people in the location A5. MEHHP was not detected in locations A3 and A4. MEHHP average mass loading in this study was found to be higher than the mass loadings reported in Australian wastewater (<100 mg/d/1000 people). The parent DEHP is rarely detected in urine and is not considered a suitable biomarker. In this study, MnOP (a urinary metabolite of dioctyl phthalate) was undetected, agreeing with the National Health and Nutrition Examination Survey (NHANES) survey. NHANES surveys in 1999-2000 and 2005-2006 indicated infrequent or no detection of urinary MnOP.

The metabolite of the low molecular weight phthalate (DEP, for example) was the most frequently detected in wastewater compared to high molecular weight phthalates (DEHP, BBzP). MEP is the major metabolite of DEP metabolism in humans. In agreement with clinical studies reporting very high detection frequencies of MEP in human urine, MEP was detected at all locations in this study. This indicates widespread exposure of communities to DEP. The average PNML of MEP was 79±53 mg/d/1000 people. MEP mass loading in this study was found to be much lower than the average loading reported in Australian wastewater (>400 mg/d/1000 people). DEP is mostly used in cosmetics and personal care products, in addition to its uses as plasticizers, toothbrushes, and automotive parts.

The urinary metabolite of DMP exposure, MMP, was detected only once above the MDL at the PNML of 36 mg/d/1000 people. DMP is used in consumer products, including plastics and insect repellents. In addition to consumer products, it is also used to manufacture solid rocket propellent. MMP has been reported sparingly in urine samples of the US population and most the countries around the globe, consistent with the findings of this study in community wastewater.

Σ(MBP, MiBP) are the metabolites of DBP and DiBP exposure to humans. DBP/DiBP are mostly used in personal care products, including cosmetic products, pharmaceutical coatings, insecticides fabrics, polyvinyl floorings, and products. Σ(MBP, MiBP) have been detected in all five locations at an average PNML of 28±24 mg/d/1000 people.

Monobenzyl phthalate, a urinary metabolite of exposure to benzylbutyl phthalate, was detected only in two locations above the MDL at an average PNML of 24 mg/d/1000 people, in a similar range as Australian wastewater. Benzylbutyl phthalate is primarily used in adhesives, automotive care products, sealants, vinyl tiles, and personal care products. However, diet is considered the major source of community exposure.

The average PNML of phthalates, bisphenols, terephthalic acid, and their respective metabolites in community wastewater is shown in FIG. 2B. The PNML of analytes in individual sewersheds is shown in Table 4.

TABLE 4
Population normalized mass loads (mg/d/1,000 people) of
analytes detected in community wastewater collected from
5 sewer sheds of the city in the Southwestern US state.

| | Locations | | | | |
|---|---|---|---|---|---|
| Analytes | A1 | A2 | A3 | A5 | A5 |
| Bisphenol A | N.D. | N.D. | N.D. | 47 ± 13 | N.D. |
| Bisphenol A monosulfate | <MDL | 13 | 9 ± 1 | N.D. | <MDL |
| Bisphenol S | 142 ± 23 | 150 ± 71 | 100 ± 16 | 38 ± 6 | 20 ± 6 |
| Monobenzyl phthalate | <MDL | 25 ± 4 | 20 | <MDL | <MDL |
| MEHHP | 393 | 181 | ND | <MDL | 47 |
| MEHP | N.D. | N.D. | N.D. | N.D. | N.D. |
| Monoethyl phthalate | 65 ± 5 | 126 ± 14 | 44 ± 24 | 141 ± 77 | 19 ± 5 |
| Monomethyl phthalate | N.D. | <MDL | N.D. | <MDL | 36 |
| Monooctyl phthalate | N.D. | N.D. | N.D. | <MDL | N.D. |
| Terephthalic acid | N.D. | N.D. | N.D. | 7,967 ± 3,941 | N.D. |
| ΣMBP, MiBP | 18 ± 8 | 58 ± 22 | 14 | 50 ± 29 | 4 ± 1 |
| MEOHP | 14 | 26 ± 4 | <MDL | <MDL | <MDL |

### IX. Systematic Scoping of Literature Review for Determining Wastewater Biomarkers Indicating the Prevalence CVD and Caner

A systematic scoping review of the literature was performed following published guidelines. Accordingly, literature inclusion and exclusion criteria were established first to inform the scanning and selection of literature appropriate for critically examining the research question of whether WBE has known or potential utility for studying CVD and cancer at the population level.

An initial search of the literature was performed using the SCOPUS database for recently reported publications (within 5 years from May 2022). For case-study relevant background information, secondary searches were limited to the United States. To search for literature that reported on cardiovascular disease (CVD) and individual diagnostic biomarkers, the search terms included “cardiovascular disease” OR “CVD” OR “heart disease” AND “biomarkers” OR “diagnostic” OR “endogenous” OR “human”. Search terms for cancer and individual diagnostic biomarkers included: “cancer” OR “breast cancer” OR “liver cancer” OR “prostate cancer” AND “biomarkers” OR “diagnostic” OR “endogenous”.

### a. Inclusion and Exclusion Criteria

Studies were excluded if there was no report of urinary or fecal biomarkers, if animal models were used as proxy to humans, and if there were no associations found between the biomarkers under investigation and the diseases of interest. From these searches, papers were then cross-examined to identify studies that reported hazard ratios (HR), or an equivalent metric, to evaluate associations and specificity between identified biomarkers and either CVD or cancer, and relevant disease subsets, and if there were significant differences in urinary concentrations between patients and controls. Studies that reported hazard ratios as null (HR=1) were excluded, while studies reporting low (HR<1) or strong (HR>1) association to either CVD or cancer were included for further evaluation. Studies were also excluded if only creatinine-normalized values were reported for urinary concentration of select biomarkers, or if studies only reported detections in a matrix less likely to consistently and reliably contribute to community wastewater (i.e., blood, sputum).

### b. Systematic Scoping Literature Analysis Results

A total of 48 peer-reviewed papers were critically examined as a result of this analysis that offered insightful elements on disease etiology, epidemiological information, biochemical pathways and reactions, biomarker discovery, and clinical investigations. All studies identified biomarker detections in either blood (serum or plasma) or urine; no studies indicated fecal excretion as a dominant or viable excretion route. From those reported detections, five urinary protein biomarkers were reported as indicative of specific subsets of CVD and cancer: heart attack/heart failure, atherosclerosis, cancer of the liver, prostate, and breast, and tumor presence (Table 7).

TABLE 7
Identified endogenous biomarkers proposed for population-
level detection of CVD and cancer by WBE.

| | Molecular | Detectable | Disease |
|---|---|---|---|
| Biomarker | Weight (kDa) | Matrix | Indicator |
| Cardiac- | 24 | Blood, Urine | Myocardial |
| Troponin I | | | infarction |
| (cTnI) | | | (heart attack), |
| | | | heart failure |
| Cystatin C | 13 | Blood, Urine | Atherosclerosis |
| α-fetoprotein | 70 | Blood, Urine | Prostate & liver cancer |
| NMN | <1 | Blood, Urine | Tumor presence |
| | | | (pheochromocytoma) |
| MAST4* | 260 | Blood, Urine | Breast Cancer (DCIS) |

MAST4: Microtubule associated serine/threonine kinase 4;
NMN: Normetanephrine;
DCIS: Ductal carcinoma in-situ
*Calculated in this study

### X. Calculated Ratios and Population-Level Biomarker Concentrations

Ratios were calculated for each biomarker to evaluate the degree of difference between diseased and healthy individuals using literature reported urinary concentrations (ng/mL) following Equation 1:

Disease:Healthy Ratio=D/H 1

Where D is the literature reported urinary concentration (ng/mL) that indicates disease and H is the literature reported urinary concentration (ng/mL) of healthy individuals based on clinical investigations. This calculation was repeated for each biomarker of interest for both CVD and cancer and affiliated subset morbidities.

Estimated population-level biomarker concentration thresholds (ng/L/10,000 population) that would indicate disease presence was calculated using literature reported values identified in human urine using Equation 2:

$$\frac{IE \times HU \times pop}{F} \times P R \qquad Eq . 2$$

Where IE is the literature reported biomarker value (ng/mL) in human urine at the individual level. HU is the average number of liters an individual excretes urine per day (1.4 L). Pop and F represent theoretical values for population and flow rate utilized for all calculations in this exercise. Here, a population size of 10,000 contributing individuals with a 0.9 million gallons per day (MGD) flow rate of wastewater were assumed to accomplish this exercise. PR is the most recently reported estimated prevalence of CVD (7.2%) (CDC, 2022a) and cancer (5.1%) (NCI, 2020a) in the United States. Conversion from MGD to L/day was used following Equation 3:

L/day=flow (MGD)*M*CF Eq. 3

Where flow (MGD) is the reported flow rate at time of sample collection (commonly as million gallons per day) multiplied by M (1,000,000) to convert from million gallons per day to gallons per day. CF is the conversion factor from gallons to liters (3.785).

### XI. Cardiovascular Disease and Cancer Burden in the United States

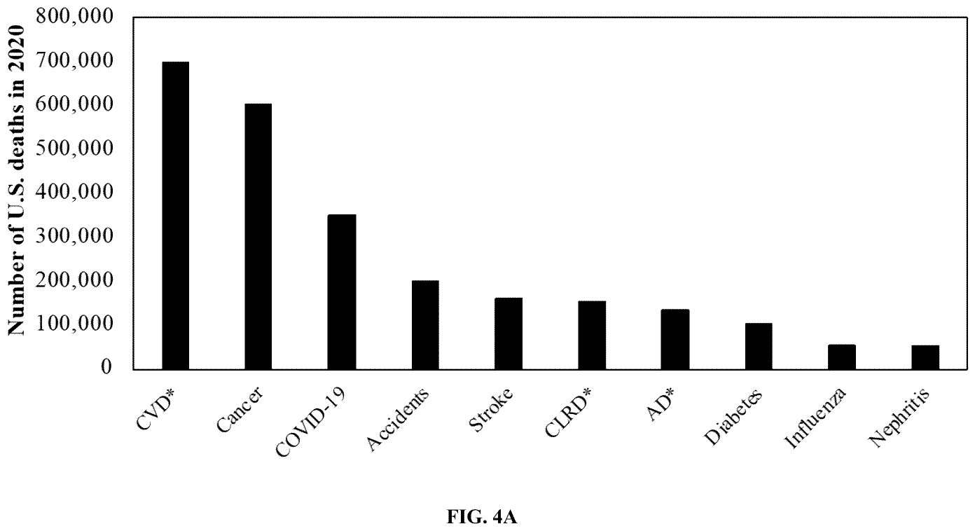

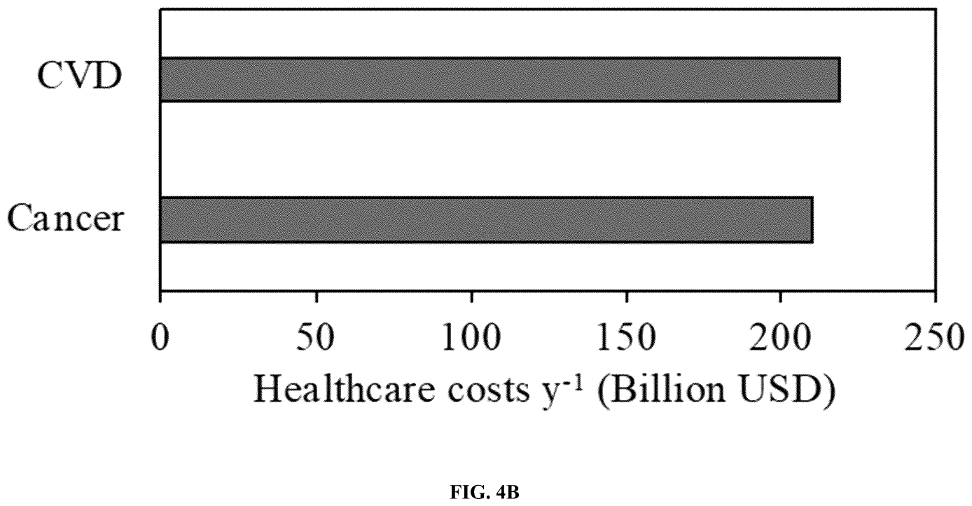

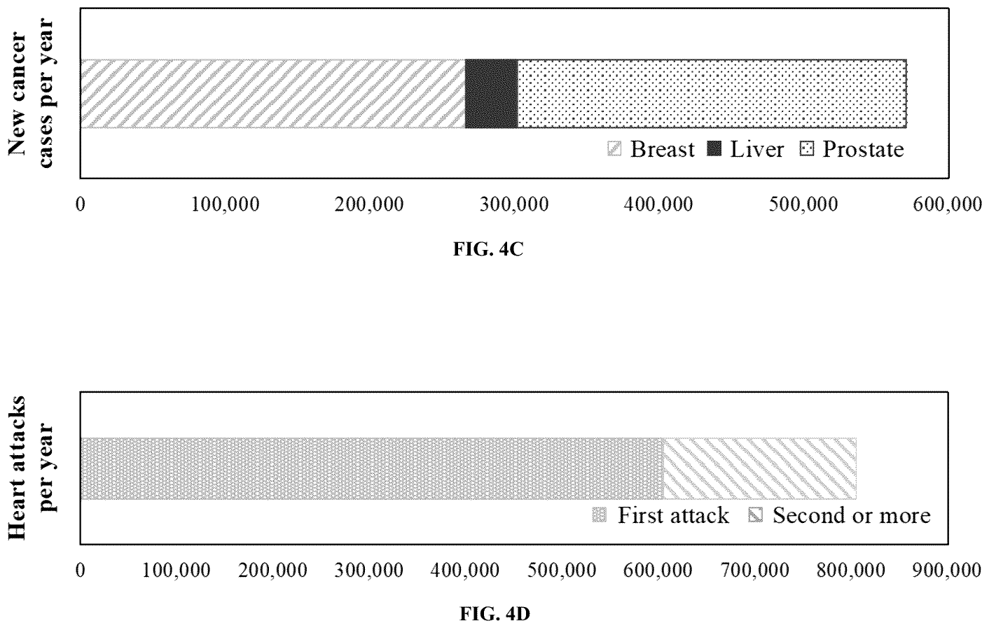

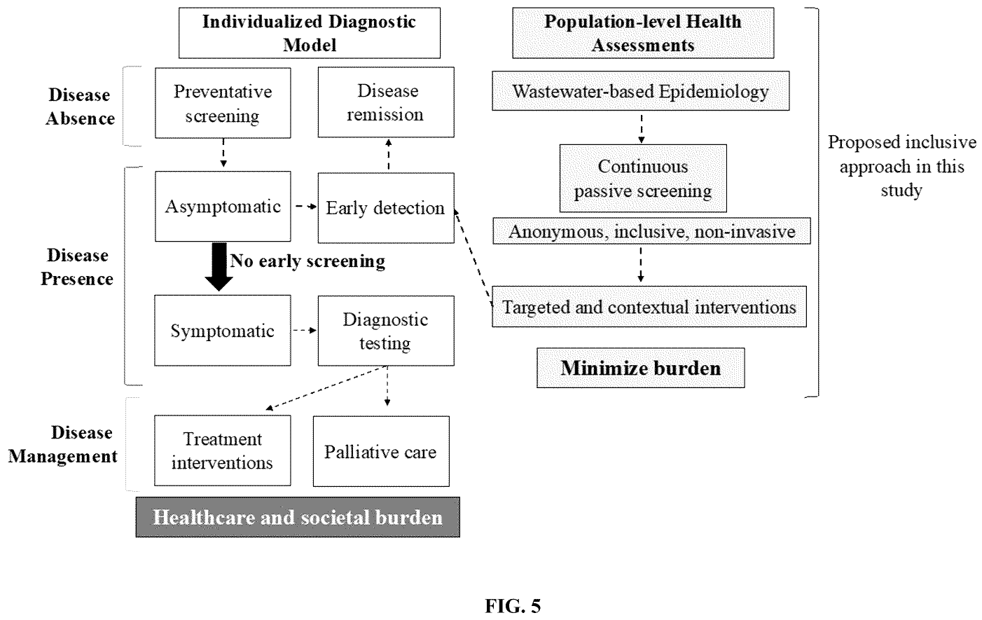

CVD has consistently been the leading cause of death in the United States since 1910. CVD is a broad term used to refer to several sub-classifications of cardiovascular-related conditions such as peripheral arterial disease, congestive heart failure, myocardial infarction (heart attack), arrhythmias, and stroke. As of 2016, an estimated 121 million people in the United States, 48% of the total population, are suffering from some form of CVD-related morbidity, with atherosclerosis (hardening of blood vessels due to plaque buildup) being the leading underlying cause of heart attacks affecting nearly 805,000 people per year (FIGS. 4A-4D). Risk factors for the development of CVD are vast and can include tobacco use, poor dietary and/or exercise behavior, high alcohol consumption, family history of heart disease, age, sex, ethnicity, and toxic environmental exposures. If an individual is already at risk, such as having a family history of the disease, it greatly increases their chance of developing CVD throughout their lifetime, for example, individuals who have a parent with CVD have up to a 75% increased risk of developing CVD compared to those who do not have a family history. This raises the importance of adopting healthier lifestyle behaviors for those individuals, such as diet and exercise, as well as adopting a routine of preventative screening in order to thwart a sudden and potentially fatal cardiac event.

The conventional approach in a clinical environment for diagnosing CVD is performed primarily through a comprehensive panel of diagnostic bloodwork, however, new advances in diagnostic medicine have determined less-invasive urinary measurements are also feasible for certain biomarkers at the individual level. Particular biomarkers of interest for assessing various cardiovascular-related conditions include those that indicate acute myocardial infarction (heart attack) and atherosclerosis due to their widespread prevalence and correlations to CVD-related deaths. Atherosclerosis is an inflammatory disease that can lead to the hardening of blood vessels through the accumulation of plaque; increasing the risk for heart attack, abnormal heart rhythm (arrhythmias), stroke, as well as increasing the risk for other life-threatening co-morbidities such as diabetes or kidney disease. Several of these conditions can quickly become fatal if this buildup of vascular plaque is not detected early, highlighting the importance in support of regular screening methods.

Cancer has remained the second leading cause of death in the United States since 2016, with an estimated 599,108 lives lost per year. As of January 2019, approximately 16.8 million Americans are living with cancer, and an additional 1.9 million new cases are estimated to be diagnosed in 2022. Cancer is generally defined as an abnormal, unrestricted growth of cells able to occur anywhere in the body and in any organ or structure at any given time. A principal trait of cancerous cells is their ability to proliferate chronically and exponentially, whereas normal cells control their numbers through mechanistic growth signals; ensuring homeostasis throughout the body and proper functionality. Some of the most common types of cancer in the United States include cancer of the breast, prostate, and liver (FIG. 4C).

Cancer is commonly referred to as a silent disease as many of the more noticeable symptoms do not arise until the later stages in progression, in some cases when it's too late for treatment, or the symptoms are non-specific that could potentially be explained by another less severe condition. Evidence suggests the risk of developing cancer is much greater in individuals who have weakened immune systems due to age, underlying conditions, or chronic stress, with socioeconomic status serving as a major driving factor for incidence as well as diminished survival rate due to late diagnosis). These circumstances thus further complicate an already dangerous and complex disease and can make it nearly impossible to effectively and time-efficiently diagnose and treat an individual thought to have cancer. Thus, similar to CVD and due to the particularly complex nature of cancer, continuous screening events that allow for early detection of disease play an essential role in mitigating further progression and effectively treating many types of cancer, such as of the liver, prostate, and breast, and in some cases, can be cured if detected early enough.

As such, more initiatives are advocating for the adoption of novel early detection and targeted diagnostic techniques for cancer and CVD, with an emphasis on less invasive sample collection matrices, (i.e., urine), and integrating a “systems biology” approach involving multiomic analyses such as metabolomics, genomics, and proteomics; the latter at the heart of most of these investigations. Potentially feasible, but still in its infancy, biomarkers identified in individualized diagnostic research could also be monitored at population-scale to inform on the threat potential and health status of entire populations, rather than merely individuals. This type of approach could offer to inform more contextually relevant strategies and interventions to be applied that are near-pace with incidence rates of these deadly diseases.

### XII. Identified Biomarkers and Association Analysis to Cardiovascular Disease and Cancer

As mentioned, diagnosing CVD and cancer can become a time-consuming and expensive endeavor, however, recent clinical advances that have identified biomarkers excreted in urine hold great promise for proposing investigation via WBE. These biomarkers for population-level detection and assessment for CVD include high-sensitivity cardiac troponin I (cTnI), indicative of myocardial infarction (heart attack) and heart failure, and cystatin C, which has been reported to potentially serve as an indicator for atherosclerosis (Table 7). A common metric to measure strength of association between an exposure and/or event (i.e., a measured biomarker) and a disease under investigation in prospective controlled clinical studies is the hazard ratio (HR) reported on a scale as either less than, equal to, or greater than 1 with 95% confidence intervals (CI); low association (HR<1), lack of association (HR=1), and strong association (HR>1) (Toledo, 2018). A study investigating the strength of using cTnI as an indicator of an upcoming cardiovascular event, such as a heart attack, in 1,177 individuals exhibited a hazard ratio (HR) of 1.24 (95% CI, 1.17-1.32), suggesting it to be a strong candidate to serve as an early warning signal for a potentially fatal event. Another study reported that first-morning urinary cTnI concentrations greater than 4.1×10⁻³ ng/mL were associated with an increased risk of an upcoming (short-term) cardiovascular event compared to controls, reported as an odds ratio (OR), a similar metric to the hazard ratio, of 3.043, 95% CI 1.448-6.391 (p=0.003).

Atherosclerosis is an inflammatory disease that can lead to the hardening of blood vessels through the accumulation of plaque. In western societies, such as the United States, atherosclerosis is the primary cause of heart disease and stroke and is responsible for approximately 50% of all deaths. In a prospective study of 1,004 patients investigating CVD in patients with and without atherosclerosis, cystatin C was significantly (p<0.001) associated with predicting a future cardiovascular event in those with atherosclerotic plaque or those who developed plaque over time; HR 1.94 (95% CI, 1.31 to 2.88). Human clinical urinary threshold concentrations for cTnI and cystatin C that would suggest an upcoming or current occurrence of a cardiovascular event or buildup of plaque at the individual-level have been reported as 3.9×10⁻² ng/mL and 2.8×10³ ng/mL, respectively, compared to reported typical urinary concentration ranges in healthy individuals of 6.0×10⁻³-3.0×10⁻³ ng/mL for cTnI and 5.2×10¹-1.4×10² ng/mL for cystatin C (Table 8).

TABLE 8
Reported healthy reference ranges in urine along with clinical
urinary threshold values of proposed biomarkers indicative
of presence of individual disease related to CVD and/or cancer
in individuals. Calculated disease to healthy ratios used
to determine fold-changes between literature reported values
of healthy controls versus patients with disease.

| | Healthy | Reported Urinary | |
|---|---|---|---|
| | Reference | Disease | Disease to |
| | Ranges | Thresholds | Healthy |
| Biomarker | (ng/mL) | (ng/mL) | Ratio* |
| Cardiac- | 0.01-0.03 | 0.04 | 2.0 ± 1.3 |
| Troponin I | | | |
| (cTnI) | | | |
| Cystatin C | 52-140 | 280 | 2.9 ± 2.6 |
| α-fetoprotein | ND-0.01 | 0.03 | 2.6 ± 2.0 |
| NMN | 34-530 | 740 | 6.0 ± 3.2 |
| MAST4 | N/A | N/A | 4.2** |

MAST4: Microtubule associated serine/threonine kinase 4;
NMN: Normetanephrine;
ND: Non-detect;
N/A: Not applicable
*Calculated in this study
**MAST4 was found to be upregulated by 4.2-fold in breast cancer patients compared to healthy controls using relative quantification label-free LC-MS/MS

Cancer is another highly complex and prevalent disease that could be mitigated through early detection and screening efforts, particularly at population-scale. Recent studies have identified a few promising biomarkers for this purpose, including α-fetoprotein, predominately for the detection of prostate and liver cancer, normetanephrine for identifying tumors such as pheochromocytoma, and microtubule assisted serine/threonine kinase 4 (MAST4), for the detection of breast cancer (Table 7). Breast cancer is the most common type of cancer in women in the United States, constituting about 30% of all new female cancer cases per year. One study that compared the urinary proteome using a semi-quantitative, label-free LC-MS/MS approach between patients with breast cancer and healthy women controls found that MAST4 was up-regulated by 4.2-fold in the breast cancer patients, indicating it could be used as a strong biomarker for regular screening at the individual-level. Hepatocellular carcinoma (HCC) is one of the most invasive types of cancer in humans and remains to be the third leading cause of death due to cancer across the globe, while prostate cancer affects approximately 1 in 8 men throughout their lifetime. The protein α-fetoprotein is commonly used as a clinical biomarker for the detection, prognosis, and management of both prostate and liver cancer, specifically HCC, most typically through serum detection followed up with ultrasound confirmation, however, recent studies have found elevated urinary levels exhibited in hepatocellular carcinoma patients compared to controls (HR 1.55; 95% CI 1.3 to 1.8).

Normetanephrine is produced by the action of a catechol-O-methyltransferase enzyme from norepinephrine that is excreted in urine and is commonly used as a marker for dangerous tumors such as pheochromocytoma; a neuroendocrine tumor that can lead to detrimental comorbidities including hypertension, chronic headaches, and damage to various organs such as the liver and adrenal glands. While pheochromocytomas are often considered rare, it is estimated that current prevalence rates are vastly underreported due to their nonspecific presentation of symptoms. Nonetheless, normetanephrine has been indicated to be a strong indicator for pheochromocytoma, with increases measured in plasma as high as 36-fold when compared to healthy controls. Studies reporting urinary thresholds that can indicate potential presence of cancer at the individual level are 2.6×10⁻² ng/mL and 7.4×10² ng/mL for α-fetoprotein and normetanephrine, respectively, compared to healthy urinary concentration ranges reported as non-detect (ND) to 1.0×10⁻² ng/mL for α-fetoprotein, and 3.4×10¹-5.3×10² ng/mL for normetanephrine (Table 8). These literature-reported urinary concentrations can be used as references when predicting and/or comparing to data derived from wastewater. Further, ratios of biomarker levels in diseased and healthy individuals were calculated based on literature reported urinary concentrations (ng/mL) in order to potentially differentiate presence and absence of disease based on clinical evaluations. Results from these calculations were as follows: 2.0±1.3 for cTnI, 2.6±2.0 for α-fetoprotein, 2.9±2.6 for cystatin C, and 6.0±3.2 for normetanephrine. The protein biomarker indicative of breast cancer, MAST4, was reported to be upregulated by 4.2-fold in patients compared to healthy controls using label-free relative quantification by LC-MS/MS (Table 8).

### a. Estimated Population-Level Biomarker Concentrations in Wastewater

While the ratios are informative to provide insight on the difference in up-regulation in diseased individuals, it was necessary to translate these literature-reported urinary concentrations for each biomarker into a theoretical exercise based on real-world values for detection in community wastewater. Calculations were performed assuming the scenario of investigating within a relatively small wastewater catchment at neighborhood-scale comprised of a population of 10,000 contributing individuals resulting in approximately 0.9 million gallons per day (MGD) of wastewater flow. This analysis revealed that all identified biomarkers, with the exception of MAST4 which has only been reported thus far as a presence/absence biomarker, resulted in concentrations that could theoretically be monitored in wastewater based on these assumptions (ng/L/1,000 population): cTnI (0.02), cystatin C (115), α-fetoprotein (0.01), and normetanephrine (689), with cystatin C and normetanephrine suggesting to be the most promising biomarkers given their elevated concentrations in comparison to the others, which is in alignment with the previous disease to healthy ratio exercise (Table 9). Next, prevalence rates for CVD (7.2%) and cancer (5.1%) were incorporated to adjust for disease prevalence within this simulated population size of 10,000 individuals as it is relevant to the United States (ng/L): cTnI (0.01), cystatin C (83), α-fetoprotein (0.01), and normetanephrine (351) (Table 9). Based on these prevalence-adjusted population-level concentrations, the strongest biomarkers that present with the most promise for a WBE study are normetanephrine and cystatin C, followed by cTnI and α-fetoprotein. Normetanephrine and cystatin C represent both CVD and cancer-related morbidities, such as tumor presence and atherosclerosis, thus, incorporating WBE may serve to enhance current public health monitoring efforts.

TABLE 9
Calculated population-level urinary thresholds of proposed biomarkers
determined by WBE in a population of 10,000 with an average flow
rate of 0.9 million gallons per day (MGD). All concentrations
reported represent (left to right) the entire population (10,000),
a standardized portion of the population (per 1,000), and prevalence-
adjusted based on reported values for the United States. Note that
MAST4 was not included in this analysis.

| | Calculated Disease Concentrations in Wastewater | | |
|---|---|---|---|
| | (ng/L/ | (ng/L/ | Prevalence-adjusted* |
| Biomarker | 10,000) | 1,000) | (ng/L) |
| Cardiac-Troponin I | 0.20 | 0.02 | 0.01 |
| (cTnI) | | | |
| Cystatin C | 1,200 | 115 | 83 |
| α-fetoprotein | 0.10 | 0.01 | 0.01 |
| Normetanephrine | 6,900 | 689 | 351 |
| (NMN) | | | |

*Prevalence rates in the U.S.: CVD 7.2%; Cancer 5.1%

### XIII. Literature Search and Data Extraction Analytes to Measure a Population's Health and Prosperity Status

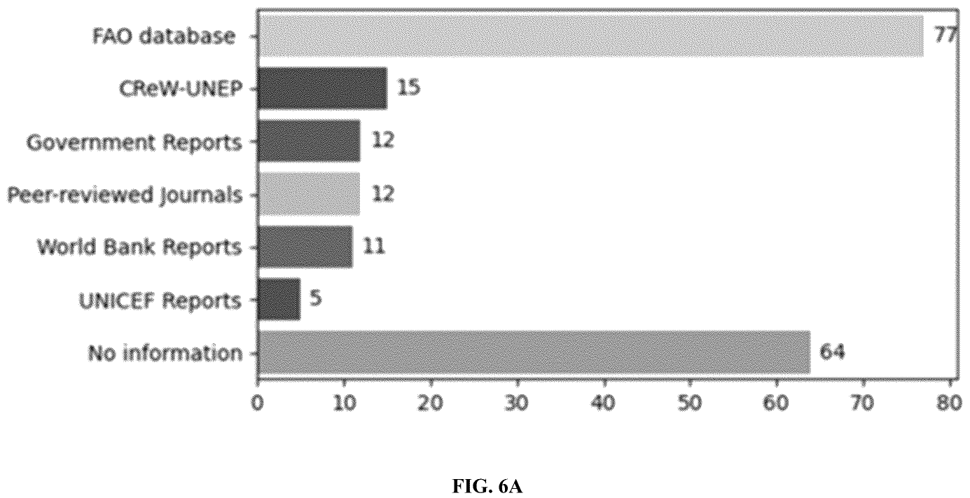

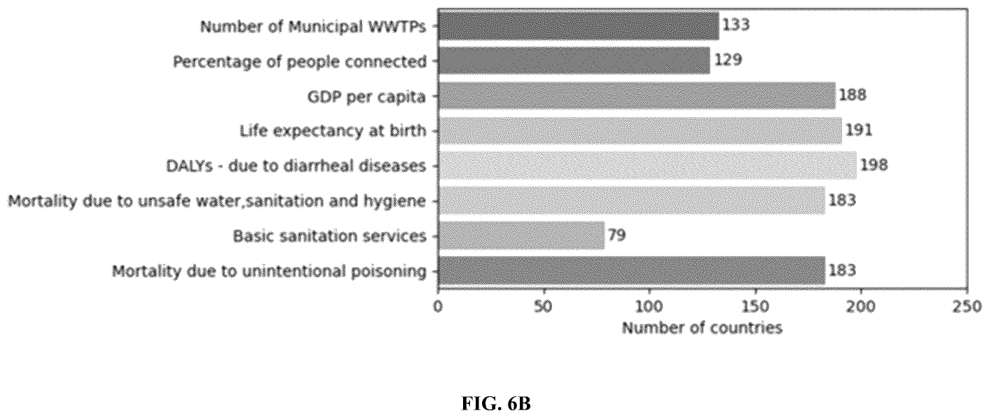

The United Nation's Food and Agricultural Organization (FAO) database, Web of Science, Science Direct, PubMed, and Google Scholar were used to find the number of wastewater treatment plants and the percentage of the people connected to municipal wastewater treatment plants in respective countries from 2013 using the Preferred Reporting Items for Systematic and Meta-Analysis (PRISMA) framework. One author of the team performed the initial screening and later replicated by a non-author of the team to confirm validity. Key phrase search criteria included “Municipal wastewater treatment plants in *,” “People connected with treatment plants in *,” “Number of centralized wastewater treatment plants in *,” “people connected with centralized treatment plants in *,” where * refers to each country listed in the UN. The search results were weighted by titles, abstracts, tables, and figures to determine the relevance of this study. Journal articles focusing on municipal wastewater treatment plants and wastewater-based epidemiology were included. All the sources of the data and indicators with the available data are shown in FIGS. 6A and 6B.

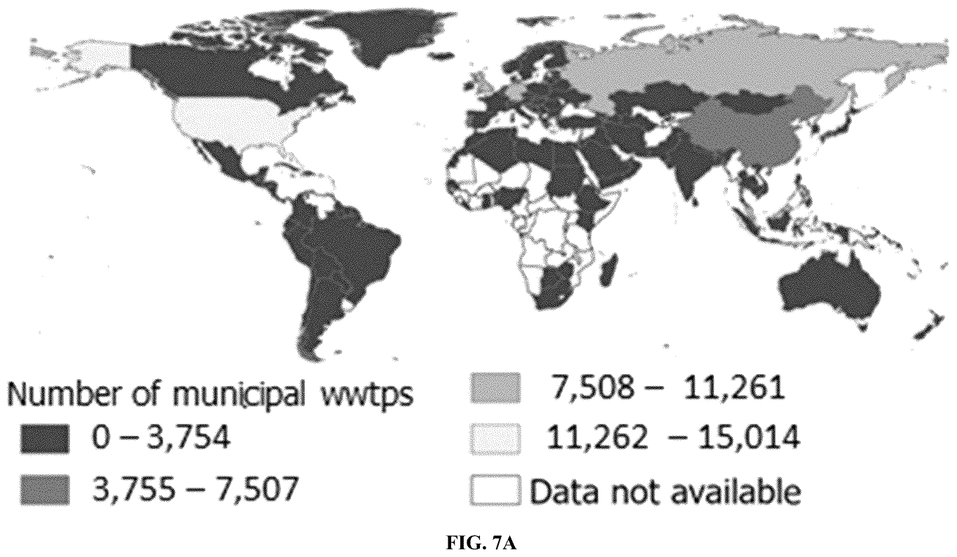

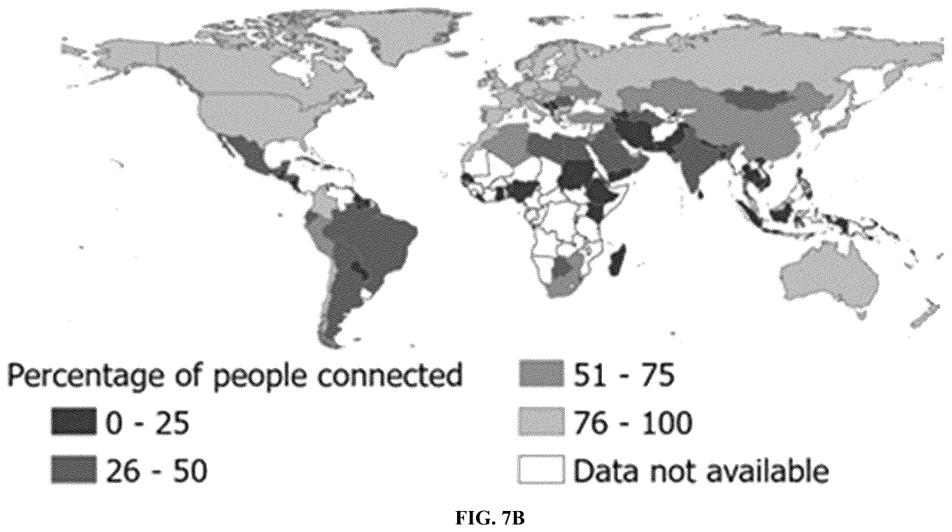

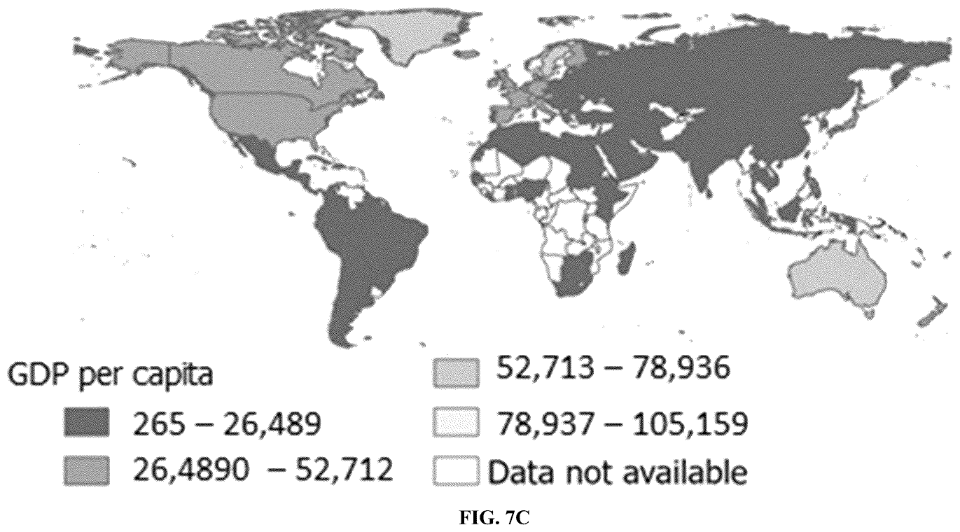

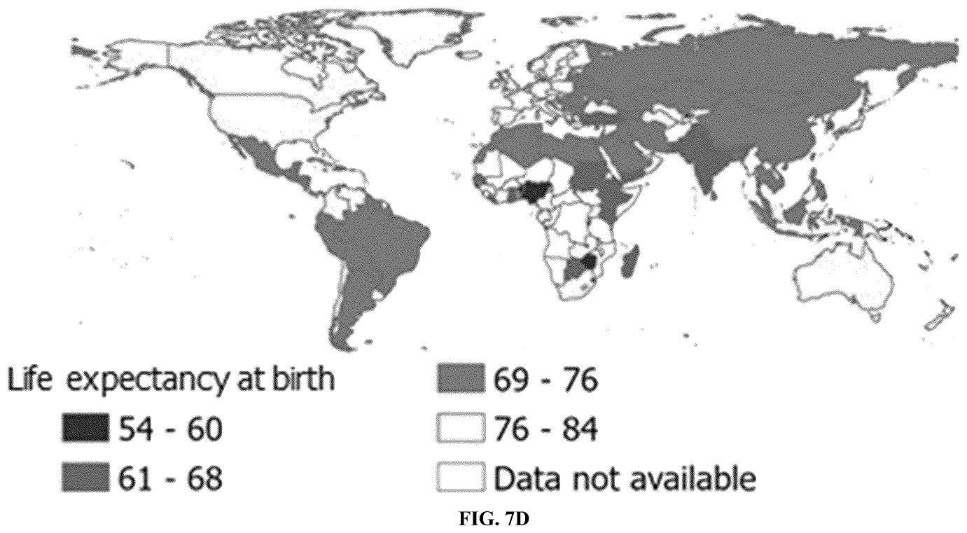

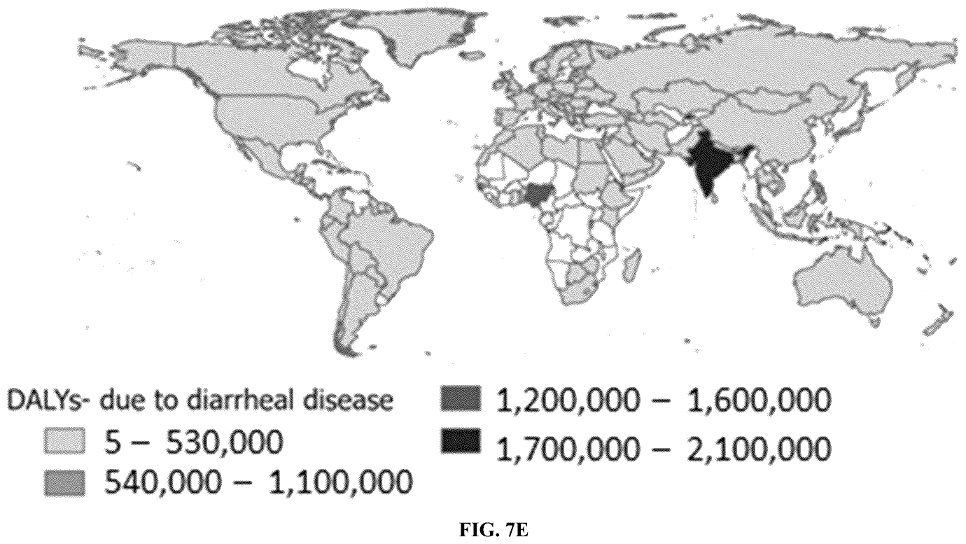

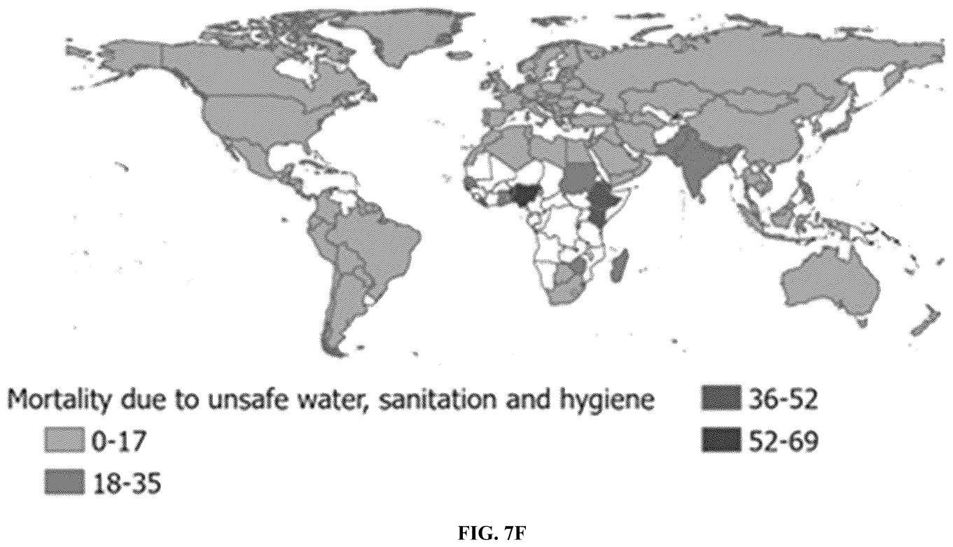

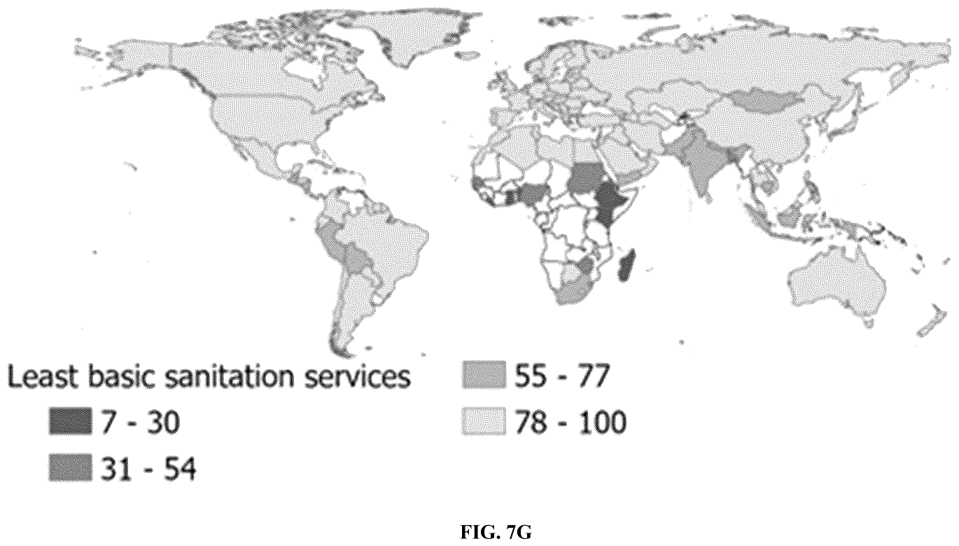

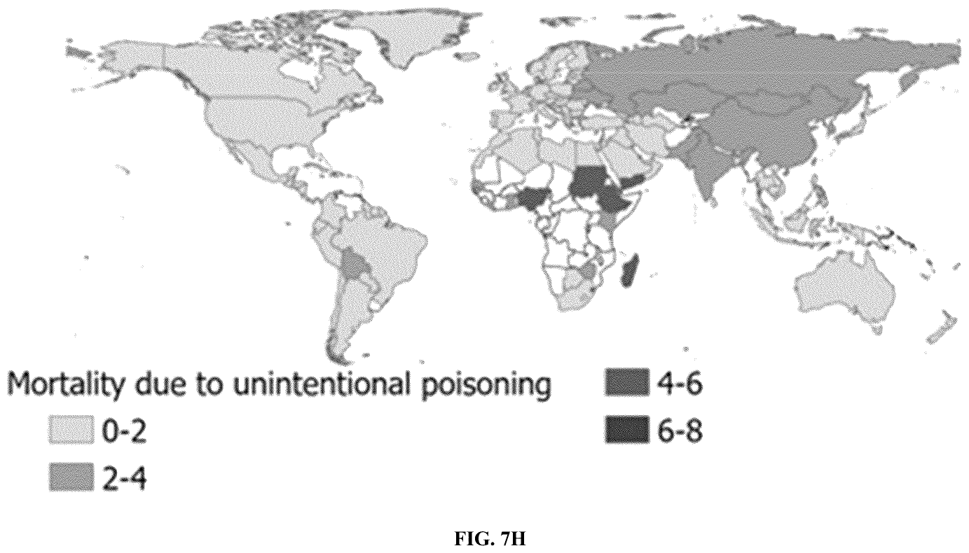

From available literature sources in the English language, some 109,159 wastewater treatment plants (WWTPs) globally were identified, representing 129 countries or 67% of all nations worldwide (Table 11). These plants receive biomarker-laden wastewater from approximately 2.7 billion people, which is equivalent to 34.7% of the world's 2021 population (FIGS. 7A and 7B).

TABLE 11
Inventory of global municipal wastewater treatment plants
and the fraction of the national population served.

| | | Population connected |
|---|---|---|
| Country | # of Municipal WWTPs | with WWTPs (%) |
| United States | 15014 | 76 |
| Germany | 9636 | 100 |
| Russian Federation | 9616 | 88 |
| Malaysia | 8291 | 78 |
| United Kingdom | 8035 | 98 |
| China | 4436 | 52 |
| Portugal | 4287 | 70 |
| France | 3275 | 100 |
| Poland | 3264 | 94 |
| Ukraine | 3093 | 51 |
| Brazil | 2800 | 26 |
| Italy | 2717 | 83 |
| Czech Republic | 2636 | 81 |
| Mexico | 2289 | 50 |
| Norway | 2240 | 99 |
| Japan | 2148 | 78 |
| Canada | 2064 | 82 |
| Spain | 2041 | 98 |
| Austria | 1842 | 100 |
| Sweden | 1243 | 100 |
| Australia | 1234 | 93 |
| Belgium | 1222 | 96 |
| Ireland | 1063 | 97 |
| South Africa | 923 | 57 |
| Denmark | 906 | 100 |
| Switzerland | 826 | 100 |
| India | 816 | 28 |
| Hungary | 739 | 77 |
| Latvia | 648 | 100 |
| Finland | 640 | 100 |
| Turkey | 604 | 64 |
| Estonia | 588 | 87 |
| Lithuania | 561 | 77 |
| South Korea | 505 | 92 |
| Romania | 481 | 43 |
| Colombia | 411 | 91 |
| Oman | 402 | 30 |
| Egypt | 382 | 40 |
| Israel | 376 | 97 |
| Nicaragua | 356 | 13 |
| Slovenia | 352 | 93 |
| Netherlands | 341 | 100 |
| New Zealand | 317 | 84 |
| Chile | 268 | 100 |
| Slovakia | 254 | 86 |
| Luxembourg | 25 | 99 |
| Greece | 235 | 93 |
| Jamaica | 187 | 64 |
| Peru | 184 | 57 |
| Belarus | 140 | 79 |
| Algeria | 138 | 65 |
| Zimbabwe | 137 | 66 |
| Iran | 129 | 18 |
| Mongolia | 115 | 28 |
| Croatia | 112 | 98 |
| Tunisia | 109 | 53 |
| Thailand | 101 | 21 |
| Dominican Republic | 91 | 35 |
| Bulgaria | 90 | 87 |
| Botswana | 75 | 39 |
| Morocco | 73 | 80 |
| Kazakhstan | 65 | 57 |
| Serbia | 53 | 12 |
| United Arab Emirates | 41 | 78 |
| Cyprus | 35 | 77 |
| Vietnam | 34 | 24 |
| Jordan | 32 | 62 |
| Philippines | 31 | 5 |
| Saudi Arabia | 30 | 50 |
| Guatemala | 27 | 5 |
| Kenya | 27 | 5 |
| Indonesia | 26 | 10 |
| Argentine | 25 | 43 |
| Libya | 23 | 43 |
| El Salvador | 21 | 3 |
| Iraq | 21 | 27 |
| Yemen | 20 | 3 |
| Georgia | 19 | 70 |
| Iceland | 18 | 57 |
| Bolivia | 15 | 40 |
| Bahrain | 11 | 88 |
| Bosnia and Herzegovina | 11 | 4 |
| Fiji | 11 | 40 |
| Honduras | 11 | 36 |
| Eswatini | 10 | 10 |
| Mauritius | 10 | 25 |
| Nepal | 10 | 2 |
| Nigeria | 10 | 4 |
| Cabo Verde | 9 | 21 |
| Paraguay | 9 | 9 |
| Senegal | 9 | 11 |
| Costa Rica | 8 | 12 |
| Cuba | 8 | 24 |
| Pakistan | 8 | 18 |
| Trinidad and Tobago | 8 | 25 |
| Albania | 7 | 8 |
| Ghana | 7 | 4 |
| Armenia | 6 | 61 |
| Azerbaijan | 6 | 19 |
| Bahamas | 6 | 15.6 |
| Kuwait | 6 | 100 |
| Qatar | 6 | 88 |
| Singapore | 6 | 100 |
| Malta | 4 | 100 |
| Micronesia | 4 | 17 |
| Montenegro | 4 | 18 |
| Panama | 4 | 55 |
| Sri Lanka | 4 | 14 |
| Belize | 3 | 15 |
| Cambodia | 3 | 11 |
| Ecuador | 3 | 48 |
| Guyana | 3 | 0 |
| Lebanon | 3 | 66 |
| Suriname | 3 | 10 |
| Barbados | 2 | 28 |
| Bhutan | 2 | 14 |
| Ethiopia | 2 | 18 |
| Liberia | 2 | 3 |
| Seychelles | 2 | 15 |
| Sudan | 2 | 10 |
| Bangladesh | 1 | 4 |
| Benin | 1 | 1 |
| Saint Lucia | 1 | 13 |
| Turkmenistan | 1 | 33 |
| Antigua and Barbuda | 0 | 0 |
| Burundi | 0 | 5 |
| Haiti | 0 | 0 |
| Madagascar | 0 | 0 |
| Saint Vincent and the Grenadines | 0 | 0 |

In high-income countries, about 80% of the people typically are connected to and served by municipal WWTPs, with the remaining 20% either using septic tanks or lacking sewage treatment infrastructure. Thirteen countries, including Austria, Chile, Denmark, Finland, France, Germany, Kuwait, Latvia, Malta, Netherlands, Singapore, Sweden, and Switzerland, have achieved complete or near-complete (˜100%) connectivity of their residents to some form of engineered centralized sewage treatment. For some 60 countries (FIG. 7A), data extraction was difficult due to a lack of information sources in the English language, resulting in poor geospatial coverage of Africa. Among those nations for which treatment infrastructure data was available (Antigua and Barbuda, the Bahamas, Barbados, Oman, Saudi Arabia, Saint Vincent and the Grenadines Seychelles, and Trinidad and Tobago), half the population or less are served by centralized wastewater systems. Notably, for Antigua and Barbuda, a nation classified in the high-income bracket, available data indicates complete absence of centralized municipal wastewater treatment. In upper-middle income countries, only 43% of the people were found to be connected to centralized wastewater treatment infrastructure. Among this economic bracket of nations, Colombia has the most people connected to centralized sewage collection and treatment, whereas existing infrastructure in Bosnia and Herzegovina and in Albania only reaches about 9% of the respective populations. The range of values determined for the Balkan region, however, showed a notable spread, ranging from 4% to 91%. In lower-income countries, on average about 24% of people are being served by centralized wastewater treatment systems (FIG. 7B). Eight percent of Moroccans are served by centralized sewage treatment, whereas only 3% of the population of El Salvador and Yemen enjoy this privilege. In low-income countries, as defined by the United Nations, an average of only 4% of people are served by centralized wastewater treatment, with Ethiopia ranking at the top of this group with a value of 19% (Table 11). For Haiti, not a single centralized wastewater treatment facility was documented. Most nations not included in this study (due to a lack of data) fall into the category of low-income and low-middle income countries.

Although the US has the largest number of WWTPs (15,014) by nation, the coverage of those plants (221 WWTPs per 1 million people) is moderate (76% of total US population), ranking 11th globally. By comparison, in China, which is similar in size by area, a lesser total of 4,287 WWTPs was found, which places this nation at rank #6 globally by total facility count. While the number of facilities is only a third of that in the US, China's infrastructure treats the sewage of more residents than the US does but still reaches only about 52% of its people (3 WWTPs per 1 million people), leading to a low rank of 82 globally. Most of the countries in Africa and South Asia rely on septic tanks, which do not readily lend themselves to the application of cost-effective, conventional WBE. Most rural areas in high-income and upper-middle-income countries also use septic systems, and information required for ranking often was not available. In the US, as in other parts of the world, the transition of rural populations into urban ones has brought a shift from an emphasis on decentralized to centralized sewage treatment systems.

### XIV. Statistical Analyses and Data Visualization for Health and Prosperity Status Analytes

Peer-reviewed articles which did not present data on the number of wastewater treatment plants within the manuscript or supplementary information were omitted from the analysis. All the databases and peer-reviewed journals were considered for this study if published in the English language. All available data was compiled in Microsoft Excel and analyzed using Python 3.8. Figures were created using a combination of Python 3.8 using Pycharm 2020.3 (Integrated Development Environment by JetBrains), ArcGIS-Pro 2.7.0, and Microsoft's Office Suite programs. Analyses were performed in Python version 3.8 using Pycharm 2020.3 (Integrated Development Environment by JetBrains). A pairwise correlation was performed on relevant infrastructure, economic, social, and environmental parameters categorically based on income level.

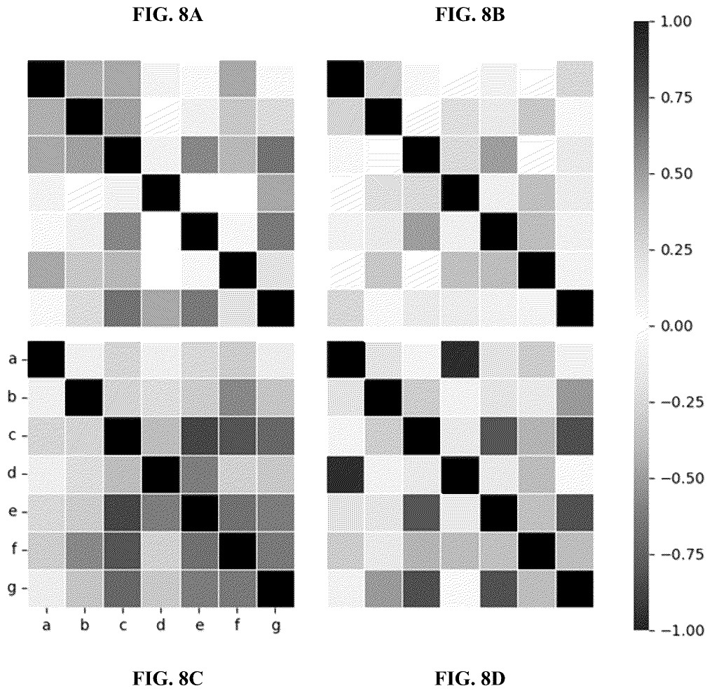

A Spearman correlation analysis was conducted to produce the heatmap shown in FIGS. 8A-8D, which relates access to centralized sewage collection and treatment to geography based on the income level. The corresponding Spearman correlation coefficients are tabulated in Table 12. People's access to centralized treatment of human waste was moderately related to GDP per capita (ρ=0.5) in high-income countries. In contrast, in low-middle income countries, a weaker correlation with national GDP was found (ρ=0.2). GDP per capita (FIG. 7C) and access to centralized sewage treatment showed an even weaker, negative correlation (ρ=−0.04) in low-income countries. As income levels decrease, it was found that the correlation coefficient (ρ) was decreasing, which may indicate that well-to-do people in high-income countries may prefer to live in rural areas, whereas the urban poor may have access to wastewater treatment, while otherwise being more disadvantaged. A lack of data on as-built infrastructure does not allow one to readily determine whether households in middle-low income and low-income countries use either a decentralized system for wastewater management or release human waste into the environment without any pre-treatment. Life expectancy at birth showed a moderate correlation with connectivity to municipal sewage treatment. The correlation between life expectancy at birth (FIG. 7D) and connectivity to WWTPs was moderate in high-income countries and even weaker in higher and lower-middle-income countries, respectively. It suggests that connectivity to centralized wastewater treatment is not indicative of life expectancy at birth. The availability of health care resources, availability of pharmaceutical products, skilled health care personnel, and life-related factors like consumption of alcohol and tobacco, and dietary habits like intake of sugar, caffeine, etc., mainly affect the life expectancy at birth. DALYs due to diarrheal diseases (FIG. 7E) and service by centralized WWTPs showed a strong positive correlation in these countries (ρ=0.6).

TABLE 12
Guide for interpreting the strength of Spearman
coefficient correlation coefficients.

| Spearman Coefficient Correlation (ρ) | Strength of Correlation |
|---|---|
| 0.00-0.19 | Very Weak |
| 0.20-0.39 | Weak |
| 0.40-0.59 | Moderate |
| 0.60-0.79 | Strong |
| 0.80-1.00 | Very Strong |

FIGS. 8A-8D demonstrate that indicators of sanitation are helpful in understanding and predicting disease incidence. Specifically, we observed that the burden of disease shows a negative correlation (−0.4≤ρ≤−0.04) with service by centralized wastewater infrastructure. This study used DALYs resulting from diarrheal diseases as an indicator to describe the burden of disease in the group of countries classified based on income. In high-income countries, it showed a weaker correlation between wastewater infrastructure and disease burden, possibly explainable by the use of decentralized sewage treatment and house latrines. A similar situation was observed in the group of upper-middle-income countries.

In the category of upper-middle income countries, the correlation between WWTP infrastructure and mortality-sanitation (FIG. 7F) was similar to that found for high-income countries. Botswana had a higher mortality from lack of sanitation (1.8/1000 people), whereas most of the countries in upper-middle income countries had a low mortality rate (0.1/1000 people). Data on mortality in the context of centralized wastewater treatment are rare or completely unavailable in lower-middle income countries and low-income countries, due in part to the use of decentralized systems. In general, a strong correlation between existing wastewater treatment plants and mortality from lack of sanitation was observed in lower-middle-income and low-income countries. The Spearman correlation was negative between access to centralized wastewater treatment and the mortality-sanitation for the category of lower-middle-income countries, thereby emphasizing the need of infrastructure investments to prevent the death caused by diseases from unsafe sanitation and hygiene. For example, this principally means that connectivity to wastewater treatment alone cannot explain the mortality due to sanitation, as inadequate drinking water, hand hygiene, and microbial community exposure are additional factors that contribute to the death of people from poor sanitation. In the category of low-income countries, it is evident that lack of centralized wastewater treatment shows a significant association with mortality- sanitation, and infrastructure investments in this region of the world are expected to translate into avoidance of deaths and extension of the lives of hundreds of thousands of people.

### XV. Collection and Measurement of Wastewater Samples for Assessing Exposure to VOCs

### a. Study Locations and Wastewater Sample Collection

Raw community wastewater samples were collected from two locations over four days in the southeastern US in May 2021. Community 1 has a population of approximately 25,000 people (83% White, 12% Black, 5% Hispanic) covering a geographic area of 20.6 km², which is within close proximity to manufacturing facilities and has poorer air quality than Community 2 with a population of 32,000 (87% White, 8% Black, 3% Hispanic, 2% other) and a geographic region of 87.8 km². Community 1 is in proximity to the industrial zone, therefore, is expected to have a higher concentration of VOCs in ambient air. Community 2 is far from the industrial zone and is expected to have relatively clean air. Unfortunately, due to confidentiality agreement with the local municipality, we are unable to provide the exact location of these sites. Wastewater samples were collected from automated samplers either from a maintenance hole (Community 1) or a local wastewater treatment plant location (Community 2). Aliquots were collected over a 24-h period (n=8 samples) and transferred to high-density polyethylene (HDPE) bottles for shipment to our lab at Arizona State University (Tempe, AZ). Samples were processed the same day upon the receipt to limit degradation losses.

### b. Chemicals and Reagents

LC-MS (liquid chromatography-mass spectrometry) grade methanol, acetonitrile, acetone, and water were purchased from Fisher Scientific (Thermo Fisher Scientific, Waltham, MA); LC-MS grade formic acid was purchased from Fluka Chemical Corp (Milwaukee, WI). High purity (>97%) reference standard and isotope-labeled standard solutions of the target compounds were obtained from Sigma Aldrich (Milwaukee, WI), C/D/N Isotopes Inc. (Pointe-Claire, Canada), and Toronto Research Chemicals (Toronto, Canada). Stock and working solutions of individual analytes were prepared in LC-MS grade water and were stored in amber glass vials with polytetrafluoroethylene (PTFE) septa at −18° C.

A list of all 35 reference standards and their respective stable isotope-labeled internal standards (SIL-IS) are given in Table 13. Analytes were selected based on a previous study examining the potential of WBE to assess population-level exposure to VOCs (Gracia-Lor et al., “Wastewater-based epidemiology as a novel biomonitoring tool to evaluate human exposure to pollutants.” Environ. Sci. Technol., 2018, 52(18): 10224-10226) and other studies that detected VOC metabolites in human urine (Alwis et al., “Simultaneous analysis of 28 urinary VOC metabolites using ultra high-performance liquid chromatography coupled with electrospray ionization tandem mass spectrometry (UPLC-ESI/MSMS).” Anal. Chim. Acta., 2012, 750: 152-160; Ding et al., “Simultaneous determination of six mercapturic acid metabolites of volatile organic compounds in human urine.” Chem. Res. Toxicol. 2009, 22(6): 1018-1025; Frigerio et al., “An LCMS/MS method to profile urinary mercapturic acids, metabolites of electrophilic intermediates of occupational and environmental toxicants.” J. Chromatogr., 2019, B 1117: 66-76; Tevis et al., “Harmonization of acronyms for volatile organic compound metabolites using a standardized naming system.” Int. J. Hyg. Environ. Health, 2021, 235: 113749). All selected analytes are urinary metabolites of VOCs exposure in humans, except nicotine.

TABLE 13
List of analytical targets indicative of VOC exposure in communities detected
as urinary metabolites in raw wastewater composited over 24-h.

| VOC | VOC metabolite | Harmonized Acronym |
|---|---|---|
| Acrolein | N-Acetyl-S-(2-carboxyethyl)-L-cysteine | 2CoEMA |
| | N-Acetyl-S-(3-hydroxypropyl)-L-cysteine | 3HPMA |
| Acrylamide | N-Acetyl-S-(2-carbamoylethyl)-L-cysteine | 2CaEMA |
| | N-Acetyl-S-(2-carbamoyl-2-hydroxyethyl)-L- | CaHEMA |
| | cysteine | |
| Acrylonitrile | N-Acetyl-S-(2-cyanoethyl)-L-cysteine | 2CyEMA |
| Ethylene Oxide/ | N-Acetyl-S-(2-hydroxyethyl)-L-cysteine | 2HEMA |
| Vinyl Chloride | | |
| Anabasine | Anabasine (free) | ANB |
| Anatabine | Anatabine (free) | ANTB |
| Benzene | N-Acetyl-S-(phenyl)-L-cysteine | PhMA |
| | trans, trans-Muconic acid | MUCA |
| 1-Bromopropane | N-Acetyl-S-(n-propyl)-L-cysteine | 1PMA |
| 1,3-Butadiene | N-Acetyl-S-(3,4-dihydroxybutyl)-L-cysteine | 34HBMA |
| | N-Acetyl-S-(1-hydroxymethyl-2-propenyl)-L- | 1HMPeMA/ |
| | cysteine/N-Acetyl-S-(2-hydroxy-3-butenyl)-L- | 2HBeMA |
| | cysteine | |
| | N-Acetyl-S-(4-hydroxy-2-buten-1-yl)-L-cysteine | (E)-4HBeMA |
| Carbon disulfide | 2-Thioxothiazolidine-4-carboxylic acid | TTCA |
| Crotonaldehyde | N-Acetyl-S-(3-hydroxypropyl-1-methyl)-L-cysteine | 3HMPMA |
| Cyanide | 2-Aminothiazoline-4-carboxylic acid | 2ATCA |
| N, N- | N-Acetyl-S-(N-methylcarbamoyl)-L-cysteine | MCaMA |
| Dimethylformamide | | |
| Ethylbenzene | Phenylglyoxylic acid | PhGA |
| Nicotine | Nicotine | NIC |
| | Cotinine | COT |
| | 3-Hydroxycotinine | 3HC |
| Propylene oxide | N-Acetyl-S-(2-hydroxypropyl)-L-cysteine | 2HPMA |
| Styrene | N-Acetyl-S-(1-phenyl-2-hydroxyethyl)-L-cysteine+ | 2HPhEMA |
| | Mandelic acid | MADA |
| VOC | VOC metabolite | Harmonized Acronym |
| Tetrachloroethylene | N-Acetyl-S-(trichlorovinyl)-L-cysteine | 122CVMA |
| Toluene | N-Acetyl-S-(benzyl)-L-cysteine | BzMA |
| Trichloroethylene | N-Acetyl-S-(1,2-dichlorovinyl)-L-cysteine | 12CVMA |
| | N-Acetyl-S-(2,2-dichlorovinyl)-L-cysteine | 22CVMA |
| Xylene | N-Acetyl-S-(2,4-dimethylphenyl)-L-cysteine | 24MPhMA |
| | N-Acetyl-S-(2,5-dimethylphenyl)-L-cysteine | 25MPhMA |
| | N-Acetyl-S-(3,4-dimethylphenyl)-L-cysteine | 34MPhMA |
| | 2-Methylhippuric acid | 2MHA |
| | 3-Methylhippuric acid | 3MHA |
| | 4-Methylhippuric acid | 4MHA |

### c. Sample Processing

Duplicate 100 mL aliquots of raw, unfiltered wastewater samples were acidified to pH 2 using formic acid and spiked with a mixture of stable isotope-labeled (deuterium, ¹³C, or ¹⁵N) internal standards at a concentration of 5 ng mL⁻¹ and extracted using a Dionex™ AutoTrace™ 280 Solid-Phase Extraction (SPE) Instrument (Thermo Scientific, Waltham, MA) with Hydrophilic-Lipophilic Balance (HLB) cartridges (6 cc, 150 mg, 30 μm particle size) manufactured by Waters (Milford, MA). Isotope labeled internal standards were added to wastewater samples after acidification and prior to processing. SPE Cartridges were conditioned with 5 mL of acidified LC-MS grade water (pH 2) and 5 mL of methanol before sample loading. Subsequently, cartridges were rinsed with 5 mL of acidified LC-MS grade water (pH 2) and dried under a stream of nitrogen. Target analytes were extracted with 2 mL of 50:50 (v/v) methanol and acetone containing 0.5% formic acid into an amber glass vial via dropwise elution of cartridges. Finally, 200 μL of the final extract was dried overnight at ambient temperatures and reconstituted in 100% LC-grade water for analysis. Analyses were performed using triple quadrupole liquid chromatography-tandem mass spectrometry (LC-MS/MS). The number of freeze-thaw cycles of the VOC metabolite standards were ≤3, following the Centers for Disease Control and Prevention (CDC) suggestion to prevent degradation.

### d. Instrumentation and Method Parameters

Wastewater extracts were analyzed on a Shimadzu LCMS 8060 with an electrospray ionization (ESI) interface coupled to Shimadzu Nexera™ X2 ultra-HPLC (Shimadzu Scientific, Kyoto, Japan). The chromatographic separation of analytes was completed on a 50×2.1 mm, 1.8 μm particle size Raptor™ biphenyl column (Restek, USA) at a 0.2 mL min⁻¹ flow rate. The binary gradient elution was performed with mobile phases consisting of LC-grade water (A) and methanol (B). The optimized gradient of 8 min is as follows: 5% mobile phase B for 1 min with a linear increase from 5% to 65% until 3.0 min; followed by an increase in the gradient to 95% at 3.1 min; held at 95% B for a further 3.0 min and returned to the initial gradient of 10% B. The injection volume was 5 μL, and the column oven temperature was set at 35° C.

High purity argon was used as collision-induced dissociation gas, and nitrogen and dry air were generated using a Proton Onsite DualGas D251M LCMS gas generator (Proton Onsite, USA). The ESI was operated in both positive and negative ionization mode, and the source parameters were as follows: Nebulizing gas flow=3 L min⁻¹, heating gas flow=10 L min⁻¹, drying gas flow=10 L min⁻¹, interface temperature=300° C., desolvation temperature=526° C., desolvation line temperature=250° C., heat block temperature=400° C. The MS parameters for target analytes and labeled standards are provided in Table 14.

TABLE 14
MRM transitions of analytes included in the study.

| | | MRM transition | MRM transition | Retention | Q1 Pre- | | Q3 Pre- | MRM |
|---|---|---|---|---|---|---|---|---|
| VOC metabolite | Type | (Quantifier ion) | (Qualifier ion) | time (min) | Bias (V) | CE (V) | Bias (V) | mode |
| 2CoEMA | Target | 234.2 > 105.2 | 234.2 > 162.3 | 0.60 | 12, 12 | 10, 15 | 30, 40 | − |
| 2CoEMA-d₃ | ISTD | 237 > 105.2 | No Qualifier ion | 0.60 | 16 | 15 | 22 | − |
| 3HPMA | Target | 220 > 91.1 | 220 > 89.1 | 0.77 | 18, 19 | 26, 13 | 17, 17 | − |
| 3HPMA-d₃ | ISTD | 223.1 > 91.1 | No Qualifier ion | 0.78 | 30 | 14 | 36 | − |
| 2CaEMA | Target | 233 > 104.2 | 233 > 162.3 | 0.60 | 27, 21 | 12, 17 | 17, 19 | − |
| 2CaEMA-d₄ | ISTD | 237 > 108.2 | 237 > 163.3 | 0.60 | 17, 29 | 11, 14 | 31, 43 | − |
| 1CaHEMA | Target | 249.2 > 120.2 | 249.2 > 128.2 | 0.57 | 30, 30 | 10, 15 | 24, 22 | − |
| 1CaHEMA-d₃ | ISTD | 252.1 > 120.2 | 252.1 > 131.2 | 0.57 | 24, 16 | 10, 15 | 25, 23 | − |
| 2CyEMA | Target | 215.1 > 86.1 | 215.1 > 162.3 | 0.94 | 23, 24 | 7, 12 | 32, 36 | − |
| 2CyEMA-d₃ | ISTD | 218.1 > 165.3 | 218.1 > 86.1 | 0.95 | 15, 18 | 12, 7 | 34, 32 | − |
| 2HEMA | Target | 206.1 > 77 | 206.1 > 75 | 0.60 | 18, 25 | 25, 13 | 29, 33 | − |
| 2HEMA-d₄ | ISTD | 210.2 > 81.2 | No Qualifier ion | 0.60 | 23 | 12 | 15 | − |
| ANB | Target | 163.1 > 92.3 | 163.1 > 118.3 | 4.24 | −18, −14 | −22, −28 | −22, −17 | + |
| ANB-d₄ | ISTD | 167.1 > 96.3 | 167.1 > 134.4 | 4.22 | −13, −13 | −29, −23 | −18, −18 | + |
| ANTB | Target | 161.1 > 117.3 | 161.1 > 143.4 | 4.1 | −28, −28 | −17, −23 | −18, −22 | + |
| ANTB-d₄ | ISTD | 165.1 > 148.4 | No Qualifier ion | 4.1 | −15 | −20 | −28 | + |
| PhMA | Target | 238.1 > 109.2 | 239.1 > 110.2 | 3.6 | 28, 20 | 23, 22 | 20, 20 | − |
| PhMA-d₅ | ISTD | 243.2 > 114.2 | No Qualifier ion | 3.5 | 28 | 23 | 20 | − |
| MUCA | Target | 141.1 > 53.2 | No Qualifier ion | 0.56 | 28 | 12 | 22 | − |
| MUCA-d₄ | ISTD | 145 > 101.1 | No Qualifier ion | 0.55 | 26 | 10 | 19 | − |
| 1PMA | Target | 204.2 > 75.1 | 204.2 > 84.2 | 1.98 | 24, 24 | 11, 22 | 34, 30 | − |
| 1PMA-d₇ | ISTD | 211 > 82.2 | 211 > 84.2 | 1.90 | 26, 24 | 10, 23 | 17, 16 | − |
| 34HBMA | Target | 250.1 > 121.2 | 250.1 > 75.1 | 0.63 | 16, 20 | 27, 17 | 31, 23 | − |
| 34HBMA-d₇ | ISTD | 257.1 > 128.2 | No Qualifier ion | 0.64 | 30 | 15 | 13 | − |
| 1HMPeMA/2HBeMA | Target | 232.1 > 103.2 | No Qualifier ion | 1.13 | 26 | 13 | 21 | − |
| 1HMPeMA/2HBeMA-d₆ | ISTD | 238.1 > 109.2 | No Qualifier ion | 3.55 | 28 | 14 | 44 | − |
| (E)-4HBeMA | Target | 232.1 > 103.2 | 232.1 > 85.1 | 1.13 | 27, 21 | 25, 13 | 16, 20 | − |
| (E)-4HBeMA-d₃ | ISTD | 235.1 > 103.2 | 235.1 > 85.1 | 1.13 | 17, 17 | 25, 15 | 37, 41 | − |
| TTCA | Target | 161.9 > 58.1 | 163.1 > 33.1 | 0.64 | 19, 19 | 27, 12 | 32, 24 | − |
| TTCA-₁₃C₃ | ISTD | 165.1 > 58.1 | No Qualifier ion | 0.65 | 15 | 10 | 24 | − |
| 3HMPMA | Target | 234.1 > 105.2 | No Qualifier ion | 1.20 | 20 | 14 | 20 | − |
| 3HMPMA-d₃ | ISTD | 237 > 105.2 | No Qualifier ion | 1.16 | 28 | 13 | 40 | − |
| 2ATCA | Target | 145 > 67.2 | No Qualifier ion | 0.81 | 36 | 12 | 28 | − |
| 2ATCA-¹³C₁₅, N₂ | ISTD | 149.1 > 131.2 | No Qualifier ion | 0.70 | 34 | 15 | 20 | − |
| MCaMA | Target | 219.1 > 162.3 | No Qualifier ion | 0.66 | 27 | 7 | 32 | − |
| MCaMA-d₃ | ISTD | 222.1 > 165.3 | 222.1 > 87.2 | 0.70 | 17, 19 | 15, 10 | 17, 16 | − |
| PhGA | Target | 149 > 77.2 | 149.1 > 105.2 | 1.0 | 20, 18 | 12, 12 | 42, 30 | − |
| PhGA-d₅ | ISTD | 154 > 82.2 | No Qualifier ion | 1.0 | 32 | 13 | 15 | − |
| NIC | Target | 163.1 > 117.3 | 163.1 > 130.3 | 4.0 | −12, −12 | −20, −28 | −24, −21 | + |
| NIC-d₃ | ISTD | 166.1 > 117.3 | 166.1 > 130.3 | 4.0 | −20, −19 | −20, −25 | −24, −22 | + |
| COT | Target | 177.1 > 80.3 | 177.1 > 98.3 | 4.3 | −18, −16 | −21, −25 | −18, −14 | + |
| COT-d₃ | ISTD | 180.1 > 80.3 | 180.1 > 100.4 | 4.3 | −16, −14 | −21, −28 | −48, −14 | + |
| 3HC | Target | 193.1 > 80.3 | 193.1 > 134.3 | 3.86 | −22, −15 | −20, −30 | −14, −25 | + |
| 3HC-d₃ | ISTD | 196.1 > 80.3 | 196.1 > 61.4 | 3.86 | −16, −17 | −35, −25 | −11.3, −14.5 | + |
| 2HPMA | Target | 220.2 > 91 | 220.2 > 89 | 0.77 | 16, 23 | 27, 13 | 14, 17 | −− |
| 2HPMA-d₃ | ISTD | 223.1 > 91.1 | No Qualifier ion | 0.75 | 26 | 12 | 16 | − |
| 2HPhEMA | Target | 282.1 > 153.2 | 282.1 > 123.2 | 3.27 | 19, 20 | 22, 12 | 22, 32 | − |
| 2HPhEMA-d₃ | ISTD | 285.1 > 153.2 | 285.1 > 123.2 | 3.26 | 14, 14 | 24, 14 | 22, 16 | − |
| MADA | Target | 151.1 > 107.2 | No Qualifier ion | 0.90 | 20 | 13 | 20 | − |
| MADA-d₅ | ISTD | 156 > 112.2 | No Qualifier ion | 0.90 | 20 | 13 | 22 | − |
| 122CVMA | Target | 290 > 161.1 | 290 > 35.1 | 3.68 | 42, 22 | 25, 10 | 40, 32 | − |
| 122CVMA-d₃ | ISTD | 295 > 163.1 | No Qualifier ion | 3.68 | 34 | 10 | 30 | − |
| BzMA | Target | 252.1 > 123.2 | No Qualifier ion | 3.79 | 22 | 15 | 24 | − |
| BzMA-d₃ | ISTD | 255.2 > 123.2 | No Qualifier ion | 3.78 | 30 | 15 | 23 | − |
| 12CVMA | Target | 256.1 > 120.2 | No Qualifier ion | 3.34 | 27 | 7 | 24 | − |
| 12CVMA-¹³C d₃ | ISTD | 261.1 > 127.1 | No Qualifier ion | 3.34 | 17 | 6 | 23 | − |
| 22CVMA | Target | 256.1 > 127.1 | 256.1 > 91.1 | 3.20 | 32, 30 | 31, 11 | 17, 24 | − |
| 22CVMA-¹³C d₃ | ISTD | 260 > 127.1 | No Qualifier ion | 3.34 | 18 | 12 | 25 | − |
| 24MPhMA | Target | 266.2 > 137.2 | 266.2 > 136.2 | 4.16 | 18, 22 | 40, 18 | 14, 25 | − |
| 24MPhMA-d₃ | ISTD | 269.1 > 137.2 | No Qualifier ion | 4.16 | 22 | 16 | 25 | − |
| 25MPhMA-d₃ | ISTD | 269.1 > 137.3 | No Qualifier ion | 4.16 | 20 | 18 | 28 | − |
| 25MPhMA | Target | 266.1 > 137.3 | No Qualifier ion | 4.16 | 32 | 23 | 28 | − |
| 34MPhMA | Target | 266.2 > 137.3 | 266.2 > 136.3 | 4.16 | 20, 22 | 37, 17 | 26, 26 | − |
| 34MPhMA-d₃ | ISTD | 269.1 > 137.2 | No Qualifier ion | 4.16 | 24 | 17 | 26 | − |
| 2MHA | Target | 192.1 > 148.3 | 192.1 > 91.2 | 3.38 | 21, 23 | 16, 14 | 36, 28 | − |
| 2MHA-d₇ | ISTD | 199.1 > 155.4 | 199.1 > 97.2 | 3.34 | 17, 14 | 17, 14 | | − |
| 3MHA | Target | 192.1 > 148.3 | 192.1 > 91.2 | 3.38 | 23, 22 | 17, 14 | 17, 15 | − |
| 3MHA-d₇ | ISTD | 199 > 155.3 | 199 > 98.2 | 3.35 | 40, 26 | 16, 14 | 17, 16 | − |
| 4-MHA | Target | 192.1 > 148.3 | 192.1 > 91.2 | 3.38 | 21, 23 | 17, 15 | 18, 15 | − |
| 4MHA-d₇ | ISTD | 198.1 > 155.4 | 199.1 > 98.2 | 3.27 | 22, 23 | 17, 13 | 18, 30 | − |

Raw data were acquired by LabSolutions™ (version 5.93, Shimadzu, Kyoto, Japan) and processed using Lab Solutions Insight (version 3.2, Shimadzu). Method development and multiple reaction monitoring (MRM) optimization for each analyte were performed via flow injection (without an analytical column).

### e. Quantification of Analytes and Method Validation

Analytes were quantified using the isotopic dilution method, where analyte responses (peak area) were proportioned to the responses of corresponding mass-labeled standards. Analytes were identified using their specific retention time and MRM transitions. Linearity was evaluated over seven-point calibration ranging from 0.001 μg L⁻¹-20 μg L⁻¹ analyzed in triplicates. The calibration was performed by analyzing analytical standards prepared in LC-grade water. The concentration of analytes in wastewater extracts was quantified only for those with a signal-to-noise ratio (S/N)≥10. Qualitative detections were assessed for those compounds with a signal-to-noise ratio of 3≤(S/N)≥10. Interday and intraday variation in the instrument performance was assessed by analyzing a set of standards multiple times a day on three different days. One instrument blank was analyzed with each set of 10 wastewater samples, and a set of method blanks were processed in the same manner as samples and analyzed to determine any background contamination in the analytical procedure. Recoveries were assessed by comparing the peak area of analytes spiked in ultrapure water at two concentrations (1 μg L⁻¹ and 5 μg L⁻¹), to the analytical standards prepared in LC-grade water. Limit of detection (LOD) and limit of quantification (LOQ) were determined by a S/N ratio of 3 and 10, respectively.

### XVI. Method Performance

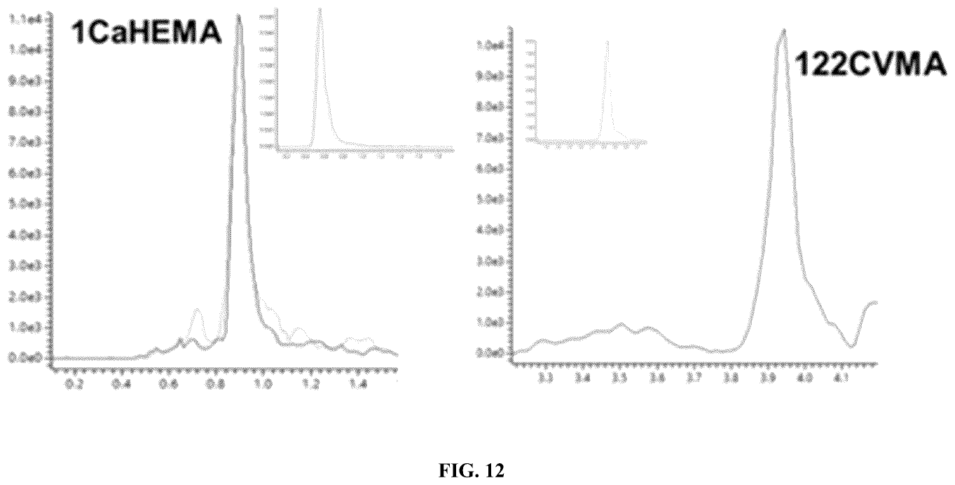

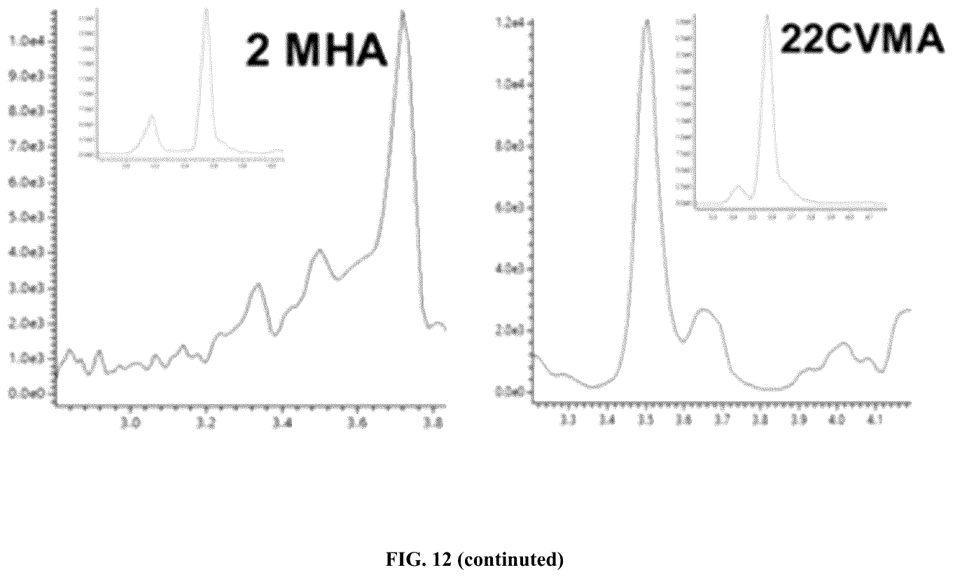

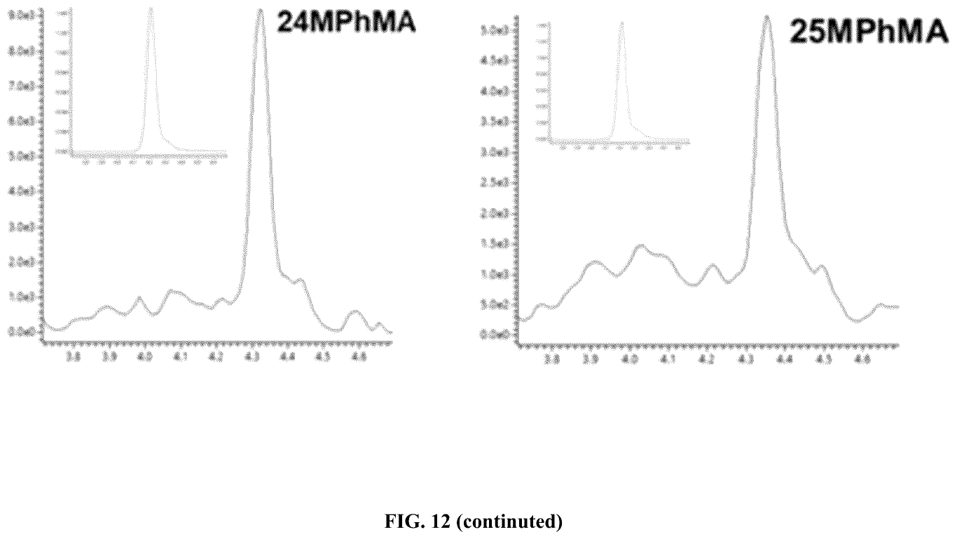

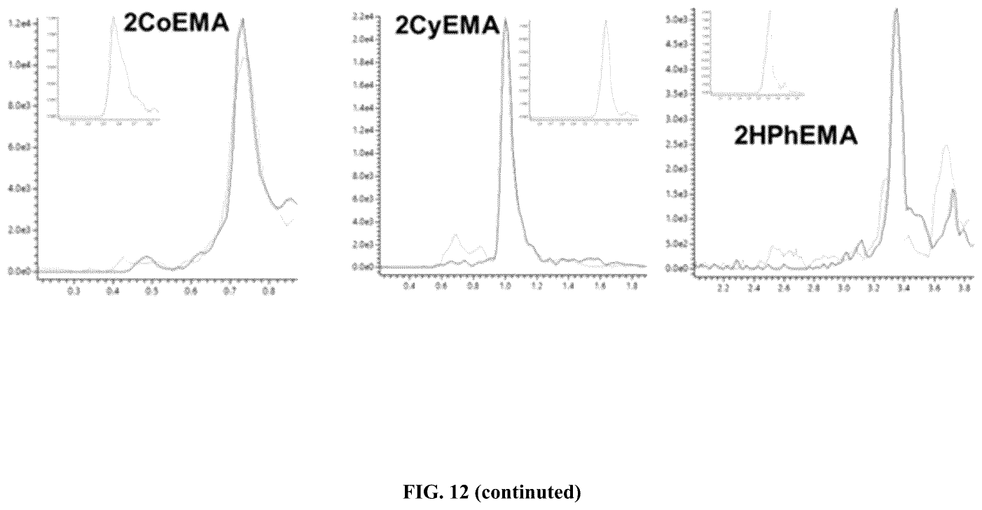

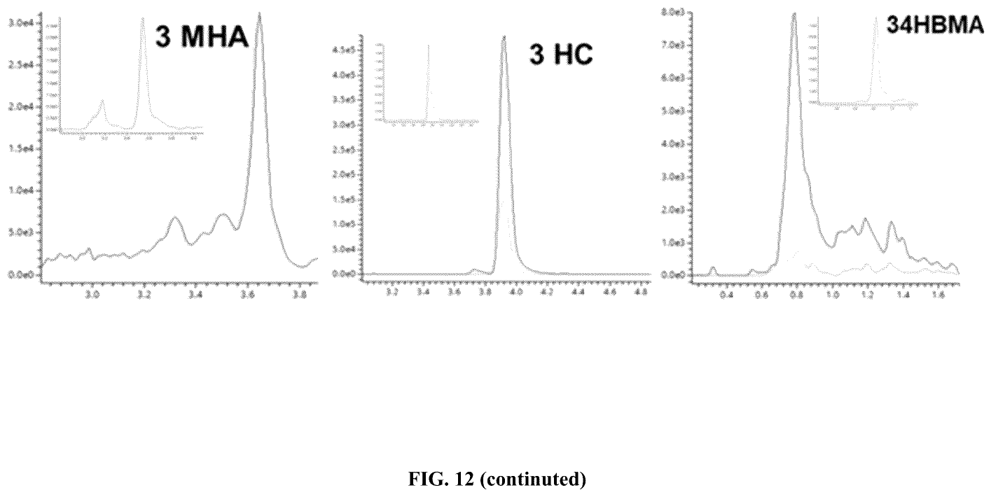

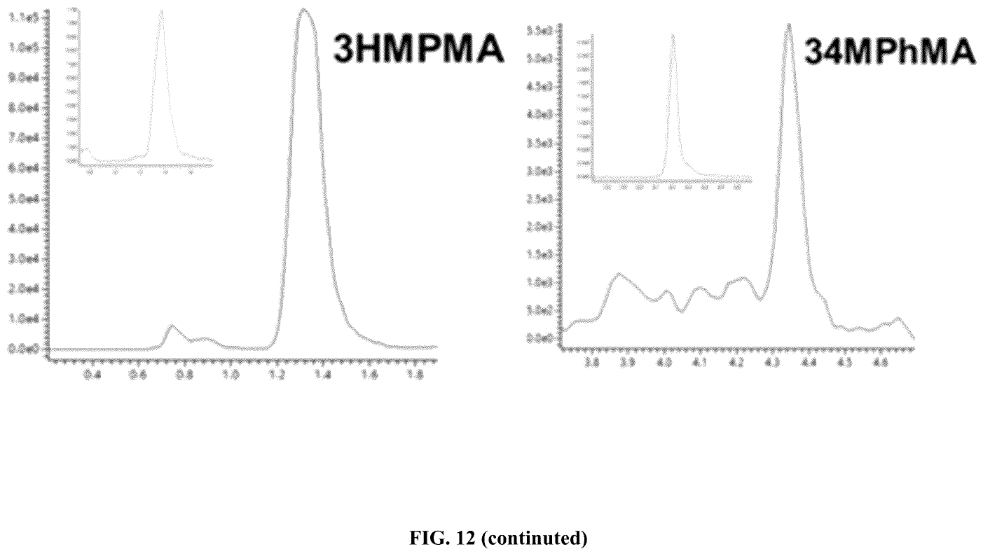

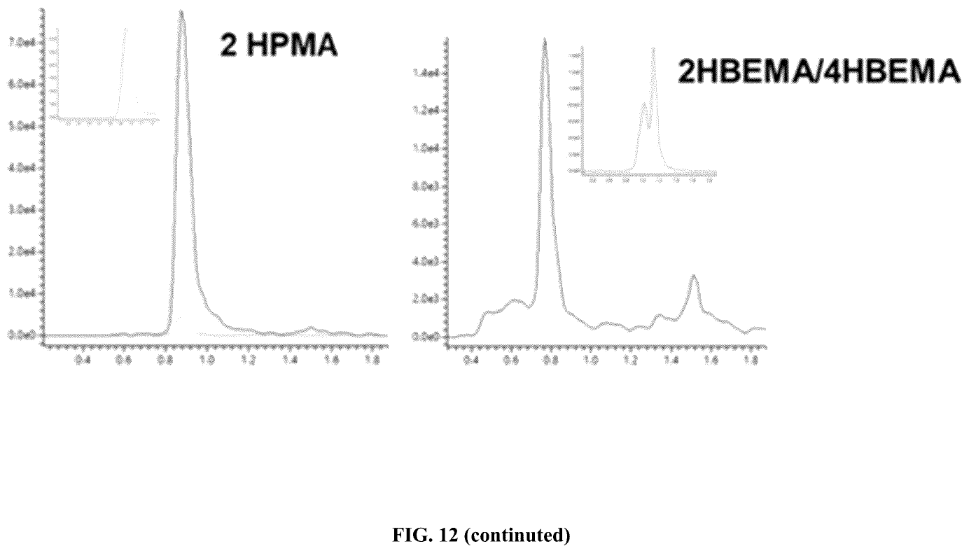

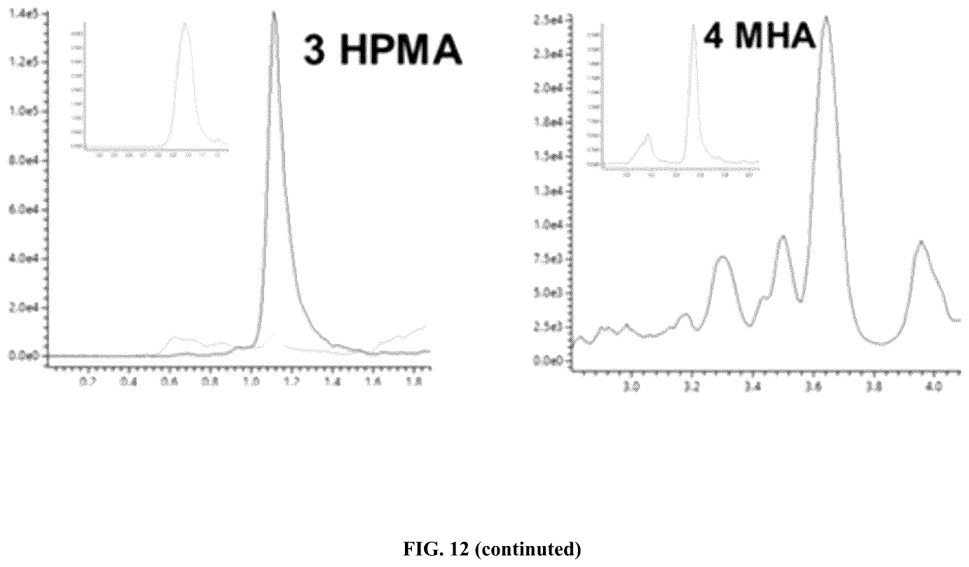

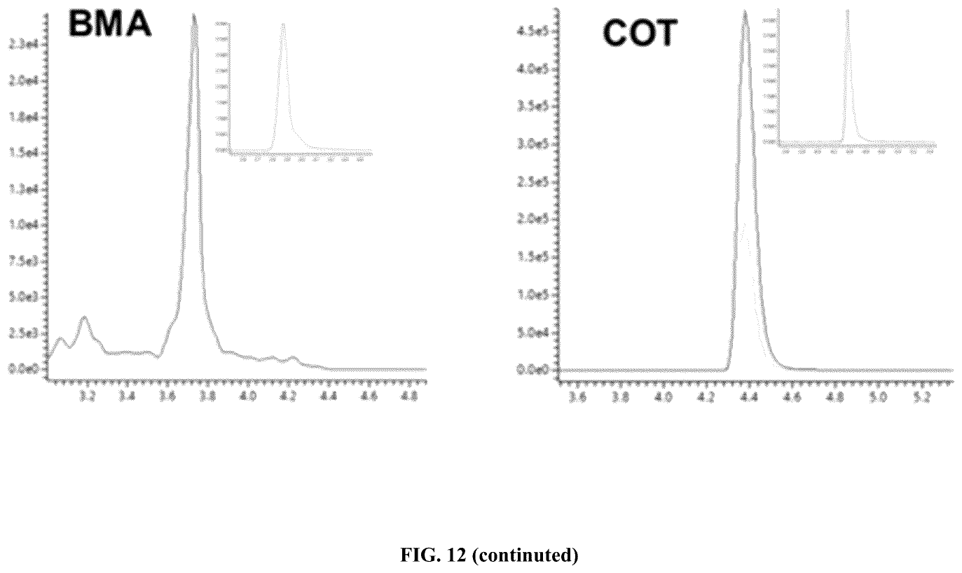

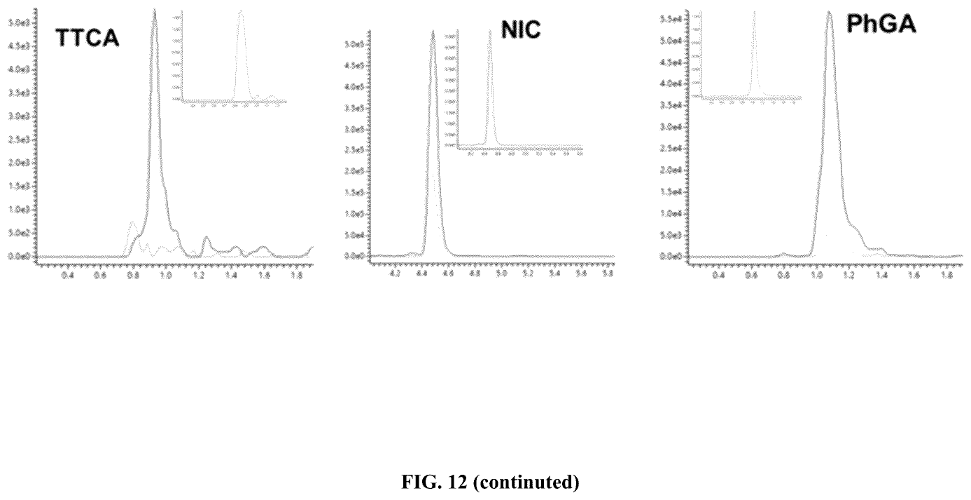

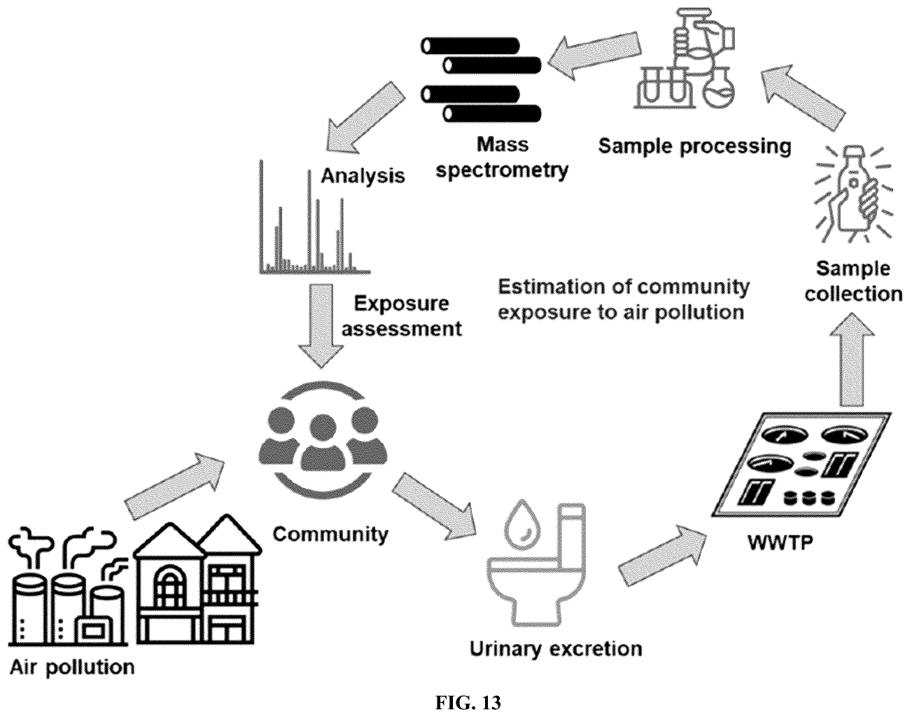

Concentrations of VOC isomeric metabolites (2MHA, 3MHA, 4MHA; 2HBEMA, 4HBEMA; 2HPMA, 3HPMA) with identical Multiple Reaction Monitoring (MRM) transitions and retention times were shown as a summation. Linearity of the calibration was obtained for all studied analytes with the coefficient of regression, r²≥0.99. The precision of the analysis was measured using relative percentage differences (RPD) between sample duplicates, and only those with RPD <30% were reported. The interday and intraday variation in the peak area of analyte chromatograms was <10%. The retention time of analytes in the wastewater matrix was within ±0.2 min of the retention time of the standard compound under the same conditions. The chromatograms of analytes detected in raw wastewater are shown in FIG. 12. The unseparated chromatograms of isomers of HPMA, HBeMA, and MHA are one of the analytical limitations of this study. Analytes were not detected in any instrument blank or method blank. There was an insignificant difference (<10%) between the peak area of analytes spiked in ultrapure water (with subsequent full method processing) and the peak area of the analytical standard of the same concentration, including those eluting close to the column void time, illustrating method robustness. LOD and LOQ ranged from 1 to 30 ng L⁻¹, and 3 to 100 ng L⁻¹ (Tables 15 and 14), respectively.

TABLE 15
Limit of detection (LOD) and limit of quantification
(LOQ) of analytes detected in community wastewater.

| Analyte | LOD (ng/L) | LOQ (ng/L) |
|---|---|---|
| 2CoEMA | 10 | 30 |
| 3HPMA | 10 | 30 |
| 2CyEMA | 5 | 15 |
| 34HBMA | 10 | 30 |
| ΣHBeMA | 3 | 10 |
| 3HMPMA | 30 | 90 |
| MCaMA | 3 | 10 |
| PhGA | 10 | 30 |
| NIC | 30 | 100 |
| COT | 10 | 30 |
| 3HC | 10 | 30 |
| ΣHPMA | 30 | 90 |
| 2HPhEMA | 1 | 3 |
| 122CVMA | 3 | 10 |
| BzMA | 1 | 3 |
| ΣMHA | 10 | 30 |

### XVII. Occurrence of VOCs Metabolites in Community Wastewater

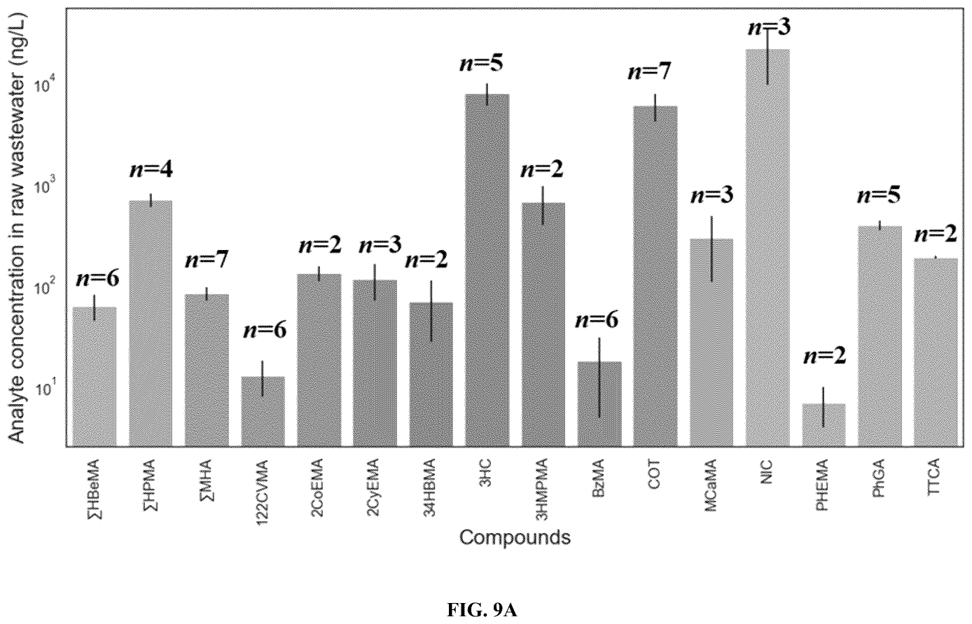

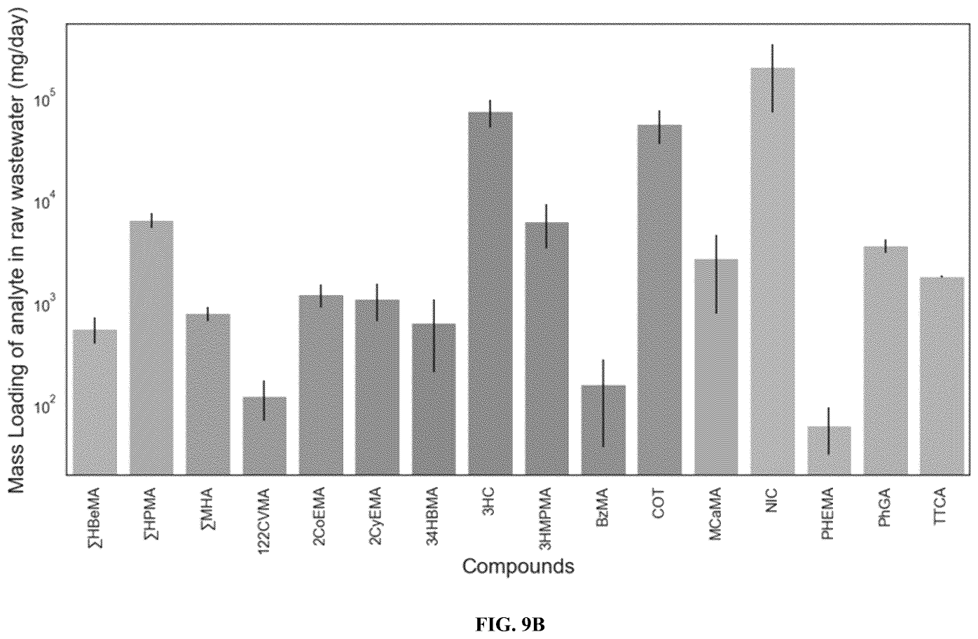

Eight raw wastewater samples were collected from two communities in the southeastern US, with varying degrees of outdoor air pollution, to assess whether VOC metabolites of interest could be i) measured in community wastewater samples, and ii) determine differences, if any, between VOC levels in the two communities. Of the 35 analytes analyzed in the present study, 16 were detected within the quantifiable range at least once. The average concentration and mass load of VOC metabolites in community wastewater ranged from 7 ng L⁻¹-2600 ng L⁻¹ and 62 mg day⁻¹-227,400 mg day⁻¹, respectively. Detection frequencies of quantifiable VOC metabolites are shown in FIGS. 9A and 9B and Table 16. The most frequently detected analytes included cotinine (nicotine) and ΣHBEMA (1,3-butadiene) with 88% detection frequency (DF), and ΣHPMA (acrolein, propylene oxide), 122CVMA (tetrachloroethylene), BzMA (toluene) with 75% detection DF. The average mass load of VOC analytes ranked in the decreasing order included: nicotine, 3HC, COT (nicotine-related compounds); ΣHPMA (acrolein, propylene oxide); 3HMPMA (crotonaldehyde); PhGA (ethylbenzene); MCaMA (n,n-dimethylformamide); TTCA (carbon disulfide); 2C oEMA (acrolein); 2CyEMA (acrylonitrile); ΣMHA (xylenes); Σ2HBEMA/4HBEMA (1,3-butadiene) and 34HBMA (1,3-butadiene); BzMA (toluene); 122CVMA (tetrachloroethylene); and 2HPhEMA (styrene). Five additional metabolites were qualitatively detected, including 2CaHEMA (acrylamide), 22CVMA (trichloroethylene), 24MPhMA (xylene), and the nicotine-related alkaloids anabasine (ANB) and anatabine (ANTB).

TABLE 16
Average concentration and detection frequencies of
analytes detected in raw wastewater (n = 8),
collected from two US communities during May 2021.

| Analyte | Average Concentration (ng/L) | Detection Frequency (%) |
|---|---|---|
| 2CoEMA | 134 ± 23 | 25 |
| 3HPMA | 742 ± 411 | 63 |
| 2CyEMA | 117 ± 46 | 38 |
| 34HBMA | 70 ± 42 | 25 |
| ΣHBeMA | 63 ± 18 | 88 |
| 3HMPMA | 679 ± 283 | 88 |
| MCaMA | 300 ± 191 | 38 |
| PhGA | 398 ± 46 | 63 |
| NIC | 22,333 ± 12,702 | 38 |
| COT | 6046 ± 1869 | 88 |
| 3HC | 8000 ± 2000 | 38 |
| ΣHPMA | 742 ± 411 | 75 |
| 2HPhEMA | 7 ± 3 | 25 |
| 122CVMA | 13 ± 5 | 75 |
| BzMA | 18 ± 13 | 75 |
| ΣMHA | 110 ± 25 | 50 |

### XVIII. Comparison of Per Capita Mass Loadings of VOC Metabolites between Communities

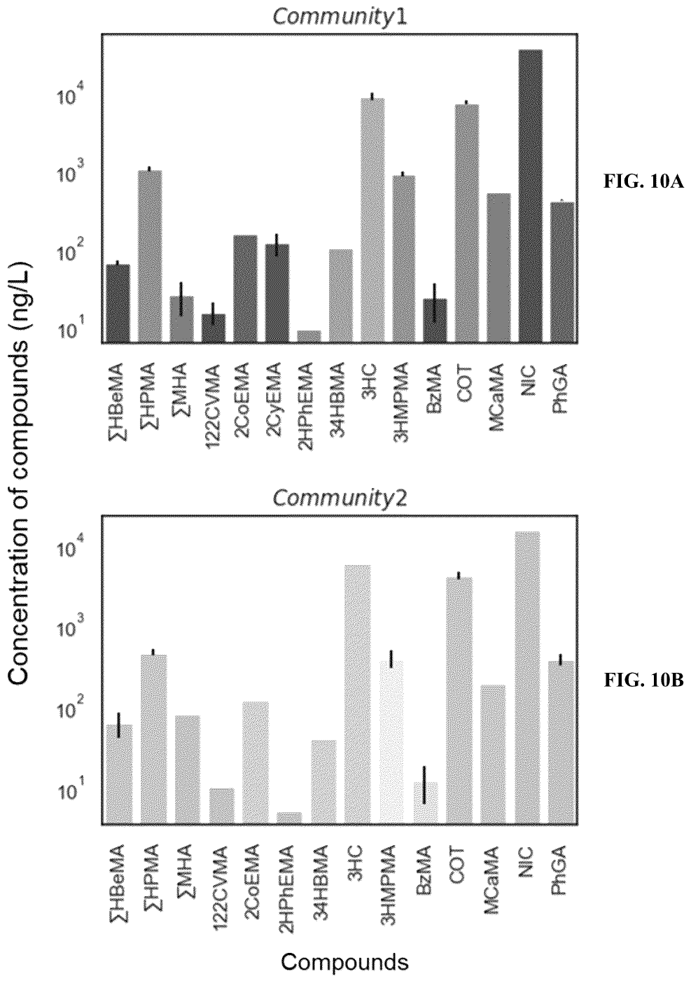

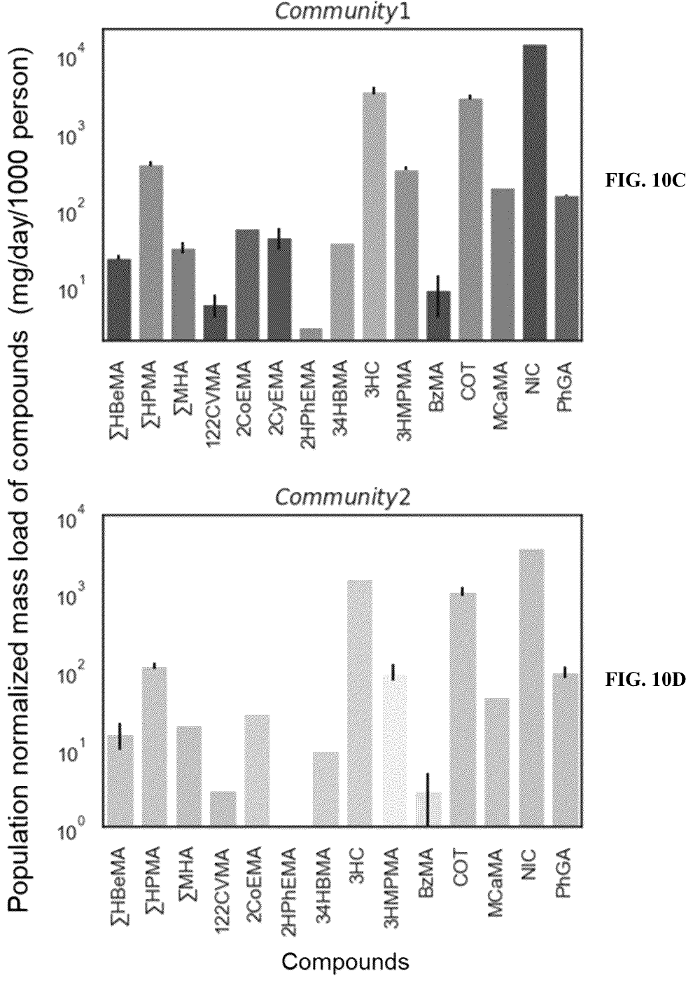

A comparison between the two communities showed that the number of detects across all target analytes within Community 1 (closer proximity to manufacturing facilities) was 36, while Community 2 had 28 detects. Both locations shared the same target compounds except for one additional VOC metabolite 2CyEMA measured only in Community 1, indicating exposure to acrylonitrile. The average measured concentrations, mass loads, and per capita mass loads were systematically higher in Community 1 than in Community 2 (FIGS. 10A-10D), with the total sum of target analyte loading 22,000 mg day⁻¹ per 1000 people in Community 1 versus only 7100 mg day⁻¹ per 1000 people in Community 2 (Community 2 was 25-67% lower). The three nicotine-specific metabolites (NIC, 3HC, COT) represented the highest VOC metabolite loading in the 1000s of mg per day⁻¹ per 1000 people in both locations. Removing these contributions left total loadings at 1750 and 573 mg day⁻¹ per 1000 people, respectively, in the two communities.

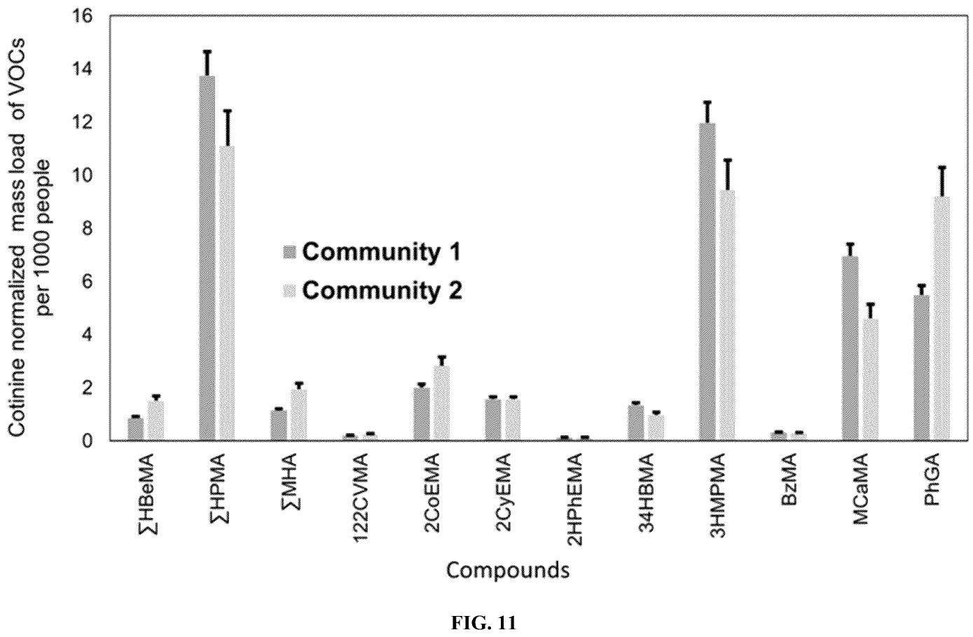

One challenge to interpreting the resultant data are that all biomarkers except for 122CVMA (Tetrachloroethylene), 11DCVMA and 22DCVMA (Trichloroethylene; not detected, qualitative detect), and 1PMA (1-bromopropane; not detected) are found in cigarette smoke, and Community 1 had higher smoking rates as evidenced by higher per capita mass loads of nicotine-specific compounds. To mitigate the differences in exposure related to smoking, we normalized mg day⁻¹ per 1000 people loading of each non-nicotine VOC to cotinine, one of the major acute metabolites of nicotine consumption (Gao et al., 2018). Results showed that with normalization, Community 1 was no longer systematically higher than Community 2 (FIG. 11). Community 2 was, in fact, higher in PhGA (ethylbenzene exposure) and, to a modest extent, 2CoEMA (acrolein), ΣMHA (xylene), and ΣHBeMA (1,3 butadiene). PhGA indicates ethylbenzene exposure, a naturally occurring VOC in crude oil, with exposures related to vehicular traffic and manufacturing. It is also a component in asphalt and consumer products such as paints, varnishes, and pesticides. With other vehicular-related VOC metabolites not measured at elevated levels in this community, the result suggests another source of exposure. 2CoEMA was also higher in Community 2 and is the unique indicator of acrolein exposure. Vehicular exhaust makes up 75% of the total acrolein in outdoor environments; however indoor air concentrations are generally higher with exposures attributed to tobacco smoke and cooking with oils. Again, considering a lack of systematic increases in vehicle-related VOCs, and controlling for smoking levels, this result may be from meal preparation methods.

In general, Community 1, with closer proximity to manufacturing facilities, had higher values across many of the VOC metabolites, even post-normalization to cotinine. The largest differences included ΣHPMA (indicative of propylene oxide and/or acrolein exposure), 3HMPMA (crotonaldehyde), MCaMA (DMF), and 2CyEMA (acrylonitrile), measured in Community 1 only. Propylene oxide, crotonaldehyde, DMF, and acrylonitrile are generally sourced from manufacturing activities and vehicle emissions.

Thus, these preliminary findings indicate urinary biomarkers are detectable in wastewater, and the resultant differences between communities indicate WBE may be used in the future as a tool to assess VOC exposure at the population-level.

What is claimed is:

1. A method of assessing a population's health comprising:
providing a wastewater sample from the population; and
determining the concentration of at least one analyte in the wastewater sample, wherein the at least one analyte is selected from the group consisting of: a hunger hormone, a stress hormone, a cardiovascular disease biomarker, a pulmonary disease biomarker, a cancer biomarker, an illicit drug, a personal care product, a surfactant, a hazardous chemical, a disease resistant pathogen, an antimicrobial resistance gene, a psychotropic drug, an alcohol-related chemical biomarker, a metabolite of volatile organic compounds (VOCs), an environmental toxin, a nicotine-related metabolite, a plastic component, and an endogenous mental health marker.

2. The method of claim 1, wherein the wastewater sample is composited to a neighborhood level and the population is a neighborhood.

3. The method of claim 2, wherein the wastewater sample from the sewage source is composited to a building level and the population is a building's occupants.

4. The method of claim 2, wherein the wastewater sample from the sewage source is composited to a household level and the population is a household.

5. The method of claim 1, further comprising normalizing the concentration of the at least one analyte in the wastewater sample with at least one normalization agent.

6. The method of claim 1, further comprising:
freezing the composited wastewater sample; and
thawing the composited wastewater sample to provide the wastewater sample.

7. The method of claim 5, wherein the concentration of at least one analyte is determined in the thawed fluid sample within two weeks of collecting the fluid sample.

8. The method of claim 1, wherein the plastic component is selected from the group consisting of: bisphenol A (BPA), BPA monosulfate, Bisphenol S, monobenzyl phthalate (MBzP), mono (2-ethyl-5-hydroxyhexyl) phthalate (MEHHP), mono-2-ethylhexyl phthalate (MEHP), monoethyl phthalate (MEP), monobutyl phthalate (MMP), mono-n-octyl phthalate (MnOP), terephthalic acid (TPA), monobutyl phthalate (MBP), monoisobutyl phthalate (MiBP), and mono-(2-ethyl-5-oxohexyl) phthalate (MEOHP).

9. The method of claim 1, wherein:
the cardiovascular disease biomarker is cystatin C; and
the cancer biomarker is microtubule assisted serine/threonine kinase 4 (MAST4);
the method further comprises identifying the population as containing a significant portion of subjects with cardiovascular disease and/or cancer upon detection of the cancer biomarker at a concentration of at least 0.1 ng/L/10,000 population or of the cardiovascular disease biomarker at a concentration of at least 1.2×10³ ng/L/10,000 population.

10. The method of claim 9, wherein the cardiovascular disease biomarker further comprises a biomarker selected from cardiac troponin I (cTnI), α-fetoprotein, and normetanephrine, the method further comprises identifying the population as containing a significant portion of subjects with cardiovascular disease upon detection of cTnI, α-fetoprotein, or normetanephrine at a concentration of at least 0.1 ng/L/10,000 population.

11. The method of claim 1, wherein the metabolite of VOCs is selected from the group consisting of: N-acetyl-S-(2-carboxyethyl)-L-cysteine (2CoEMA), N-acetyl-S-(3-hydroxypropyl)-L-cysteine (3HPMA), N-acetyl-S-(2-cyanoethyl)-L-cysteine (2CyEMA), N-acetyl-S-(3,4-dihydroxybutyl)-L-cysteine (34HBMA), N-acetyl-S-(4-hydroxy-2-buten-1-yl)-L-cysteine ((E)-4HBeMA), N-acetyl-S-(3-hydroxypropyl-1-methyl)-L-cysteine (3HMPMA), N-acetyl-S-(N-methylcarbamoyl)-L-cysteine (MCaMA), phenylglyoxylic acid (PhGA), nicotine (NIC), cotinine (COT), 3-hydroxycotinine (3HC), N-acetyl-S-(2-hydroxypropyl)-L-cysteine (2HPMA), N-acetyl-S-(1-phenyl-2-hydroxyethyl)-L-cysteine (2HPhEMA), N-Acetyl-S-(trichlorovinyl)-L-cysteine (122CVMA), N-acetyl-S-(benzyl)-L-cysteine (BzMA), 2-methylhippuric acid (2MHA), 3-methylhippuric acid (3MHA), and 4-methylhippuric acid (4MHA).

12. The method of claim 1, wherein the analyte concentration determined in the wastewater sample is the metabolite of VOCs, the method further comprises:
separating components of the wastewater sample using liquid chromatography (LC) or gas chromatography (GC);
detecting the separated components of the wastewater sample using a detector;
wherein the detector is selected from the group consisting of: mass spectrometry (MS), flame ionization detection (FID), and electron capture detection (ECD);
spiking the wastewater sample with a mixture of stable isotype-labeled international standards to produce a spiked wastewater sample; and
diluting the spiked wastewater sample prior to detecting the concentration of the metabolite of VOCs.

13. The method of claim 11, further comprising normalizing the concentration of the at least one VOC analyte in the wastewater sample with at least one normalization agent.

14. The method of claim 13, wherein the at least one normalization agent comprises cotinine.

15. The method of claim 13, further comprising normalizing the concentration of the at least one VOC analyte in the wastewater sample for nicotine consumption variability.

16. The method of claim 15, wherein cotinine is used to normalize the concentration of the at least one VOC analyte in the wastewater sample for nicotine consumption variability.

17. The method of claim 1, wherein the concentration of the at least one analyte is determined using at least one method selected from the group consisting of: liquid chromatography (LC), gas chromatography (LC), mass spectrometry (MS), flame ionization detection (FID), and electron capture detection (ECD).

18. The method of claim 17, wherein the concentration of the at least one analyte is determined using LC and one other method.

19. The method of claim 18, wherein the concentration of the at least one analyte is determined using LC-MS.

20. A method of assessing a population's health comprising:
providing a first wastewater sample from the population;
detecting the concentration of at least one analyte in the first wastewater sample, wherein the at least one analyte is selected from the group consisting of: a hunger hormone, a stress hormone, a cardiovascular disease biomarker, a pulmonary disease biomarker, a cancer biomarker, an illicit drug, a personal care product, a surfactant, a hazardous chemical, a disease resistant pathogen, an antimicrobial resistance gene, a psychotropic drug, an alcohol-related chemical biomarker, a metabolite of volatile organic compounds (VOCs), an environmental toxin, a nicotine-related metabolite, a plastic component, and an endogenous mental health marker;
providing a second wastewater sample from the population, wherein the second wastewater sample is collected from a same location as the first wastewater sample and is collected at least two days after collecting the wastewater fluid sample;
detecting the concentration of at least one analyte in the second wastewater sample; and
identifying a change in the population's health upon detection that the concentration of the at least one biomarker is changed between the first wastewater sample and the second wastewater sample.
